(19) United States
(12) Patent Application Publication
(10) Pub. No.: US 2002/0099295 A1
(43) Pub. Date: Jul. 25, 2002

(54) System and method for functional brain mapping and an oxygen saturation difference map algorithm for effecting same

(75) Inventors: Amir Gil, Kiryat Tivon (IL); Tamir Gil, Haim Meuhad (IL); Eli Horn, Kiryat Mozkin (IL); Yuval Garini, Doar Na Misgav (IL)

Correspondence Address: G.E. EHRLICH (1995) LTD., c/o ANTHONY CASTORINA, SUITE 207, 2001 JEFFERSON DAVIS HIGHWAY, ARLINGTON, VA, 22202, US

(73) Assignee: Applied Spectral Imaging Ltd.

(21) Appl. No.: 09/776,871

(22) Filed: Feb. 6, 2001

Related U.S. Application Data
(63) Continuation-in-part of application No. 09/711,521, filed on Nov. 14, 2000.
(60) Provisional application No. 60/167,622, filed on Nov. 26, 1999.

(51) Int. Cl. A61B 5/00

(52) U.S. Cl. 600/476; 600/323; 382/128

(57) ABSTRACT

A method of functional brain mapping of a subject is disclosed. The method is effected by (a) illuminating an exposed cortex of a brain or portion thereof of the subject with incident light; (b) acquiring a reflectance spectrum of each picture element of at least a portion of the exposed cortex of the subject; (c) stimulating the brain of the subject; (d) during or after step (c) acquiring at least one additional reflectance spectrum of each picture element of at least the portion of the exposed cortex of the subject; and (e) generating an image highlighting differences among spectra of the exposed cortex acquired in steps (b) and (d), so as to highlight functional brain regions. Algorithms for calculating oxygen saturation and blood volume maps which can be used to practice the method are also disclosed. Systems for practicing the method are also disclosed.

This is a continuation-in-part of U.S. patent application Ser. No. 09/711,521, filed Nov. 14, 2000, which is a continuation-in-part of U.S. Provisional Patent Application No. 60/167,622, filed Nov. 26, 1999.

### FIELD AND BACKGROUND OF THE INVENTION

The present invention relates to systems and methods for functional brain mapping and further to a novel oxygen saturation and/or blood volume difference map algorithm which can be used for effecting the methods. More particularly, the present invention relates to systems and methods designed for acquiring high spectral and spatial resolution spectral images of an exposed cortex during a neurosurgery, while using peripheral brain stimulation protocols for mapping functional cortical regions and thereby deducing cortical anatomy, especially in cases of distorted anatomy, as is typically the case when a brain space-occupying lesion. e.g., a brain tumor, distorts neighboring brain tissue. Still particularly, the present invention relates to methods and systems for generating and displaying oxygen saturation and/or blood volume difference maps of any tissue, the maps highlighting differences in the oxygen saturation and/or blood volume characterizing the tissue between two time points and typically as a response to stimulation, oxygenation, deoxygenation or blood perfusion.

### Brain Structure-function Relationships

The organization of the different “control centers” in the brain is known as the Homunculus (Latin for “small person”, see FIG. 1) which have been mapped, historically, by examining head injury and stroke cases and the dysfunctions related therewith. In recent years, functional MRI (fMRI), MEG and other techniques were and are still used to improve the homunculus.

These latter studies revealed that the brain does not operate as a simple model where each organ has one well defined “control center”. Brain researchers use the terms “pathways” or “neural pathways”, which constitute axonal connections between different brain functionality centers, to describe the mode of operation of the brain in completing a certain task, such as a motoric task, a vision task, a speech task, etc. Each such task typically involves operation of several distinct function-associated areas of the brain. Evidently, complicated tasks involve more such distinct areas.

The arrangement of these functionality-associated brain areas differs from person to person and is greatly altered or distorted by the presence of a space-occupying lesion (SOL) such as a tumor. Evidently, when a space-occupying lesion is neurosurgically removed, precaution must be taken to avoid damage to neighboring brain tissue, so as to reduce, as much as possible, permanent brain damage to the treated patient.

FIGS. 1a-f show a rough anatomy of the human cortex.

Functionalities associated with the prefrontal area (highlighted in FIG. 2a) include spatial working memory, performance of self-ordered tasks, object and verbal working memory and analytic reasoning.

Functionalities associated with the frontal lobe (highlighted in FIG. 2b) include attention, behavior, abstract thought, reflection, problem solving, creativity, emotion, coordinated movements, generalized and mass movements, some eye movements, muscle movements, intellect, judgment, skilled movements, sense of smell, supplementary motor skills, physical reaction, sexual urges, initiative, inhibition.

Functionalities associated with the parietal lobe (highlighted in FIG. 2d) include appreciation of form through touch (stereognosis), tactile sensation, response to internal stimuli (proprioception), sensory combination and comprehension, some language and reading functions, some visual functions.

Functionalities associated with the occipital lobe (highlighted in FIG. 2e) include reading and vision.

Functionalities associated with the temporal lobe (highlighted in FIG. 2f) include auditory memories, some hearing, visual memories, some vision pathways, other memory functions, music, fear, some language, some speech, some other behavior.

The functionalities associated with the sensorimotor cortex (sensory and motor homunculus) which is highlighted in FIG. 2c are described in more detail in context of FIGS. 3a-b and 4a-b, wherein FIGS. 3a-b show a model of the sensory homunculus and FIGS. 4a-b show a model of the motor homunculus.

Other important brain areas include:

The Wernike's area which forms one of the language regions in the superior temporal gyrus (STG), and which is associated with language comprehension.

The Broca's area, which forms another language region associated with spoken language and language production.

The superior temporal gyros (STG) area (FIG. 5) which includes some or all of the Vemike (which has the ability to “migrate” from its classical location on the STG) and the primary hearing functionality.

The sylvian fissure area, also known as the deep groove, or sulcus, which marks the boundary between the frontal lobe and temporal lobe.

### Brain Tumor Statistics

The Central Brain Tumor Registry of the United States, CBTRUS, supplies the following statistics:

The incidence of primary (both benign and malignant) brain (including all central nervous system tumors) is 11.5 cases per 100,000 person per year. The incidence is higher in males (12.1 per 100,000 person per year) than in females (11.0 per 100,000 person per year). After, Central Brain Tumor Registry of the U.S. data, 1990-1994. U.S. population estimates by age from Census data (03/96).

The incidence of primary benign brain tumors is 4.6 per 100,000 person per year. After, Central Brain Tumor Registry of the U.S. data, 1990-1994. U.S. population estimates by age from Census data (03/96).

In the United States, an estimate of 34,345 new cases of primary benign and malignant brain tumors were expected to be diagnosed in 1998. After, Central Brain Tumor Registry of the U.S. data, 1990-1994. U.S. population estimates by age from Census data (03/96).

The incidence of primary malignant brain tumors is 5.8 cases per 100,000 person per year. This rate is higher in males (7.0 per 100,000 person per year) than in females (4.7 per 100,000 person per year). After, Ries L A G, Kosary C L, Hankey B F, Miller B A, Edwards B K (eds.). SEER Cancer Statistics Review, 1973-1995; National Cancer Institute. Bethesda, Md. 1998. Tables 111(5-7).

In the United States, an estimate of 17,400 new cases of primary malignant brain tumors were expected to be diagnosed in 1998 (9,800 in males and 7,600 in females). After, Ries L A G, Kosary C L, Hankey B F, Miller B A, Edwards B K (eds.). SEER Cancer Statistics Review, 1973-1995; National Cancer Institute. Bethesda, Md. 1998. Tables III(5-7). Table I(1).

The incidence rate of pediatric (ages 0-19 years) primary (benign and malignant) brain tumors is 3.8 cases per 100,000 person per year. The rate is higher in males (4.0 per 100,000 person per year) than in females (3.5 per 100,000 person per year). After, Central Brain Tumor Registry of the U.S. data, 1990-1994. U.S. population estimates by age from Census data (03/96).

An estimate of 2,961 new cases of pediatric primary benign and malignant brain tumors were expected to be diagnosed in 1998. After, Central Brain Tumor Registry of the U.S. data, 1990-1994. U.S. population estimates by age from Census data (03/96).

An estimate of 13,300 deaths were attributed to primary malignant brain tumors in 1998. After, Ries L A G, Kosary C L, Hankey B F, Miller B A, Edwards B K (eds.). SEER Cancer Statistics Review, 1973-1995; National Cancer Institute. Bethesda, MD 1998. Tables 111(5-7). Table I(1).

Males have a 0.65% lifetime risk of being diagnosed with a primary malignant brain tumor and a 0.50% chance of dying from a brain tumor. After, Ries L A G, Kosary C L, Hankey B F, Miller B A, Edwards B K (eds.). SEER Cancer Statistics Review, 1973-1995; National Cancer Institute. Bethesda, Md. 1998. Tables III(5-7). Table III(11).

Females have a 0.52% lifetime risk of being diagnosed with a primary malignant brain tumor and a 0.38% chance of dying from a brain tumor. After, Ries L A G, Kosary C L, Hankey B F, Miller B A, Edwards B K (eds.). SEER Cancer Statistics Review, 1973-1995; National Cancer Institute. Bethesda, Md. 1998. Tables III(5-7). Table III(11).

The five-year relative survival rate following diagnosis of a primary malignant brain tumor (excluding lymphoma) is 26.6% for males and 27.9% for females. Estimated by CBTRUS using SEER Program public use CD-ROM (1973-94), National Cancer Institute, DCPC, Surveillance Program, Cancer Statistics Branch, August 1996.

Five-year relative survival rates following diagnosis of a primary malignant brain tumor by age of diagnosis (data collected from 1973-1994) is: age 0-20 years -59.6%; age 21-44 years -48.1%; age 45-64 years -12.9%; age 65 or older -4.5%. Estimated by CBTRUS using SEER Program public use CD-ROM (1973-94), National Cancer Institute, DCPC, Surveillance Program, Cancer Statistics Branch, August 1996.

### Treatment of Brain Tumors

Given the above grim statistics, neurosurgeons treating brain tumors need to carefully choose the therapeutic option that will result in longer survival periods with minimal damage to life quality. The most common therapeutic options currently available are radiosurgery and chemotherapy which are non invasive, and neurosurgery which is invasive and is, in many cases, performed when the cortex or a portion thereof is exposed, or a combination of the above-mentioned treatments. Each of these approaches has its inherent advantages and disadvantages.

In invasive neurosurgery, the main risk involves causing permanent brain damage to the patient through various complications that might arise, damaging blood vessel's or damaging functional tissue, to name a few, and therefore methods of reducing the of risk of causing permanent brain damage during invasive neurosurgery are in great need.

### Neuronal Activity and Hemodynamic Changes in the Brain

Tight coupling exists between electrical activity in the brain and both cellular metabolism and hemodynamic changes. This tight coupling results from the brain cells lack of capacity of storing energy and has been demonstrated in numerous papers including those by Roy and Sherrington (J. Physiol. 11, 85-108, 1890), Sokolof (J. Neurochem. 28, 897-916, 1977), Chance (Science 137, 499-502, 1962) and others.

The hemodynamic changes include: (i) changes in the level of oxygen saturation (OS) in the local tissue area surrounding activated neurons; and (ii) changes in the cerebral blood volume (CBV) which is caused by changes in cerebral blood flow (CBF).

The metabolic changes include changes in the concentration of diverse compounds such as, but not limited to, glutamate, potassium, cytochrome and other. These changes originate from the chemical reactions that take place when brain neurons are activated.

The hemodynamic coupling is used by modern functional neuroimaging methods such as Positron Emission Tomography (PET), functional MRI (fMRI) and optical imaging to indirectly obtain maps of neuronal activity in the brain.

The above-mentioned techniques are indirect techniques because they measure processes, which are related to the chemical/electrical activity of the brain and not the chemical/electrical activity itself. There exist methods for directly monitoring the electrical activity in the brain, such as EEG and EMG, which will not be further discussed herein, as they are rarely used in intra-operative functional brain mapping.

Thus, all of the brain indirect imaging techniques rely on various types of activity-dependent changes. For example, regional changes in cerebral blood flow (CBF) were first used for functional mapping of the brain by PET studies and subsequently by single photon emission computed tomography (SPECT) and by flow-sensitive MRI.

Optical imaging techniques rely on spectral changes associated with cerebral blood oxygenation changes or blood volume changes (CBV).

During epileptic seizures energetic electrical discharges occur in the brain. These discharges might be local (partial seizure) or general (general seizure). The discharges are followed by hemodynamic changes that can be monitored by MRI imaging. In a paper entitled “Imaging of interictal Epileptiform Discharges Using Spike-triggered fMRI” (IJBEM Number 1, Volume 1, 1999) a group from the Institute of Neurology, UCL, London, UK, showed that the hemodynamic changes involved in focal epilepsy are detectable by fMRI using an EEG based triggering device.

In some cases epilepsy patients undergo neurosurgery. The best candidates for neurosurgery are patients with focal epilepsy. Tests are performed to determine the origin of the seizures within the brain—the seizure focus. If the seizure focus is identified in a discrete, removable part of the brain, resective surgery can be effective. However, some foci are not well localized and others are located in brain areas that cannot be removed.

### Current Functional Brain Mapping Techniques and their Drawbacks

In recent years, the field of neurosurgery has seen many developments intended to help the neurosurgeon in mapping the brain, prior to and during the operation, so as to minimize the brain damage to treated patients.

One of the developments introduced in recent years is known as a brain navigation system. In a brain navigation system, the data collected by a pre-operational, non-invasive, technique (e.g., fMRI, CT, PET) is combined with an accurate three-dimensional (3D) orientation system, thus providing the neurosurgeon with a mechanism with which the neurosurgeon can navigate a surgical tool within the brain while knowing its position, in real-time, relative to pre-operational mapped areas of the brain and relative to, for example, a brain tumor.

This approach, although in some cases very useful, has few drawbacks. First, the instrumentation involved is very expensive and complex. Second, the process of obtaining, for example, fMRI images is time consuming. Last but not least, whenever craniotomy is performed and due to the inner cortical pressure, brain areas change their positions (shift), effecting the accuracy of any pre-craniotomy image.

Although greatly improved, MRI systems are very difficult and inconvenient for use during operation. At present, there are no fMRI systems available for intra-operative use. An fMRI system requires a field of more then 1.5 Tesla where the current intra-operative MRI's are low-field systems characterized by fields of up to 0.5 Tesla.

The exposed cortex, as the neurosurgeon sees it, differs greatly from the computer generated fMRI, or PET, images (see Example 1 of the Examples section below).

In many cases, the neurosurgeon still relies on direct cortical stimulation (e.g., via an electrodes) for registering the exposed cortex with prior knowledge, be it the knowledge of the homunculus or information provided by preoperational imaging results.

### Optical Imaging

The field of optical imaging, including spectral imaging, can be divided into two major categories according to the wavelengths used: (i) optical imaging in the visual range; and (ii) optical imaging in the infrared range, typically the near infrared (NIR) range.

A major difference between the visual range and the NIR range is in the depth of penetration, or, in other words, the depth from which information is obtainable. In a paper from 1983, Svaanand and Ellingsen (Photochem. Photobiol. 38 293-299) measured the optical penetration through human brain tissue and showed that light intensity falls by [symbol] (approximately 37%) every 0.4 mm at 514 nm and every 3.2 mm at 1060 nm.

Several additional considerations and parameters create a difference between working in the visual range as compared to the NIR range, as follows:

First, scattering is a function of the path length in the tissue. Increasing the path length (as in NIR) results in greater scattering, thus complicating spectral calculations.

Second, the optical absorption of hemoglobin (both oxy-hemoglobin and deoxy-hemoglobin) drops drastically at wavelengths above 600 nm. This means that measuring oxy-hemoglobin—deoxy-hemoglobin ratios in the NIR range will be far noisier as is compared to these ratios measured in the visual range.

Third, the optical absorption of cytochrome aa₃ (which is, however, not related to neuronal activity) becomes considerable, amounting for about 10% of the total absorption in the NIR (S. Wray, M. Cope, D. T. Delpy, J. S. Wyatt, and 0. R. Reynolds, Biochimia et Biophysica Acta, 933 (1988)184-192).

Fourth, the spatial resolution in the visible range is superior over that in the NIR range since the spatial resolution is a function of wavelength.

Fifth, presently available infrared devices (detectors, lenses, etc.) are more complicated (and costly) as is compared to similar devices operative in the visual range. Spectral imaging devices in the NIR range are presently not commercially available.

### General Overview of Spectral Imaging

A spectrometer is an apparatus designed to accept light, to separate (disperse) it into its component wavelengths, and measure the lights spectrum, that is the intensity of the light as a function of its wavelength. A spectral imaging device, also referred to herein as “imaging spectrometer” is a spectrometer which collects incident light from a scene and measures the spectra of each picture element thereof.

Spectroscopy is a well known analytical tool which has been used for decades in science and industry to characterize materials and processes based on the spectral signatures of chemical constituents therein. The physical basis of spectroscopy is the interaction of light with matter. Traditionally, spectroscopy is the measurement of the light intensity emitted, scattered or reflected from or transmitted through a sample, as a function of wavelength, at high spectral resolution, but without any spatial information.

Spectral imaging, on the other hand, is a combination of high resolution spectroscopy and high resolution imaging (i.e., spatial information). Most of the works so far described in spectral imaging concern either obtaining high spatial resolution information from a biological sample, yet providing only limited spectral information, for example, when high spatial resolution imaging is performed with one or several discrete band-pass filters [See, Andersson-Engels et al. (1990) Proceedings of SPIE—Bioimaging and Two-Dimensional Spectroscopy, 1205, pp. 179-189], or alternatively, obtaining high spectral resolution (e.g., a full spectrum), yet limited in spatial resolution to a small number of points of the sample or averaged over the whole sample [See for example, U.S. Pat. No. 4,930,516, to Alfano et al.].

Conceptually, a spectral imaging system comprises (i) a measurement system, and (ii) an analysis software. The measurement system includes all of the optics, electronics and the manner in which the sample is illuminated (e.g., light source selection), the mode of measurement (e.g., fluorescence or transmission), as well as the calibration best suited for extracting the desired results from the measurement. The analysis software includes all of the software and mathematical algorithms necessary to analyze and display important results in a meaningful way.

Spectral imaging has been used for decades in the area of remote sensing to provide important insights in the study of Earth and other planets by identifying characteristic spectral absorption features originating therefrom. However, the high cost, size and configuration of remote sensing spectral imaging systems (e.g., Landsat, AVIRIS) has limited their use to air and satellite-born applications [See, Maymon and Neeck (1988) Proceedings of SPIE—Recent Advances in Sensors, Radiometry and Data Processing for Remote Sensing, 924, pp. 10-22; Dozier (1988) Proceedings of SPIE—Recent Advances in Sensors, Radiometry and Data Processing for Remote Sensing, 924, pp. 23-30].

There are three basic types of spectral dispersion methods that might be considered for a spectral bio-imaging system: (i) spectral grating or prism, (ii) spectral filters and (iii) interferometric spectroscopy. As will be described below, the latter is best suited to implement the method of the present invention, yet certain filter-based configurations may also prove applicable.

In a grating or prism (i.e., monochromator) based systems, also known as slit-type imaging spectrometers, such as for example the DILOR system: [see, Valisa et al. (Sep. 1995) presentation at the SPIE Conference European Medical Optics Week, BiOS Europe 1995, Barcelona, Spain], only one axis of a CCD (charge coupled device) array detector (the spatial axis) provides real imagery data, while a second (spectral) axis is used for sampling the intensity of the light which is dispersed by the grating or prism as function of wavelength. The system also has a slit in a first focal plane, limiting the field of view at any given time to a line of picture elements. Therefore, a full image can only be obtained after scanning the grating (or prism) or the incoming beam in a direction parallel to the spectral axis of the CCD in a method known in the literature as line scanning. The inability to visualize the two-dimensional image before the whole measurement is completed, makes it impossible to choose, prior to making the measurement, a desired region of interest from within the field of view and/or to optimize the system focus, exposure time, etc. Grating and prism based spectral imaging devices are in use for remote sensing applications, because an airplane (or satellite) flying over the surface of the Earth provides the system with a natural line scanning mechanism.

It should be further noted that slit-type imaging spectrometers have a major disadvantage since most of the picture elements of one frame are not measured at any given time, even though the fore-optics of the instrument actually collects incident light from all of them simultaneously. The result is that either a relatively large measurement time is required to obtain the necessary information with a given signal-to-noise ratio, or the signal-to-noise ratio (sensitivity) is substantially reduced for a given measurement time. Furthermore, slit-type spectral imaging devices require line scanning to collect the necessary information for the whole scene, which may introduce inaccuracies to the results thus obtained.

Filters-based spectral dispersion methods can be further categorized into discrete filters and tunable filters. In these types of imaging spectrometers the spectral image is built by filtering the radiation for all the picture elements of the scene simultaneously at a different wavelength at a time by inserting, in succession, narrow band pass filters in the optical path, or by electronically scanning the bands using acousto-optic tunable filters (AOTF) or liquid-crystal tunable filter (LCTF), see below. Similarly to the slit type imaging spectrometers equipped with a grating or prism as described above, while using filters-based spectral dispersion methods, most of the radiation is rejected at any given time. In fact, the measurement of the whole image at a specific wavelength is possible because all the photons outside the instantaneous wavelength being measured are rejected and do not reach the CCD.

Tunable filters, such as AOTFs and LCTFs have no moving parts and can be tuned to any particular wavelength in the spectral range of the device in which they are implemented. One advantage of using tunable filters as a dispersion method for spectral imaging is their random wavelength access; i.e., the ability to measure the intensity of an image at a number of wavelengths, in any desired sequence without the use of filter wheels. However, AOTFs and LCTFs have the disadvantages of (i) limited spectral range (typically, λₘₐₓ=2λₘᵢₙ) while all other radiation that falls outside of this spectral range must be blocked, (ii) temperature sensitivity, (iii) poor transmission, (iv) polarization sensitivity, and (v) in the case of AOTFs an effect of shifting the image during wavelength scanning, demanding careful and complicated registration procedures thereafter.

All these types of filter and tunable filters-based systems have not been used successfully and extensively over the years in spectral imaging for any application, because of their limitations in spectral resolution, low sensitivity, and lack of easy-to-use and sophisticated software algorithms for interpretation and display of the data.

A method and apparatus for spectral analysis of images which have advantages in the above respects is disclosed in U.S. Pat. No. 5,539,517, which is incorporated by reference as if fully set forth herein, with the objective to provide a method and apparatus for spectral analysis of images which better utilizes all the information available from the collected incident light of the image to substantially decrease the required frame time and/or to substantially increase the signal-to-noise ratio, as compared to the conventional slit- or filter type imaging spectrometer, and does not involve line scanning. According to this invention, there is provided a method of analyzing an optical image of a scene to determine the spectral intensity of each picture element (i.e., region in the field of view which corresponds to a pixel in an image presenting same) thereof by collecting incident light from the scene; passing the light through an interferometer which outputs modulated light corresponding to a predetermined set of linear combinations of the spectral intensity of the light emitted from each picture element; focusing the light outputted from the interferometer on a detector array, scanning the optical path difference (OPD) generated in the interferometer for all picture elements independently and simultaneously and processing the outputs of the detector array (the interferograms of all picture elements separately) to determine the spectral intensity of each picture element thereof.

This method may be practiced by utilizing various types of interferometers wherein the optical path difference (OPD) is varied to build the interferograms by moving the entire interferometer, an element within the interferometer, or the angle of incidence of the incoming radiation. In all of these cases, when the scanner completes one scan of the interferometer, the interferograms for all picture elements of the scene are completed.

Apparatuses in accordance with the above features differ from the conventional slit- and filter type imaging spectrometers by utilizing an interferometer as described above, therefore not limiting the collected energy with an aperture or slit or limiting the incoming wavelength with narrow band interference or tunable filters, thereby substantially increasing the total throughput of the system. Thus, interferometer-based apparatuses better utilize all the information available from the incident light of the scene to be analyzed, thereby substantially decreasing the measurement time and/or substantially increasing the signal-to-noise ratio (i.e., sensitivity). The sensitivity advantage that interferometric spectroscopy has over the filter and grating or prism methods is known in the art as the multiplex or Fellgett advantage [see, Chamberlain (1979) The principles of interferometric spectroscopy, John Wiley and Sons, pp. 16-18 and p. 263].

In U.S. Pat. No. 5,748,162, which is incorporated by reference as if fully set forth herein, the objective was to provide spectral imaging methods for biological research, medical diagnostics and therapy, which methods can be used to detect spatial organization (i.e., distribution) and to quantify cellular and tissue natural constituents, structures, organelles and administered components such as tagging probes (e.g., fluorescent probes) and drugs using light transmission, reflection, scattering and fluorescence emission strategies, with high spatial and spectral resolutions.

Other uses of the spectral imaging device described in U.S. Pat. No. 5,539,517 are described in the U.S. Pat. No. 6,088,099 “Method for interferometer based spectral imaging of moving objects”, U.S. Pat. No. 6,075,599 “Optical device with entrance and exit paths that are stationary under device rotation”; U.S. Pat. No. 6,066,459 “Method for simultaneous detection of multiple fluorophores for in situ hybridization and multicolor chromosome painting and banding”; U.S. Pat. No. 6,055,325 “Color display of chromosomes or portions of chromosomes” U.S. Pat. No. 5,043,039 “Method of and composite for in situ fluorescent hybridization” U.S. Pat. No. 6,018,587 “Method for remote sensing analysis be decorrelation statistical analysis and hardware therefor”; U.S. Pat. No. 6,007,996 “In situ method of analyzing cells”; U.S. Pat. No. 5,995,645 “Method of cancer cell detection”; U.S. Pat. No. 5,991,028 Spectral bio-imaging methods for cell classification”; U.S. Pat. No. 5,936,731 “Method for simultaneous detection of multiple fluorophores for in situ hybridization and chromosome painting”; U.S. Pat. No. 5,912,165 “Method for chromosome classification by decorrelaiton statistical analysis and hardware therefore”; U.S. Pat. No. 5,906,919 “Method for chromosomes classification”; U.S. Pat. No. 5,871,932 “Method of and composite for fluorescent in situ hybridization”; U.S. Pat. No. 5,856,871“Film thickness mapping using interferometric spectral imaging”; U.S. Pat. No. 5,835,214 “Method and apparatus for spectral analysis of images”; U.S. Pat. No. 5,834,203 “Method for classification of pixels into groups according to their spectra using a plurality of wide band filters and hardware therefore”; U.S. Pat. No. 5,817,462 “Method for simultaneous detection of multiple fluorophores for in situ hybridization and multicolor chromosome painting and banding”; U.S. Pat. No. 5,798,262 “Method for chromosomes classification”; U.S. Pat. No. 5,784,162 “Spectral bio-imaging methods for biological research, medical diagnostics and therapy”; U.S. Pat. No. 5,719,024 “Method for chromosome classification by decorrelation statistical analysis and hardware therefore, all of which are incorporated herein by reference.

Spectral bio-imaging systems are potentially useful in all applications in which subtle spectral differences exist between chemical constituents whose spatial distribution and organization within an image are of interest. The measurement can be carried out using virtually any optical system attached to the system described, for example, in U.S. Pat. No. 5,539,517, for example, an upright or inverted microscope, a fluorescence microscope, a macro lens, an endoscope and a fundus camera. Furthermore, any standard experimental method can be used, including light transmission (bright field and dark field), auto-fluorescence and fluorescence of administered probes, etc.

### Attempts Made at Detecting Brain Hemodynamics by Use of Optical and Spectral Imaging Methods

The use of spectral and optical imaging devices for neurosurgery has been demonstrated in several papers and has also been the subject matter of several patents, as is further discussed below.

In a paper from 1992 (Nature, 358: 668-671) Haglund, Ojemann and Hochman reported functional brain mapping using an optical imaging system to obtain maps of the human cortex during stimulation-evoked epileptiform after discharges and cognitively evoked functional activity. They show that optical changes increased in magnitude as the intensity and duration of the after discharges increased and show their ability to detect optical changes during speech and performance of cognitive tasks. This paper introduces optical imaging as a potential technique for use in the neurosurgical operating room.

In a paper from 1996 (Science; 272:551-554) Grinvald and Malonek explain the interactions between electrical activity and cortical microcirculation as revealed by imaging spectroscopy. In this work, a slit grating spectral imaging device was used to study the dynamic changes in microcirculation, mainly the dynamic changes in oxy-hemoglobin and deoxy-hemoglobin concentration, in response to stimulation. This paper also addresses the relations between area sizes that are associated with the first, highly localized, tissue hypoxia and the latter (more then 3 seconds post stimulation), less local, vascular response. In a later work (PNAS, 94:1486-14831, 1997), Grinvald continued the research in the field of brain hemodynamics using another grating spectral imaging device (a Laser Doppler flowmeter).

As is further detailed hereinabove, the use of grating spectral imaging devices has a major drawback for brain mapping as it is limited to collecting data from one column (or row) at a time in what is known as raster scanning. However, due to the tight coupling of neural activity in the brain with hemodynamic changes and/or cellular functionality, it fails to provide a comprehensive functionality map of the brain since data from each column (or row) is collected at a different time.

U.S. Pat. No. 5,215,095 entitled “Optical imaging system for neurosurgery” teaches another approach. According to this patent a CCD device is connected to a surgical microscope, imaging the cortex by using the optics and illumination of the surgical microscope and using one or none filters for filtering the reflected light. Sets of reflectance intensity images of the brain are acquired, in real time. The signal-to-noise ratios of these intensity images are improved by averaging on 128 frames per each image and changes between different images taken in sequence are extracted therefrom by subtracting the base images (performed first) from the images collected during sensory or other stimulation (the exact calculation and its underlying physiology are not given). An additional set of images is collected, in the same manner after neuronal activation for analyzing the recovery process.

This patent relates to measuring one of two physiological processes: (i) using a 800 nm band-pass filter the system is reported to detect functional activity by recording changes related to movement of ions and water from the extracellular space into neurons, swelling of the cells, shrinkage of the extracellular space and neurotransmitter release; and (ii) using a filter in the 500-700 nm or no filter (white light), the system is reported to detects functional activity by recording changes related to hemodynamics.

U.S. Pat. No. 5,198,977, entitled “System and method for localization of functional activity in the human brain” describes the use of a flash lamp illuminating the cortex through a two-position filter-wheel where the reflected signal is then recorder by a video camera. The filter layers so recorded are then used for calculating and presenting gray-scale or color-scale maps representing total hemoglobin concentration on the cortex, at any given point in time, and maps representing the difference in hemoglobin concentration before and after functional activation of the brain. No particulars are disclosed with respect to any of these calculations.

When imaging an exposed human cortex, during neurosurgical procedure, one encounters a few problems:

First, the exposed brain area is usually large, typically in the range of 10×10 cm. Achieving homogeneous illumination over such a large area is not a simple task. Furthermore, the exposed cortex is, in general, a non-smooth, curved surface, even more complicating the illumination task.

Second, the brain beats in a beating rate which is correlated to the heart beating rate of the patient. The beat induced spatial modulation of an exposed cortex is significant, of the magnitude of 1 cm for a large craniotomy. This brain beating constantly changes the reflectance intensity from the cortex and does so in a manner that is different for different areas of the cortex.

Third, the time scale of a hemodynamic processes is in the order of one second. Achieving images with good signal-to-noise ratio at a rate of more then 2 per second (which is what one would need in order to detect changes in the interval of 1 second) is a very difficult task. In fact, presently it is an impossible task. Indeed, the spectral data collected from brains as described hereinabove is of low signal-to-noise ratio, and of poor spatial and/or spectral resolutions.

Fourth, in some cases cortical active regions might stay active throughout the entire operation, rendering such regions indistinguishable by a differences based system. In awake patients, who are the preferred population for functional brain mapping, as such patients can be asked to perform different tasks during operation, the somatosensory cortex and the speech center are both constantly activated due to the fact that such patients have lines connected into their arms and legs and that such patients oral responses are frequently requested by the operating staff throughout the operation. Under such circumstances, any attempt to map the somatosensory cortex and/or the speech center with a difference based system should fail.

Soenksen and Garini have demonstrated the use of a Fourier transform (interferometer-based) spectral-imaging device as an oxymeter, of the exposed cortex, in a paper from 1996 (Proceedings of SPIE, 2679:182-189). In this work, spectral images of an exposed rat cortex have been acquired while changing the respiration condition of the experimental animal. The work followed the spectral changes observed between images taken in different respiration conditions (normoxia and anoxia) and between different anatomical areas of the cortex (vein, artery and brain tissue). The paper states that the acquired reflectance spectra can provide the basis for constructing oxygen saturation (OS) maps of the cortex, however it fails to teach how to do so.

In a paper from September 1997 (Biophysical Journal, 73:1223-1231) a team from Carnegie Mellon University has presented their results of oxygen saturation and tension mapping of mice cortex with an acousto-optic tunable filter (AOTF) spectral imaging device. In this work the oxygen saturation of the mice cortex was measured while changing the inspiratory O₂ level from hypoxia (10% O₂) through normoxia (21% O₂) to hyperoxia (60% O₂), by acquiring images at different wavelengths and calculating the oxygen saturation map. The above work is a true spectral-imaging approach to oxygen saturation mapping. Nevertheless, the results presented in this work, ultimately, prevent it from becoming an application suitable for the operating room. The major contributors to this shortcoming include the following:

The AOTF inherently suffers from a low throughput. This is seen when studying the data presented in the work:

The total acquisition time used for reconstituting a single spectral-image was 75 seconds. This is far too long an interval for detecting hemodynamic processes that reflect motor tasks, such as a hand moving, mouth open/close tasks, etc. as task performance under operation room conditions should be short as possible, typically limited to 10-20 seconds.

Because of the low throughput, a powerful light source was used (75 W Xenon arc lamp with a flux of 1000 lm), to illuminate a sample area of less than 1×1 mm. The energy required for illuminating a typical human craniotomy (about 10×10 cm), while maintaining the same flux, would be 10,000 times greater. A light source with a luminous flux of 10 million lm cannot be used within an operating room.

Six×six binning was used to improve signal to noise ratio. This will affect the quality of the spectral resolution offered by this kind of device and implies of the low throughput.

The algorithm used for calculation of the oxygen saturation was based on a least-squares method, while the mathematical model describing the absorption behavior is a linear combination. Furthermore, in the paper it is mentioned that the tissue area that was taken as a reference was assumed to contain no hemoglobin. This assumption is not valid as the magnification used in the experiment (10×) does not allow for discriminating the smallest blood vessels within the tissue, and is the reason why the oxygen saturation maps (e.g., FIG. 4 therein) fail to map the oxygen saturation level of the tissue, thus making anatomy mapping impossible.

As a consequence of the above, the results quoted in the paper are of the following accuracy: At 10% O₂ respiration, oxygen saturation is 61±12% for an artery and 22±10% for a vein. For the other two respiratory values (21% and 60%) no error numbers are given. This accuracy is too low for any useful quantitative oxygen saturation mapping of the cortex.

There is thus a widely recognized need for, and it would be highly advantageous to have, a system and method useful for functional brain mapping, devoid of the above limitations.

### SUMMARY OF THE INVENTION

According to one aspect of the present invention there is provided a method of functional brain mapping of a subject comprising the steps of (a) illuminating an exposed cortex of a brain or portion thereof of the subject with incident light, typically regulated (filtered) broad spectrum light; (b) acquiring a reflectance spectrum of each picture element of at least a portion of the exposed cortex of the subject; (c) stimulating the brain of the subject (e.g., inducing brain activity); (d) during or after step (c) acquiring at least one additional reflectance spectrum of each picture s element of at least the portion of the exposed cortex of the subject; and (e) generating an image highlighting differences among spectra of the exposed cortex acquired in steps (b) and (d), so as to highlight functional brain regions.

According to another aspect of the present invention there is provided a method of performing a neurosurgery for the removal of a mass from a brain of a subject while minimizing damage to a neighboring brain tissue, the method comprising the steps of (a) performing a craniotomy so as to expose at least a portion of a cortex of the subject; (b) performing functional brain mapping of the subject by (i) illuminating the exposed portion of the cortex with incident light; (ii) acquiring a reflectance spectrum of each picture element of at least a portion of the exposed cortex of the subject; (iii) stimulating the neighboring brain tissue of the subject (e.g., inducing brain activity); (iv) during or after step (iii) acquiring at least one additional reflectance spectrum of each picture element of at least the portion of the exposed cortex of the subject; and (v) generating an image highlighting differences among spectra of the exposed cortex acquired in steps (ii) and (iv), so as to highlight the functional brain regions of the neighboring brain tissue; and (c) assisted by the image, removing the mass while minimizing damage to the neighboring brain tissue.

In one particular embodiment the method comprising the steps of (a) performing a craniotomy so as to expose at least a portion of a cortex of the subject; (b) performing functional brain mapping of the subject by (i) illuminating the exposed portion of the cortex with regulated broad spectrum light; (ii) acquiring the reflectance spectrum of each picture element of at least a portion of the exposed cortex of the subject and calculating the oxygen saturation within the exposed cortex area; (iii) evoking neural stimulation, to the patient's brain, in a manner that induces neuronal activity in some or all of the exposed portion of the cortex; (iv) during or after step (iii) acquiring at least one additional reflectance spectrum of each picture element of at least the portion of the exposed cortex of the subject and calculating the oxygen saturation within the exposed cortex area; and (v) generating an image highlighting differences of oxygen saturation on the exposed cortex acquired in steps (ii) and (iv), so as to highlight the functional brain regions in the exposed brain tissue; and (c) assisted by the image, plan the neurosurgical procedure in a manner that will help in minimizing dysfunction to the patient.

According to yet another aspect of the present invention there is provided a system for functional brain mapping of a subject, the system comprising (a) an illumination device for illuminating an exposed cortex of a brain or portion thereof of the subject with incident light; (b) a spectral imaging device for acquiring reflectance spectra of each picture element of at least a portion of the exposed cortex of the subject before and during and/or after stimulating the brain of the subject; and (c) an image generating device for generating an image highlighting differences among spectra of the exposed cortex acquired before and during and/or after stimulating the brain of the subject, so as to highlight functional brain regions.

In a preferred embodiment of the invention, a plurality of images highlighting differences among spectra are displayed either superimposed, overlaid or integrated, so as to provide a cumulative differences display or map of the spectral changes in the tissue.

According to further features in preferred embodiments of the invention described below, the method further comprising the step of using at least one filter to adjust the spectrum of the incident light. Accordingly, the system further comprising at least one filter engaged with the illumination device to adjust the spectrum of the incident light.

According to still further features in the described preferred embodiments each of steps (b) and (d) of the first method (or steps (ii) and (iv) of the second method) is independently characterized by spectral resolution ranging between 1 nm and 50 nm and spatial resolution ranging between 0.1 mm and 1.0.

Accordingly, the system is so designed and constructed so as to provide spectral resolution ranging between 1 nm and 50 nm and spatial resolution ranging between 0.1 mm and 1.0 mm.

According to still further features in the described preferred embodiments each of steps (b) and (d) of the first method (or steps (ii) and (iv) of the second method) is effected via an interferometer-based spectral imaging device.

According to still further features in the described preferred embodiments each of steps (b) and (d) of the first method (or steps (ii) and (iv) of the second method) is effected via a filters-based spectral imaging device. In this case, illumination is preferably effected by an illumination device operated with an alternating current characterized by a frequency time wherein (i) an exposure time of all filters of the filters-based spectral imaging device is substantially equal; and (ii) an exposure time of each of the filters is a multiplicity of the frequency time by an integer.

According to still further features in the described preferred embodiments the method further comprising the steps of generating individual spectra-images from spectra acquired in steps (b) and (d) of the first method (or steps (ii) and (iv) of the second method). Thus, the image generating device is designed and constructed for generating individual spectra-images from spectra of the exposed cortex acquired before and during and/or after stimulating the brain of the subject.

According to still further features in the described preferred embodiments the spectral-images are generated by attributing each of the pixels in the images a distinctive color or intensity according to oxygen saturation and/or blood volume characterizing its respective picture element in the cortex.

According to still further features in the described preferred embodiments the subject is awake.

According to still further features in the described preferred embodiments the subject is anesthetized.

According to still further features in the described preferred embodiments step (c) is effected by asking the subject to perform a task.

According to still further features in the described preferred embodiments the task is selected from the group consisting of reading, speaking, listening, viewing, memorizing, thinking and executing a voluntary action.

According to still further features in the described preferred embodiments step (c) of the first method (or step (iii) of the second method) is effected by passively stimulating the brain of the subject.

According to still further features in the described preferred embodiments the method further comprising the step of generating an anatomical image of the exposed cortex and co-displaying the image highlighting differences among spectra of the exposed cortex and the anatomical image of the exposed cortex. Thus, the image generating device is designed and constructed for generating an anatomical image of the exposed cortex and co-displaying the image highlighting differences among spectra of the exposed cortex and the anatomical image of the exposed cortex.

According to still further features in the described preferred embodiments the image highlighting differences among spectra of the exposed cortex and the anatomical image of the exposed cortex are co-displayed side by side.

According to still further features in the described preferred embodiments the image highlighting differences among spectra of the exposed cortex and the anatomical image of the exposed cortex are superimposed.

According to still further features in the described preferred embodiments the anatomical image includes text identifying brain portions.

According to still further features in the described preferred embodiments elements containing orientation related symbols, such as text, are placed on the exposed portion of the cortex prior to imaging so as to provide an image in which orientation is inherent.

According to still further features in the described preferred embodiments the orientation elements are also used as a “white target” and are later used by the analysis software for providing a “white target” spectrum for further calculations. The white target is preferably selected having an index of refraction close to (say ±10% or ±20%) the index of refraction of a cortex, say of a human cortex.

According to still further features in the described preferred embodiments step (e) comprises a use of at least one threshold while generating the image highlighting differences among spectra of the exposed cortex acquired in steps (b) and (d) of the first method (or steps (ii) and (iv) of the second method). Thus, the image generating device uses at least one threshold while generating the image highlighting differences among spectra of the exposed cortex.

According to still further features in the described preferred embodiments the image highlighting differences among spectra of the exposed cortex is color or intensity coded.

Thus, according to still further features in the described preferred embodiments the image highlighting differences is color-coded according to the set thresholds in such a way that, for example, image pixels fulfilling the condition set by the threshold are colored and all pixels not fulfilling the condition set by the threshold are not colored. Pixels colored in different colors representing fulfillment of more then one threshold can also be used.

According to still further features in the described preferred embodiments medical lines are connected to the subject on a single side thereof.

According to still further features in the described preferred embodiments medical lines are connected to the subject at locations which are less communicating with the exposed portion of the cortex of the subject.

According to still further features in the described preferred embodiments step (e) of the first method (or step (v) of the second method) is characterized by highlighting oxygen saturation and/or blood volume differences of about at least 5% or at least 10%. Thus, the image generating device is set to highlight oxygen saturation and/or blood volume differences of about at least 5% or at least 10%.

As used herein in the specification and the claims section that follows, the term about refers to ±20%.

According to still further features in the described preferred embodiments the method further comprising the step of also acquiring a reflectance spectrum of each picture element of at least the portion of the exposed cortex of the subject when the patient is briefly anesthetized.

According to still further features in the described preferred embodiments each of steps (b) and (d) of the first method (or steps (ii) and (iv) of the second method) is performed during at least N brain beats of the subject, wherein N is an integer selected from the group consisting of two, three, four, five, six, seven, eight, nine, ten and an integer between and including eleven and forty.

According to still further features in the described preferred embodiments step (d) of the first method (or step (iv) of the second method) is executed more than about 3-5 seconds and preferably between about 5 and about 30 seconds after the initiation of step (c) of the first method (or step (iii) of the second method).

According to still further features in the described preferred embodiments the stimulation prolongs about 5 to about 30 seconds, preferably about 10 to about 20 seconds.

According to still further features in the described preferred embodiments the filters-based spectral imaging device includes filters selected so as to collect spectral data of intensity peaks or steeps characterizing one or more spectrally monitored substances.

According to still further features in the described preferred embodiments the filters-based spectral imaging device includes filters selected so as to collect spectral data of intensity peaks or steeps characterizing hemoglobin selected from the group consisting of deoxy-hemoglobin, oxy-hemoglobin and deoxy-hemoglobin and oxy-hemoglobin.

According to still further features in the described preferred embodiments each of the filters is individually about 5 to about 15 nm full-width-at-half-maximum filter.

According to still further features in the described preferred embodiments each of the filters is individually about 10 nm full-width-at-half-max filter.

According to still further features in the described preferred embodiments the filters include N filters selected from the group consisting of an about 540 nm maximal transmittance filter, an about 575 nm maximal transmittance filter, an about 555 nm maximal transmittance filter, an about 513 nm maximal transmittance filter and an about 600 nm maximal transmittance filter, whereas N is an integer selected from the group consisting two, three, four and five.

According to still further features in the described preferred embodiments the filters include at least one multiple chroic filter, such as dichroic filter or trichroic filter. Such a filter can replace a pair or triad of monochroic filters.

According to still further features in the described preferred embodiments the filters include at least one filter of maximal transmittance at a wavelength which corresponds to at least one isosbasthic point of deoxy-hemoglobin and oxy-hemoglobin and at least one additional filter of maximal transmittance at a wavelength which corresponds to at least one non-isosbasthic point of deoxy-hemoglobin and oxy-hemoglobin.

According to still further features in the described preferred embodiments the reflectance spectrum of steps (b) or (d) of the first method (or (ii) or (iv) of the second method) is an averaged reference spectrum of N measurements or brain beats, wherein N is an integer and equals at least 2 and is preferably between 5 and 20, say about 10.

According to still further features in the described preferred embodiments the method further comprising the step of spatially registrating spectral data. Thus, the spectral imaging device is designed and constructed for spatially registrating spectral data acquired thereby.

According to still further features in the described preferred embodiments the method further comprising the step of normalizing spectral data. Thus, the spectral imaging device is designed and constructed for normalizing spectral data acquired thereby, e.g., via a suitable normalizing algorithm.

According to still further features in the described preferred embodiments the image highlighting differences among spectra of the exposed cortex is highlighting oxygen saturation and/or blood volume differences.

According to still further features in the described preferred embodiments at least one threshold is used while generating the image highlighting differences among spectra of the exposed cortex of oxygen saturation and/or blood volume differences.

According to still further features in the described preferred embodiments the at least one threshold includes taking into account only picture elements in which an absolute oxygen saturation and/or blood volume is above a predetermined first threshold.

According to still further features in the described preferred embodiments the at least one threshold further includes taking into account only picture elements in which a difference in oxygen saturation and/or blood volume is above a predetermined second threshold.

According to still further features in the described preferred embodiments clusters of neighboring picture elements above the first and the second threshold, the clusters include less than a predetermined number picture elements, are discarded.

According to still further features in the described preferred embodiments color or intensity coded saturation and/or blood volume maps are generated.

According to still further features in the described preferred embodiments the coded saturation maps are coded oxygen saturation maps.

According to still further features in the described preferred embodiments an anatomical image of the exposed cortex is generated and least one of the color or intensity coded saturation and/or blood volume maps and the anatomical image of the exposed cortex are co-displayed.

According to still further features in the described preferred embodiments the anatomical image is a monochromatic image.

According to still further features in the described preferred embodiments the anatomical image is a grayscale image.

According to still further features in the described preferred embodiments the anatomical image is a red-green-blue image.

According to still further features in the described preferred embodiments at least one of the color or intensity coded saturation and/or blood volume maps and the anatomical image of the exposed cortex are co-displayed side by side or superimposed.

According to still further features in the described preferred embodiments the image highlighting differences among spectra of the exposed cortex is coded via color or intensity so as to distinguish degree of the differences in accordance with at least one difference threshold.

According to still another aspect of the present invention there is provided a method of generating an oxygen saturation and/or blood volume difference map of a tissue of a subject, the method comprising the steps of (a) illuminating the tissue of the subject with incident light; (b) at a first time point, acquiring a spectrum of each picture element of the tissue of the subject; (c) at a second time point, acquiring at least one additional spectrum of each picture element of the tissue of the subject; and (d) generating an image highlighting differences among spectra of the tissue acquired in steps (b) and (c), so as to generate the oxygen saturation and/or blood volume difference map or a difference map of other substances of the tissue. Thresholds and other features as described above with respect to functional brain mapping are preferably applied in a similar manner.

The tissue can be any tissue, such as, but not limited to, a brain (for, but not limited to, monitoring neuronal activity), a heart (for, but not limited to, monitoring OS changes during bypass surgery, skin (for, but not limited to, monitoring OS changes following skin implantations, flaps, and other plastic surgery procedures), a liver, a kidney, an eye, and other applications where measuring concentrations of chemical compounds or changes in the concentrations in these compounds is of medical importance.

According to an additional aspect of the present invention there is provided a system of generating an oxygen saturation and/or blood volume difference map of a tissue of a subject, the system comprising (a) an illumination device for illuminating the tissue of the subject with incident light; (b) a spectral imaging device for acquiring spectra of each picture element of the tissue of the subject at a first time point and at a second time point; and (c) an image generating device for generating an image highlighting differences among spectra of the tissue acquired in the first and the second time points, so as to generate the oxygen saturation and/or blood volume difference map of the tissue.

According to yet an additional aspect of the present invention there is provided a system for monitoring oxygen saturation in a tissue comprising a spectral imaging device and an image generating device, the spectral imaging device and the image generating device acting in synergy to produce an oxygen saturation difference map by highlighting tissue regions characterized by (a) having an absolute or relative level of oxygen saturation above a predetermined first threshold; (b) having an oxygen saturation difference above a predetermined second threshold; and/or (c) having a cluster size above a predetermined size.

According to still an additional aspect of the present invention there is provided a system for monitoring blood volume in a tissue comprising a spectral imaging device and an image generating device, the spectral imaging device and the image generating device acting in synergy to produce a blood volume difference map by highlighting tissue regions characterized by (a) having an absolute or relative level of blood volume above a predetermined first threshold; (b) having a blood volume difference above a predetermined second threshold; and (c) having a cluster size above a predetermined size.

According to still an additional aspect of the present invention there is provided a system for functional brain mapping comprising a spectral imaging device and an image generating device, said spectral imaging device and said image generating device acting in synergy to produce an anatomical image of the brain or a portion thereof and a coded functional map of the brain or said portion thereof, said coded functional map reflecting a change in the brain in response to a stimulus, said functional map and the anatomical image being co-displayed.

According to another aspect of the present invention there is provided a method of brain mapping of a subject, in particular an epilepsy patient, comprising the steps of (a) illuminating an exposed cortex of a brain or portion thereof of the subject with incident light; (b) acquiring a reflectance spectrum of each picture element of at least a portion of the exposed cortex of the subject; and (e) generating an image highlighting concentrations of at least one substance in the brain.

The present invention successfully addresses the shortcomings of the presently known configurations by (i) enabling, for the first time, high spatial and spectral resolution functional brain mapping, which can be used to identify functional regions in the brain during a neurosurgery even in cases where the anatomy of the brain is vastly distorted; and (ii) providing a novel algorithm for determining oxygen saturation and/or blood volume differences in a tissue as a response to a stimulus, oxygenation, deoxygenation or blood perfusion.

Implementation of the methods and systems of the present invention involves performing or completing selected tasks or steps manually, automatically, or a combination thereof. Moreover, according to actual instrumentation and equipment of preferred embodiments of the method and system of the present invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computer using any suitable operating system. In any case, selected steps of the methods and systems of the invention could be described as being performed by a data processor, such as a computing platform for executing a plurality of instructions.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 shows the homunculus, a graphic projection of the human body in which organs are given a size proportional to the cortex area they occupy;

FIGS. 2a-f show the rough anatomy of the human cortex, highlighting the prefrontal area, frontal lobe, sensorimotor lobe, parietal lobe, occipital lobe and the temporal lobe, respectively.

FIGS. 3a-b show the sensory homunculus of the human brain, a graphic projection of the human body onto the surface of the sensory cortex of the brain, depicting the extent of the area nerving each part of the sensory portions of the body;

FIGS. 4a-b show the motor homunculus of the human brain, a graphic projection of the human body onto the surface of the motor cortex of the brain, depicting the extent of the area activating each part of the body subject to voluntary control;

FIG. 5 defines the superior temporal gyros (STG) area over a photograph of a human brain;

FIG. 6 is a schematic and simplified depiction of a system in accordance with the teachings of the present invention;

FIG. 7 is a block diagram illustrating the main components of an imaging spectrometer constructed in accordance with U.S. Pat. No. 5,539,517 (prior art), commercially available as SPECTRACUBE from Applied Spectral Imaging, Migdal Ha'Eemek, Israel;

FIG. 8 illustrates a non-moving type interferometer, namely, a Sagnac interferometer, as used in a spectral imaging device (imaging spectrometer) in accordance with U.S. Pat. No. 5,539,517 (prior art);

FIG. 9 is a schematic depiction of a filters-based spectral imaging device suitable to implement the methods of the present invention;

FIG. 10 shows a red-green-blue (RGB) image reconstructed from a spectral cube acquired on awake patient undergoing brain surgery using an imaging spectrometer of U.S. Pat. No. 5,539,517;

FIG. 11 shows hemoglobin absorption spectra, Hb—deoxy-hemoglobin, Hb-O₂—oxy-hemoglobin;

FIG. 12 demonstrates an example of filters selection for a filters-based spectral imaging device according to the present invention;

FIG. 13a presents a graph showing a typical normalized measured reflectance spectrum of the human cortex.

FIG. 13b demonstrates intensity results calculated using the mathematical filters shown in FIG. 12 to mathematically manipulate light derived from a representative picture element of the human cortex according to the present invention;

FIG. 13c is a graph showing an interpolation of the discrete spectrum, shown in 13b, using a spline method;

FIG. 13d is a graph showing the optical density spectrum of the spectrum presented in 13a, and the fit calculated for it using the method described herein;

FIG. 13e is a graph showing the optical density of the curve obtained by interpolating on filter-measured data (FIG. 13c) along with the fit calculated for it using the method described herein;

FIG. 13f is a graph showing calculated fits to the filter and spectral-imaging measured signals. The fits correlate with OS calculated values of 93% (when measured with spectral-imaging) and 88% (when measured with filters);

FIG. 14 is a Ti-weighted MRI image of a brain, copied from the Web site of Mayo clinic (USA), (http://www.mayo.edu/);

FIG. 15 is an fMRI image during photic stimulation, copied from the Web site of Mayo clinic (USA), (http://www.mayo.edu/);

FIG. 16 demonstrates masking the fMRI image with the Ti brain mask, copied from the Web site of Mayo clinic (USA), (http://www.mayo.edu/);

FIG. 17 shows an oxygen saturation difference map overlaid on a monochromatic gray-scale image of the cortex, highlighting pixels (in red) corresponding to brain regions (picture elements) that underwent an increase in oxygen saturation (OS) that reached an OS level greater then 40% and have risen by more then 1% post left palm electrical stimulation, and colored in yellow are pixels that reached an OS level greater then 40% and have risen by less than 1%, according to the present invention;

FIG. 18 shows an oxygen saturation difference map overlaid on a monochromatic gray-scale image of the cortex, highlighting pixels (in red) corresponding to brain regions (picture elements) that underwent an increase in oxygen saturation (OS) that reached an OS level greater then 45% and have risen by more then 1% post left palm electrical stimulation, and colored in yellow are pixels that reached an OS level greater then 45% and have risen by less than 1%, according to the present invention;

FIG. 19 shows an oxygen saturation difference map overlaid on a monochromatic gray-scale image of the cortex, highlighting pixels (in red) corresponding to brain regions (picture elements) that underwent an increase in oxygen saturation (OS) that reached an OS level greater then 50% and have risen by more then 1% post left palm electrical stimulation, and colored in yellow are pixels that reached an OS level greater then 50% and have risen by less than 1%, according to the present invention;

FIG. 20 shows an oxygen saturation difference map overlaid on a monochromatic gray-scale image of the cortex, highlighting pixels (in red) corresponding to brain regions (picture elements) that underwent an increase in oxygen saturation (OS) that reached an OS level greater then 55% and have risen by more then 1% post left palm electrical stimulation, and colored in yellow are pixels that reached an OS level greater then 55% and have risen by less than 1%, according to the present invention;

FIG. 21 shows an oxygen saturation difference map overlaid on a monochromatic gray-scale image of the cortex, highlighting pixels (in red) corresponding to brain regions (picture elements) that underwent an increase in oxygen saturation (OS) that reached an OS level greater then 60% and have risen by more then 1% post left palm electrical stimulation, and colored in yellow are pixels that reached an OS level greater then 60% and have risen by less than 1%, according to the present invention;

FIG. 22 shows an oxygen saturation difference map overlaid on a monochromatic gray-scale image of the cortex, highlighting pixels (in red) corresponding to brain regions (picture elements) that underwent an increase in oxygen saturation (OS) that reached an OS level greater then 65% and have risen by more then 1% post left palm electrical stimulation, and colored in yellow are pixels that reached an OS level greater then 65% and have risen by less than 1%, and colored in yellow are pixels that reached an OS level greater then 65% and have risen by less than 1%, according to the present invention;

FIG. 23 shows an oxygen saturation difference map overlaid on a monochromatic gray-scale image of the cortex, highlighting pixels (in red) corresponding to brain regions (picture elements) that underwent an increase in oxygen saturation (OS) that reached an OS level greater then 70% and have risen by more then 1% post left palm electrical stimulation, and colored in yellow are pixels that reached an OS level greater then 70% and have risen by less than 1%, according to the present invention;

FIG. 24 shows an oxygen saturation difference map overlaid on a monochromatic gray-scale image of the cortex, highlighting pixels corresponding to brain regions (picture elements) that underwent an increase in oxygen saturation (OS) that reached an OS level greater then 65% and have risen by more then 1% (red) or less than 1% (yellow) post left palm electrical stimulation, according to the present invention;

FIG. 25 shows an oxygen saturation difference map overlaid on a monochromatic gray-scale image of the cortex, highlighting pixels corresponding to brain regions (picture elements) that underwent an increase in oxygen saturation (OS) that reached an OS level greater then 65% and have risen by more then 3% (red) or less than 3% (yellow) post left palm electrical stimulation, according to the present invention;

FIG. 26 shows an oxygen saturation difference map overlaid on a monochromatic gray-scale image of the cortex, highlighting pixels corresponding to brain regions (picture elements) that underwent an increase in oxygen saturation (OS) that reached an OS level greater then 65% and have risen by more then 5% (red) or less than 5% (yellow) post left palm electrical stimulation, according to the present invention;

FIG. 27 shows an oxygen saturation difference map overlaid on a monochromatic gray-scale image of the cortex, highlighting pixels corresponding to brain regions (picture elements) that underwent an increase in oxygen saturation (OS) that reached an OS level greater then 65% and have risen by more then 10% (red) or less than 10% (yellow) post left palm electrical stimulation, according to the present invention;

FIG. 28 shows an fMRI image demonstrating the activation of Wernike's area;

FIG. 29 is a CT image showing a section of the brain, the tumor is clearly seen on the right-hand side (the left hemisphere);

FIG. 30 is a gray-scale orientation image as observed by the spectral imaging device constructed in accordance with U.S. Pat. No. 5,539,517;

FIG. 31 shows a color coded oxygen saturation map of a patient's cortex pre translation task, according to the present invention;

FIG. 32 shows a color coded oxygen saturation map of a patient's cortex post translation task, according to the present invention;

FIG. 33 shows an oxygen saturation difference map overlaid on a monochromatic gray-scale image of the cortex, highlighting pixels corresponding to brain regions (picture elements) that underwent an increase in oxygen saturation (OS) that reached an OS level greater then 90% and have risen by more then 5% (red) or less than 5% (yellow) post translation task, according to the present invention;

FIG. 34 shows a CT image showing a single tumor strand in left temporal area;

FIG. 35 shows an fMRI image showing dominant Broca.

FIG. 36 is a gray-scale orientation image as observed by the spectral imaging device constructed in accordance with U.S. Pat. No. 5,539,517;

FIG. 37 shows an oxygen saturation difference map overlaid on a monochromatic gray-scale image of the cortex, highlighting pixels corresponding to brain regions (picture elements) that underwent an increase in oxygen saturation (OS) that reached an OS level greater then 90% and have risen by more then 5% (red) or less than 5% (yellow), highlighting speech-associated areas, according to the present invention;

FIG. 38 shows an oxygen saturation difference map overlaid on a monochromatic gray-scale image of the cortex, highlighting pixels corresponding to brain regions (picture elements) that underwent an increase in oxygen saturation (OS) that reached an OS level greater then 90% and have risen by more then 5% (red) or less than 5% (yellow), highlighting right hand fingers movement associated areas, according to the present invention;

FIG. 39 shows an oxygen saturation difference map overlaid on a monochromatic gray-scale image of the cortex, highlighting pixels corresponding to brain regions (picture elements) that underwent an increase in oxygen saturation (OS) that reached an OS level greater then 90% and have risen by more then 5% (red) or less than 5% (yellow), highlighting mouth movement (open-close) associated areas, according to the present invention;

FIG. 40 shows a color coded oxygen saturation map of a patient's cortex pre passive optical stimulation, according to the present invention;

FIG. 41 shows a color coded oxygen saturation map of a patient's cortex post passive optical stimulation, according to the present invention;

FIG. 42 is a gray-scale orientation image as observed by the spectral imaging device constructed in accordance with U.S. Pat. No. 5,539,517;

FIG. 43 shows an oxygen saturation difference map overlaid on a monochromatic gray-scale image of the cortex, highlighting pixels corresponding to brain regions (picture elements) that underwent an increase in oxygen saturation (OS) that reached an OS level greater then 90% and have risen by more then 5% (red) or less than 5% (yellow) post passive optical stimulation, according to the present invention;

FIG. 44 shows an oxygen saturation map of a human cortex according to the present invention;

FIG. 45 shows a color coded blood volume map of the human cortex of FIG. 44 according to the present invention;

FIG. 46 demonstrates calculation of oxygen saturation based on spectral data collected via a spectral imaging device and known absorption spectra of hemoglobin according to the present invention;

FIG. 47 shows the generation of an oxygen saturation map according to the present invention;

FIG. 48 shows the generation of an oxygen saturation difference map overlaid on an anatomical image according to the present invention;

FIG. 49 shows the generation of an oxygen saturation map overlaid on an anatomical image according to the present invention;

FIG. 50 shows two labels placed on a skull around a craniotomy, the labels shown serve for image orientation with respect to the craniotomy, as white targets and as scales;

FIGS. 51a-e show a series of oxygen saturation difference maps each overlaid on a monochromatic gray-scale image of the cortex, highlighting pixels corresponding to brain regions (picture elements) that underwent an increase in oxygen saturation (OS) that reached an OS level greater then 90% and have risen by more then 5% (red) or less than 5% (yellow) post passive optical stimulation, according to the present invention, wherein the stimulations were flashing light into an eye of the patient (51a) and four independent stimulations with an opto-kinetic strip (51b-d); and

FIG. 52 is a cumulative differences map (display) of the oxygen saturation difference maps of FIGS. 51a-e, in accordance with the teachings of the present invention.

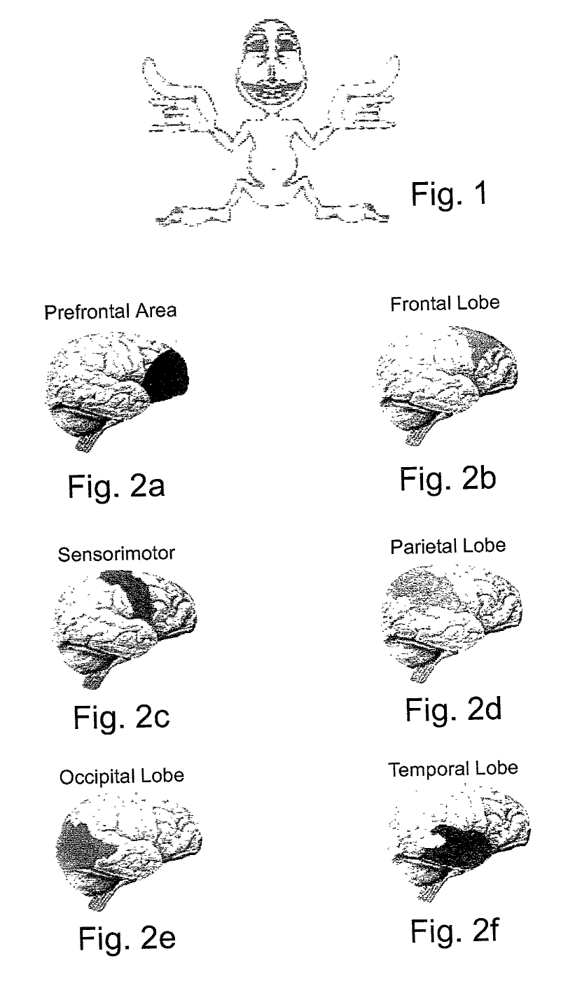

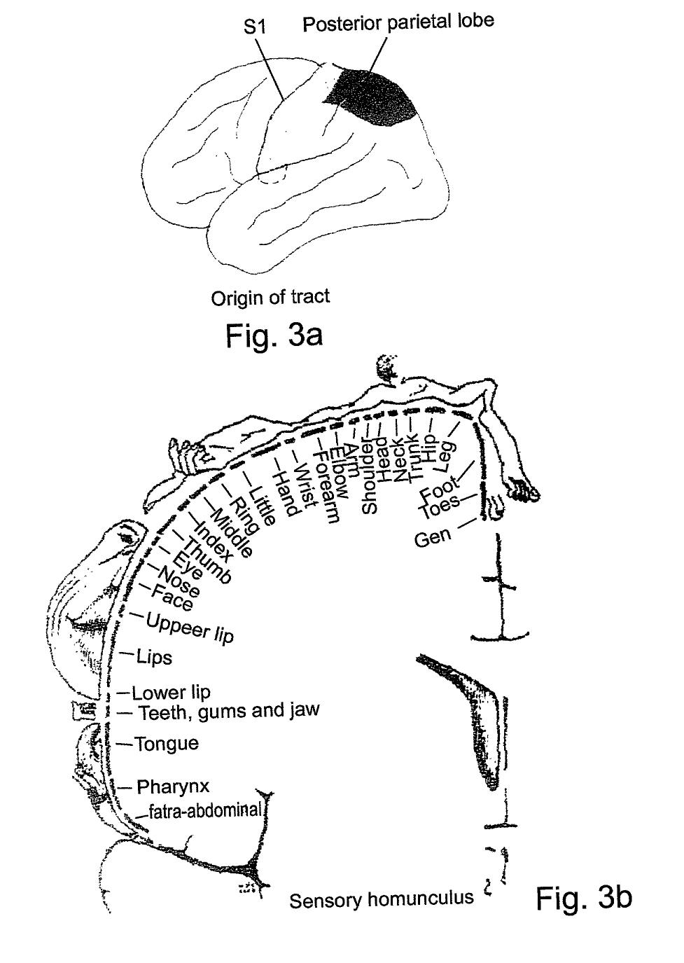

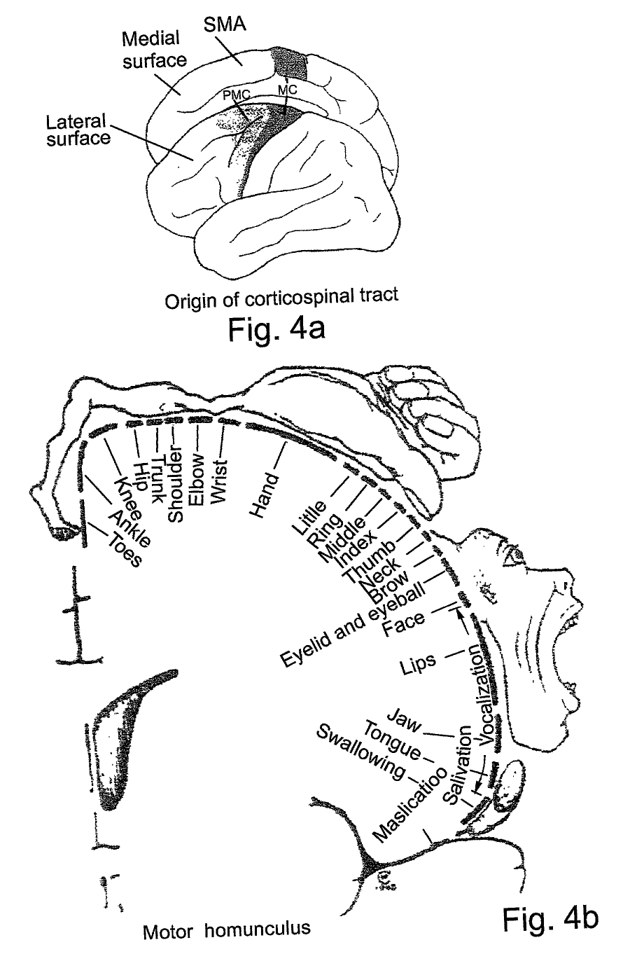

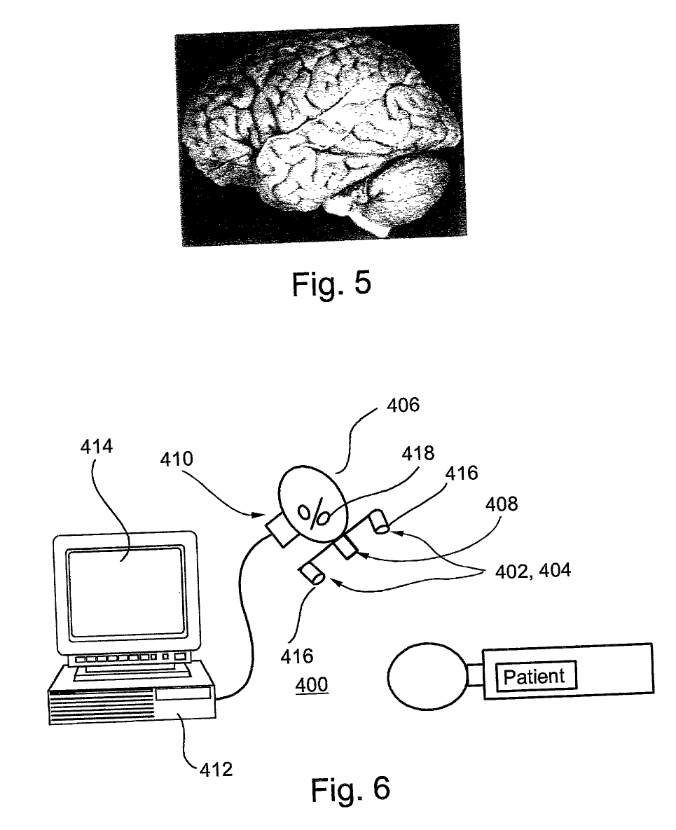

### DESCRIPTION OF THE PREFERRED EMBODIMENTS

The present invention is of systems and methods for functional brain mapping which can be used during neurosurgeries and further of novel oxygen saturation and/or blood volume maps and novel oxygen saturation and/or blood volume difference maps which can be used for effecting same. Specifically, the present invention can be used to acquire high spectral and spatial resolution spectral images of an exposed cortex during a neurosurgery, while using peripheral or direct, voluntary or passive, brain stimulation protocols for mapping functional cortical regions and thereby deducing cortical anatomy in real time, especially in cases of distorted anatomy, as is typically the case when a space-occupying lesion, e.g., a brain tumor, distorts neighboring brain tissue. Further specifically, the present invention can be used to generate and display oxygen saturation and/or blood volume maps and oxygen saturation and/or blood volume difference maps of any tissue, highlighting differences in the oxygen saturation and/or blood volume characterizing the tissue between two or more time points.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

According to one aspect of the present invention there is provided a method of functional brain mapping of a subject. The method according to this aspect of the invention is effected by implementing the following method steps. Throughout steps of spectral data acquisition, the cortex or a portion thereof is illuminated with incident light. The light can be white light or filtered light. The light is preferably a cold light. If hot light is employed, lighting durations should be minimized, so as to avoid heat induced damage to the exposed brain. Presently, without limitation, regulated halogen light is preferred. While lighting the cortex as described, a reflectance spectrum of each picture element of at least the portion of the exposed cortex is acquired. Thereafter, the brain is stimulated through, for example, the peripheral nervous system of the subject, and during and/or after the stimulation, at least one additional reflectance spectrum of each picture element of at least the portion of the exposed cortex is acquired. Finally, an image highlighting differences among spectra of the exposed cortex so as to highlight functional brain regions is generated.

The above method can be implemented while performing a neurosurgery for the removal of a mass (of tissue) from a brain of a subject while minimizing damage to a neighboring brain tissue. To this end, a craniotomy is performed so as to expose at least a portion of a cortex of the subject. Thereafter, functional brain mapping of the subject is performed essentially as described above, i.e., by (i) illuminating the exposed portion of the cortex with incident light; (ii) acquiring a reflectance spectrum of each picture element of at least a portion of the exposed cortex of the subject; (iii) stimulating the neighboring brain tissue of the subject (e.g., inducing brain activity); (iv) during or after step (iii) acquiring at least one additional reflectance spectrum of each picture element of at least the portion of the exposed cortex of the subject; and (v) generating an image highlighting differences among spectra of the exposed cortex acquired in steps (ii) and (iv), so as to highlight the functional brain regions of the neighboring brain tissue. Finally, assisted by the image, the mass is surgically removed while minimizing damage to the neighboring brain tissue.

The brain mass can be a brain tumor, either benign or malignant tumor, or the brain mass can be a brain tissue removed in order to treat neurologic (e.g., epilepsy) or phsichotic disorders (e.g., lobotomy).

As is shown in FIG. 6, according to another aspect of the present invention there is provided a system 400 for functional brain mapping of a subject. The system includes an illumination device 402 which serves for illuminating an exposed cortex or portion thereof with incident light. The illumination device may form an integral part of the system or may be a stand-alone device). Illumination device 402 preferably includes a plurality of individual light sources 404 arranged and directed so as to provide substantially homogenous lighting of the exposed cortex, each of which may include a wide band filter 416, which serves for restricting light wavelengths to a predetermined range, so as to reduce noise. System 400 further includes a spectral imaging device 406 which serves for acquiring reflectance spectra of each picture element of at least a portion of the exposed cortex before and during and/or after stimulating the brain (e.g., inducing brain activity). The optics of device 406 may vary as is further detailed hereinunder, however, in all of its configurations, device 406 includes an objective lens or other type of fore optics 408 which serves to direct light into device 406 and a light intensity recording device 410, such as a charge coupled device (CCD), which serves for data acquisition. Depending on the specific application, a wide band filter 418 can be used to restrict the wavelength of the incoming light as desired. System 400 further includes an image generating device 412 which serves for generating an image highlighting differences among spectra of the exposed cortex acquired before and during and/or after stimulating the brain of the subject, so as to highlight functional brain regions. Device 412 is typically connected to a display 414 which serves to display the results. Device 412 can be a suitable computer such as a personal computer equipped and designed to execute certain algorithms, which would result in generating and displaying an image highlighting differences among spectra of the exposed cortex acquired before and during and/or after stimulating the brain of the subject, so as to highlight functional brain regions. Being a computer, certain functions of device 406, which functions are related to data acquisition are also executed by device 412, although a separate computational platform can be used to this end. Thus, device 412, is preferably an integrated device which is used for performing a number of tasks related to both spectral imaging data acquisition per se and to the analysis and presentation of the results thereof.

The following provides several alternative configurations for spectral imaging device 406, one alternative relates to interferometer-based spectral imaging devices, whereas the other relates to filters-based spectral imaging devices.

### Interferometer-based Spectral Imaging Devices

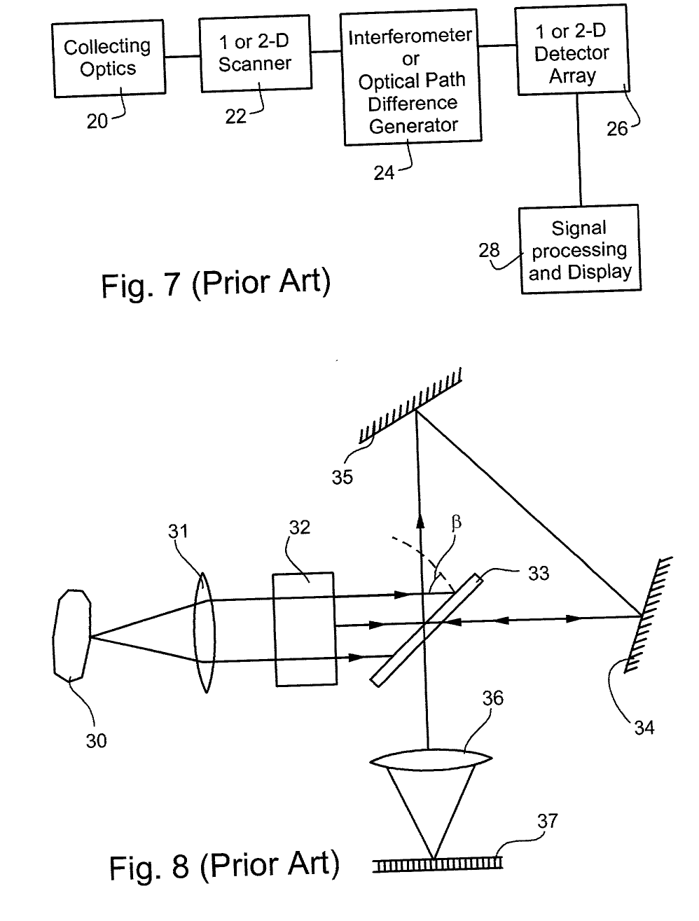

FIG. 7 is a block diagram illustrating the main components of a prior art imaging spectrometer disclosed in U.S. Pat. No. 5,539,517, which is incorporated by reference as if fully set forth herein.

This imaging spectrometer is constructed highly suitable to implement the method of the present invention as it has high spectral (Ca. 4-14 nm depending on wavelength) and spatial (Ca. system MTF (modulation transfer function, e.g., 30)/M μm, where M is the effective fore optics magnification) resolutions.

Thus, the prior art imaging spectrometer of FIG. 6 includes: a collection optical system, generally designated 20; a one-dimensional scanner, as indicated by block 22; an optical path difference (OPD) generator or interferometer, as indicated by block 24; a one-dimensional or two-dimensional detector array, as indicated by block 26; and a signal processor and display, as indicated by block 28.

A critical element in system 20 is the OPD generator or interferometer 24, which outputs modulated light corresponding to a predetermined set of linear combinations of the spectral intensity of the light emitted from each picture element of the scene to be analyzed. The output of the interferometer is focused onto the detector array 26. Thus, all the required optical phase differences are scanned simultaneously for all the picture elements of the field of view, in order to obtain all the information required to reconstruct the spectrum. The spectra of all the picture elements in the scene are thus collected simultaneously with the imaging information, thereby permitting analysis of the image in a real-time manner.

The apparatus according to U.S. Pat. No. 5,539,517 may be practiced in a large variety of configurations. Specifically, the interferometer used may be combined with other mirrors as described in the relevant Figures of U.S. Pat. No. 5,539,517.

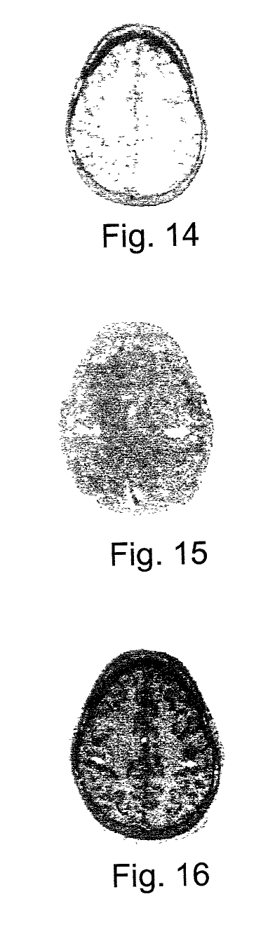

Thus, alternative types of interferometers may be employed. These include (i) a moving type interferometer in which the OPD is varied to modulate the light, namely, a Fabry-Perot interferometer with scanned thickness; (ii) a Michelson type interferometer which includes a beamsplitter receiving the beam from an optical collection system and a scanner, and splitting the beam into two paths; (iii) a Sagnac interferometer optionally combined with other optical means in which interferometer the OPD varies with the angle of incidence of the incoming radiation, such as the four-mirror plus beamsplitter interferometer as further described in the cited U.S. patent (see FIG. 14 there).

FIG. 8 illustrates an imaging spectrometer constructed in accordance with U.S. Pat. No. 5,539,517, utilizing an interferometer in which the OPD varies with the angle of incidence of the incoming radiation. A beam entering the interferometer at a small angle to the optical axis undergoes an OPD which varies substantially linearly with this angle.

In the interferometer of FIG. 8, all the radiation from source 30 in all the picture elements, after being collimated by an optical collection system 31, is scanned by a mechanical scanner 32. The light is then passed through a beamsplitter 33 to a first reflector 34 and then to a second reflector 35, which reflects the light back through the beamsplitter 33 and then through a focusing lens 36 to an array of detectors 37 (e.g., a CCD). This beam interferes with the beam which is reflected by 33, then by second reflector 35, and finally by first reflector 34.

At the end of one scan, every picture element has been measured through all the OPD's, and therefore the spectrum of each picture element of the scene can be reconstructed by Fourier transformation. A beam parallel to the optical axis is compensated, and a beam at an angle (θ) to the optical axis undergoes an OPD correction, which is a function of the thickness of the beamsplitter 33, its index of refraction, and the angle θ. The OPD is proportional to θ for small angles. By applying the appropriate inversion, and by careful bookkeeping, the spectrum of every picture element is calculated.

In the configuration of FIG. 8 the ray which is incident on the beamsplitter at an angle β (β=45° in FIG. 8) goes through the interferometer with an OPD=0, whereas a ray which is incident at a general angle β-θ undergoes an OPD given by Equation (1):

OPD(β,θ,t,n)=t[(n²−sin²(β+θ))^{0.5}−(n²−sin²(β−θ))^{0.5+2} sin β sin θ] (1)

where θ is the angular distance of a ray from the optical axis or interferometer rotation angle with respect to the central position; t is the thickness of the beamsplitter; and n is the index of refraction of the beamsplitter.

It follows from the above equation that by scanning both positive and negative angles with respect to the central position, one gets a double-sided interferogram for every picture element, which helps eliminate phase errors giving more accurate results in the Fourier transform calculation. The scanning amplitude determines the maximum OPD reached, which is related to the spectral resolution of the measurement. The size of the angular steps determines the OPD step which is, in turn, dictated by the shortest wavelength to which the system is sensitive. In fact, according to the sampling theorem [see, Chamberlain (1979) The principles of interferometric spectroscopy, John Wiley and Sons, pp. 53-55], this OPD step must be smaller than half the shortest wavelength to which the system is sensitive.

Another parameter which should be taken into account is the finite size of a detector element in the matrix. Through the focusing optics, the element subtends a finite OPD in the interferometer which has the effect of convolving the interferogram with a rectangular function. This brings about, as a consequence, a reduction of system sensitivity at short wavelengths, which drops to zero for wavelengths equal to or below the OPD subtended by the element. For this reason, one must ensure that the modulation transfer function (MTF) condition is satisfied, i.e., that the OPD subtended by a detector element in the interferometer must be smaller than the shortest wavelength at which the instrument is sensitive.

Thus, imaging spectrometers constructed in accordance with the invention disclosed in U.S. Pat. No. 5,539,517 do not merely measure the intensity of light coming from every picture element in the field of view, but also measure the spectrum of each picture element in a predefined wavelength range. They also better utilize all the radiation emitted by each picture element in the field of view at any given time, and therefore permit, as explained above, a significant decrease in the frame time and/or a significant increase in the sensitivity of the spectrometer. Such imaging spectrometers may include various types of interferometers and optical collection and focusing systems, and may therefore be used in a wide variety of applications.

An imaging spectrometer in accordance with the invention disclosed in U.S. Pat. No. 5,539,517 was developed by Applied Spectral Imaging Ltd., Industrial Park, Migdal Haemek, Israel and is referred to herein as SPECTRACUBE. This spectral imaging device was used to reduce the present invention to practice, yielding unexpected results as is further demonstrated in the Examples section that follows.

The SPECTRACUBE system has the following or better characteristics, listed hereinbelow in Table 1:

TABLE 1

| Parameter | Performance |
|---|---|
| Spatial resolution: | MTF/M μm (M = effective fore optics |
| | magnification) |
| Field of View: | 8.3/M millimeters |
| Sensitivity: | 20 milliLux (for 100 msec integration |
| | time, increases for longer integration |
| | times linearly with \sqrt{T}) |
| Spectral range: | 400-1000 nm |
| Spectral resolution: | 4 nm at 400 nm (16 nm at 800 nm) |
| Acquisition time: | 5-50 sec, typical 20 seconds |
| FFT processing time: | 5-60 sec, typical 20 seconds |

### Other Spectral Imaging Devices

The SPECTRACUBE system optically connected to a suitable fore optics is preferably used to analyze tissue, such as brain tissue, according to the methods of the present invention. However, any spectral imaging device, i.e., an instrument that measures and stores in memory for later retrieval and analysis the spectrum of light emitted by every point of an object which is placed in its field of view, including filter (e.g., conventional interference filters, acousto-optic tunable filters (AOTF) or liquid-crystal tunable filter (LCTF)) and dispersive (monochromator) element (e.g., grating or prism) based spectral imaging devices, or other spectral data or multi-band light collection devices (e.g., a device in accordance with the disclosure in Speicher R. M., Ballard S. G. and Ward C. D. (1996) Karyotyping human chromosomes by combinatorial multi-flour FISH. Nature Genetics, 12:368-375) can potentially be used to acquire the required spectral data. Also a device including a plurality of wide-band of (fixed or tunable) filters, as described in U.S. Pat. No. 5,834,203, and is incorporated by reference as if fully set forth herein, can be used as the spectral data collection device according to the present invention. Therefore, it is intended not to limit the scope of the present invention for use of any specific type of spectral imaging device.

### Interference Filters-based Spectral Imaging Devices

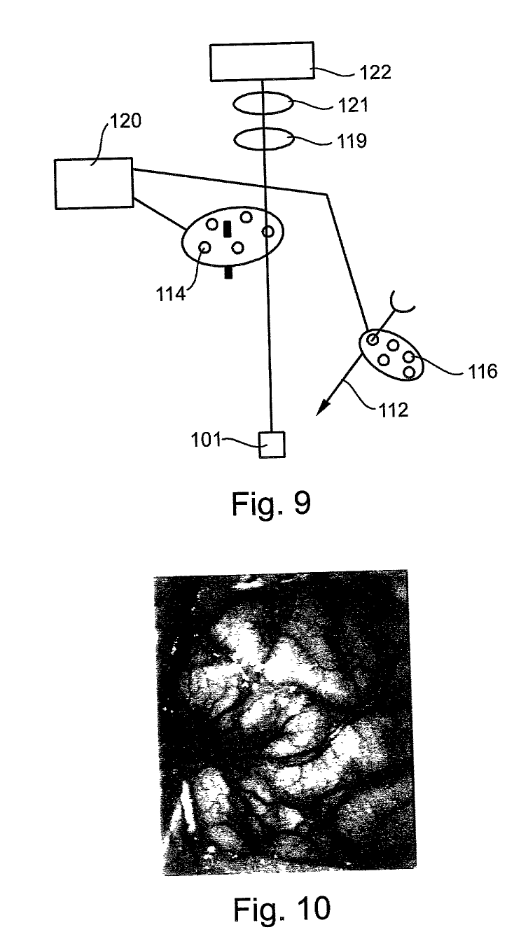

With reference now to FIG. 9. A filters-based spectral imaging device is referred to herein as apparatus 100 and includes an objective or fore optics 101. Apparatus 100 further includes a plurality of interference filters 114, five are shown. The filters are selected according to the features described hereinunder. Illumination filters 116 may also be employed, so as to restrict the illumination provided by a light beam 112 to specific wavelengths.

Apparatus 100 further includes an automatic, manual or semimanual control device 120. Device 120 serves for selecting among filters 114 and/or 116.

Apparatus 100 further includes a light intensity recording device 122 (e.g., a CCD) which serves for recording reflected light intensity as retrieved after passing through any one of filter 114.

As a result, each of the picture elements in the analyzed sample is representable by a vector of a plurality of dimensions, the number of dimensions being equal to the number of filters 114.

In a preferred embodiment apparatus 100 further includes a collimating lens 119 to ensure full collimation of the light before reaching recording device 122.

In a preferred embodiment apparatus 100 further includes a focusing lens 121 for focusing light reaching recording device 122.

The following provides considerations relating to filters 114 employed with apparatus 100.

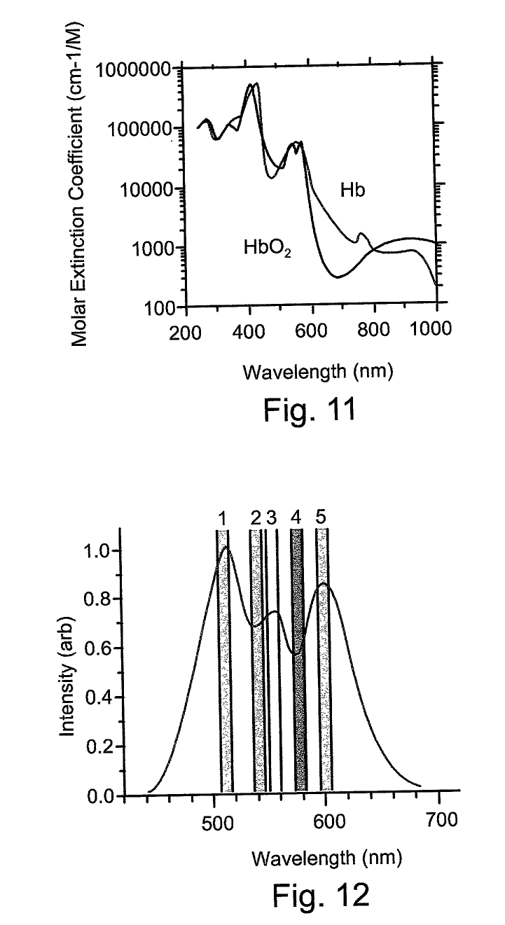

Thus, according to a preferred embodiment of the present invention the filters are selected so as to collect spectral data of intensity peaks and/or steeps characterizing one or more spectrally monitored substances, such as, but not limited to, peaks or steeps characterizing deoxy-hemoglobin, oxy-hemoglobin or deoxy-hemoglobin and oxy-hemoglobin. The different hemoglobin absorption spectra are shown in FIG. 11. Alternatively, filters are selected so as to collect spectral data of intensity peaks and/or steeps characterizing a single or an averaged picture element of the sample analyzed, e.g., the cortex. In any case, the normalized intensities measured using each of the discrete filters can be used as input for the algorithm of the present invention which is further described hereinunder. Thus, choice of filters is dictated by the spectral qualities one wishes to capture. The exact wavelength in which these phenomena will be detected (such as the double-peak of oxy-hemoglobin absorption, see FIG. 11) will differ from system to system as a function of the system response. The response is composed of the CCD quantum efficiency curve, the illumination curve and the transmittance curve of the system optics.

Herein, as shown in FIG. 12, five filters were selected based on spectral qualities of a representative spectrum of a picture element of the human cortex. Each filter is a 10 nm full-width-at-half-maximum (FWHM) filter, with high transmittance properties. Filters 1 and 5 are selected to collect spectral data from the peaks of the representative spectrum of the human cortex which peaks are at 513 nm and 600 nm. Filters 2 and 4 are selected to collect spectral data from the steeps of the representative spectrum of the human cortex which steeps are at 540 nm and 575 nm. Filter 3 is selected to collect spectral data from the minor peak of the representative spectrum of the human cortex which is attributed to oxy-hemoglobin and is at 555 nm.

Thus, according to preferred embodiments of the invention, each of the filters is individually about 5 to about 15 nm, preferably about 10 nm, full-width-at-half-maximum filter. The filters-based spectral imaging device of the invention may thus include N filters selected from the group consisting of an about 540 nm maximal transmittance filter, an about 575 nm maximal transmittance filter, an about 555 nm maximal transmittance filter, an about 513 nm maximal transmittance filter and an about 600 nm maximal transmittance filter, whereas N is an integer selected from the group consisting two, three, four and five. It will be appreciated that multiple chroic filter, such as dichroic filter or trichroic filter can replace a pair or triad of monochroic filters.

It will further be appreciated that different choices of filters are reasonable as well.

Thus, another optional choice for selecting filters for the spectral imaging device of the invention is setting two filters on two isosbasthic points (wavelengths where the absorption coefficients of oxy- and deoxy-hemoglobin coincide) and setting an additional (or more) filter(s) on a point showing great difference in the absorption values (see FIG. 11). Using this method requires performing a calibration for correlating the changes observed by the system to changes in oxygen saturation values.

Thus, according to this embodiment the filters include at least one, preferably several, say 2-5, filters of maximal transmittance at a wavelength which corresponds to at least one isosbasthic point of deoxy-hemoglobin and oxy-hemoglobin and at least one additional filter of maximal transmittance at a wavelength which corresponds to at least one, preferably several, say 2-5, non-isosbasthic points of deoxy-hemoglobin and oxy-hemoglobin.

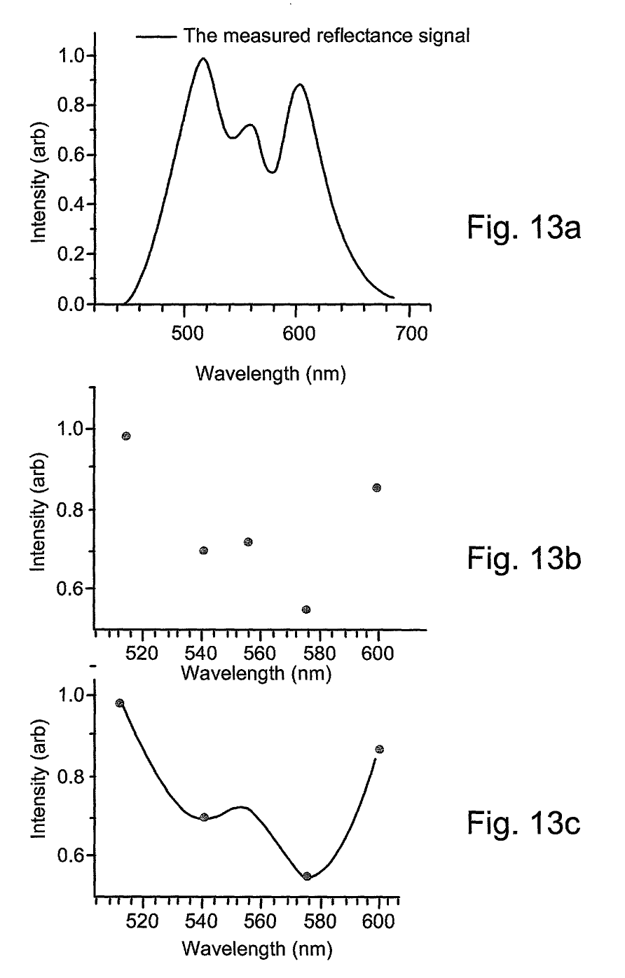

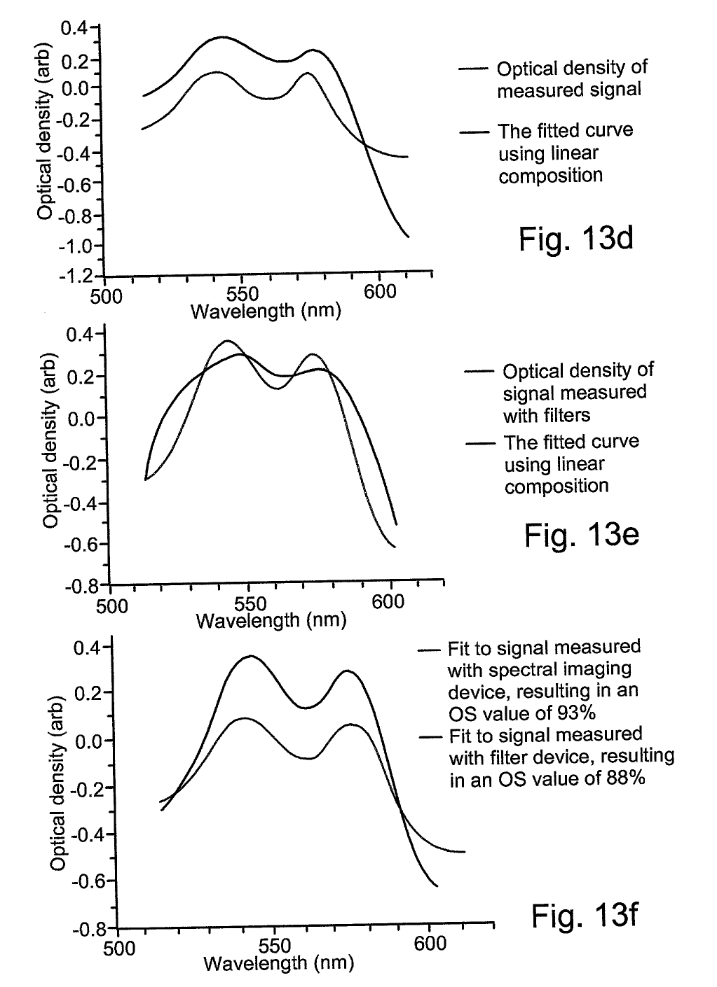

FIG. 13a is a graph showing a typical normalized measured reflectance spectrum of a picture element of the cortex.

FIG. 13b shows intensity results calculated using the mathematical filters shown in FIG. 12 to mathematically filter light derived from the representative picture element of the human cortex shown in FIG. 13a.

FIG. 13c is a graph showing an interpolation of the discrete spectrum using the spline method.

FIG. 13d demonstrates the optical density of the curve of FIG. 13a (in blue) and next to it (in red) the graph created by reconstructing a spectrum using the results obtained by mathematical manipulation using the method described below for determining oxygen saturation.

FIG. 13e shows, in blue, the optical density of the curve obtained by interpolating on filter-measured data (FIG. 13c) along with the fit (in red) calculated for it by reconstructing a spectrum using the results obtained by mathematical manipulation using the method described below for determining oxygen saturation.

FIG. 13f shows the calculated curves obtained by using the OS calculation method described below when applied to the spectrum measured by the interferometer system (FIG. 13a) and by mathematically extracting a filter-based spectrum (FIG. 13c). The fits correlate with OS calculated values of 93% (when measured with spectral-imaging) and 88% (when measured with filters).

### Analyzing and Displaying Spectral Imaging Data

### General Considerations and Approaches

General:

A spectral image is a three dimensional array of data, I(x,y,λ), that combines spectral information with spatial organization of the image. As such, a spectral image is a set of data called a spectral cube, due to its dimensionality, which enables the extraction of features and the evaluation of quantities that are difficult, and in some cases even impossible, to obtain otherwise. Since both spectroscopy and digital image analysis are well known fields that are covered by an enormous amount of literature [see, for example, Jain (1989) Fundamentals of Digital Image Processing, Prentice-Hall International], the following discussion will focus primarily on the benefit of combining spectroscopic and imaging information in a single data set, i.e., a spectral cube. Such a spectral cube of data can be collected by any spectral imaging device as is further delineated hereinabove.

One possible type of analysis of a spectral cube is to use spectral and spatial data separately, i.e., to apply spectral algorithms to the spectral data and two-dimensional image processing algorithms to the spatial data.

As an example of a spectral algorithm, consider an algorithm computing the similarity between a reference spectrum and the spectra of all pixels (i.e., similarity mapping) resulting in a gray (or other color) scale image (i.e., a similarity map) in which the intensity at each pixel is proportional to the degree of ‘similarity’. This gray scale image can then be further analyzed using image processing and computer vision techniques (e.g., image enhancement, pattern recognition, etc.) to extract the desired features and parameters. In other words, similarity mapping involves computing the integral of the absolute value of the difference between the spectrum of each pixel of the spectral image with respect to a reference spectrum (either previously memorized in a library, or belonging to a pixel of the same or other spectral image), and displaying a gray level or pseudocolor (black and white or color) image, in which the bright pixels correspond to a small spectral difference, and dark pixels correspond to a large spectral difference, or vice versa.

Similarly, classification mapping perform the same calculation as described for similarity mapping, yet takes several spectra as reference spectra, and paints each pixel of the displayed image with a different predetermined pseudocolor, according to its classification as being most similar to one of the several reference spectra.

It is also possible to apply spectral image algorithms based on non-separable operations; i.e., algorithms that include both local spectral information and spatial correlation between adjacent pixels (one of these algorithms is, as will be seen below, a principal component analysis).

One of the basic needs that arise naturally when dealing with any three-dimensional (3D) data structure such as a spectral cube (i.e., I(x,y,λ)), is visualizing that data structure in a meaningful way. Unlike other types of 3D data such as topographic data, D(x,y,z), obtained for example by a confocal microscope, where each point represents, in general, the intensity at a different location (x,y,z) in a tree-dimensional space, a spectral image is a sequence of images representing the intensity of the same two-dimensional plane (i.e., the sample) at different wavelengths. For this reason, the two most intuitive ways to view a spectral cube of data is to either view the image plane (spatial data) or the intensity of one pixel or a set of pixels as function of wavelength in a three-dimensional mountain-valley display. In general, the image plane can be used for displaying either the intensity measured at any single wavelength or the gray scale image that results after applying a spectral analysis algorithm, over a desired spectral region, at every image pixel. The spectral axis can, in general, be used to present the resultant spectrum of some spatial operation performed in the vicinity of any desired pixel (e.g., averaging the spectrum).

It is possible, for example, to display the spectral image as a gray scale image, similar to the image that might be obtained from a simple monochrome camera, or as a multicolor image utilizing one or several artificial colors to highlight and map important features. Since such a camera simply integrates the optical signal over the spectral range (e.g., 400 nm to 760 mn) of the CCD array, the ‘equivalent’ monochrome CCD camera image can be computed from the 3D spectral image data base by integrating along the spectral axis, as follows:

$$gray\_scale ( x , y ) = {\int }_{λ 1}^{λ2} w ( λ ) \cdot I ( x , y , λ ) ⅆ λ \qquad ( 2 )$$

In equation 2, w(λ) is a general weighting response function that provides maximum flexibility in computing a variety of gray scale images, all based on the integration of an appropriately weighted spectral image over some spectral range. For example, by evaluating equation 2 with three different weighting functions, {wᵣ(λ), w_{g}(λ), w_{b}(λ)}, corresponding to the tristimulus response functions for red (R), green (G) and blue (B), respectively, it is possible to display a conventional RGB color image. It is also possible to display meaningful non-conventional (pseudo) color images. FIG. 10 presents an example of the power of this simple algorithm. Consider choosing {wᵣ, w_{g}, w_{b}} to be Gaussian functions distributed “inside” a spectrum of interest, the resulting pseudo-color image that is displayed in this case emphasizes only data in the spectral regions corresponding to the weighting functions, enabling spectral differences in these three regions to be detected more clearly.

Point Operations:

Point operations are defined as those that are performed on single pixels, (i.e., do not involve more than one pixel at a time). For example, in a gray scale image, a point operation can be one that maps the intensity of each pixel (intensity function) into another intensity according to a predetermined transformation function. A particular case of this type of transformation is the multiplication of the intensity of each pixel by a constant. Additional examples include similarity and classification mapping as described hereinabove.

The concept of point operations can also be extended to spectral images: here each pixel has its own intensity function (spectrum), i.e., an n-dimensional vector V₁(λ); λε[λ₁, λₙ]. A point operation applied to a spectral image can be defined as one that maps the spectrum of each pixel into a scalar (i.e., an intensity value) according to a transformation function:

ν₂=g(V₁(λ)); λε[λ₁, λₙ] (3)

Building a gray scale image according to Equation 3 is an example of this type of point operation. In the more general case, a point operation maps the spectrum (vector) of each pixel into another vector according to a transformation function:

V₂(l)=g(V₁(λ)); 1ε[1, N], λε[λ₁, λₙ] (4), where N≦n.

In this case a spectral image is transformed into another spectral image.

One can now extend the definition of point operations to include operations between corresponding pixels of different spectral images. An important example of this type of algorithm is optical density analysis. Optical density is employed to highlight and graphically represent regions of an object being studied spectroscopically with higher dynamic range than the transmission spectrum. The optical density is related to transmission by a logarithmic operation and is therefore always a positive function. The relation between the optical density and the measured spectra is given by Lambert Beer law:

$$OD ( λ ) = - {log}_{10} \frac{I ( λ )}{{I}_{0} ( λ )} = - {log}_{10} τ ( λ ) \qquad ( 5 )$$

where OD(λ) is the optical density as a function of wavelength, I(λ) is the measured spectrum, Iₒ(λ) is a measured reference spectrum, and τ(λ) is the spectral transmittance of the sample. Equation 5 is calculated for every pixel for every wavelength where Iₒ(λ) is selected from (1) a pixel in the same spectral cube for which OD is calculated; (2) a corresponding pixel in a second cube; and (3) a spectrum from a library.

Note that the optical density does not depend on either the spectral response of the measuring system or the non-uniformity of the CCD detector. This algorithm is useful to map the relative concentration, and in some cases the absolute concentration of absorbers in a sample, when their absorption coefficients and the sample thickness are known.

Additional examples include various linear combination analysis, such as, but not limited to, (i) applying a given spectrum to the spectrum of each of the pixels in a spectral image by an arithmetical function such as addition, subtraction, multiplication division and combinations thereof to yield a new spectral cube, in which the resulting spectrum of each pixel is the sum, difference, product ratio or combination between each spectrum of the first cube and the selected spectrum; and (ii) applying a given scalar to the spectra of each of the pixels of the spectral image by an arithmetical function as described above.

Such linear combinations may be used, for example, for background subtraction in which a spectrum of a pixel located in the background region is subtracted from the spectrum of each of the pixels; and for a calibration procedure in which a spectrum measured prior to sample analysis is used to divide the spectrum of each of the pixels in the spectral image.

Another example includes a ratio image computation and display as a gray level image. This algorithm computes the ratio between the intensities at two different wavelengths for every pixel of the spectral image and paints each of the pixels in a lighter or darker artificial color accordingly. For example, it paints the pixel bright for high ratio, and dark for low ratio (or the opposite), to display distributions of spectrally sensitive materials.

Spatial-spectral Combined Operations:

In all of the spectral image analysis methods mentioned above, algorithms are applied to the spectral data. The importance of displaying the spectrally processed data as an image is mostly qualitative, providing the user with a useful image. It is also possible, however, depending on the application, to use the available imaging data in even more meaningful ways by applying algorithms that utilize the spatial-spectral correlation that is inherent in a spectral image. Spatial-spectral operations represent the most powerful types of spectral image analysis algorithms. As an example, consider the following situation:

A sample contains k cell types stained with k different stains (the term ‘cell’ here is used both for a biological cell, and also as ‘a region in the field of view of the instrument’). Each stain has a distinct spectrum and binds to only one of the k cell types. It is important to find the average intensity per cell for each one of the k cell types. To achieve this task the following procedure can be used: (i) classify each pixel in the image as belonging to one of k+1 classes (k cell types plus a background) according to its spectrum; (ii) segment the image into the various cell types and count the number of cells from each type; and (iii) sum the spectral energy contributed by each class, and divide it by the total number of cells from the corresponding class.

This procedure makes use of both spectral and spatial data. The relevant spectral data takes the form of characteristic cell spectra (i.e., spectral “signatures”), while the spatial data consists of data about various types of cells (i.e., cell blobs) many of which appear similar to the eye. In the above situation, cells can be differentiated by their characteristic spectral signature. Hence, a suitable point operation will be performed to generate a synthetic image in which each pixel is assigned one of k+1 values. Assuming that the spectra of the different cell types are known to be sᵢ(λ); i=1, 2 . . . , k, λε[λ₁, λₙ], and the measured spectrum at each pixel (x, y) is S_{x,y}(λ), λε[λ₁, λₙ], then the following algorithm is a possible method of classification (step 1 above):

Let e²ᵢ be the deviation of the measured spectrum from the known spectrum of the stain attached to cell type i. Then, adopting a least-squares “distance” definition, one can write:

$${e}_{i}^{2} = {\sum }_{λ ∈ {R}_{λ}}^{} {( s ( λ ) - {s}_{i} ( λ ) )}^{2} \qquad ( 6 )$$

where R_{λ} is the spectral region of interest. Each point [pixel (x, y)] in the image can then be classified into one of the k+1 classes using the following criterion: point(x,y) ε class k+1 if e²ᵢ > threshold for all i ε[1,k],

whereas (7)

point(x,y) ε class ρ if e²ᵢ< threshold, and ρ is such that min[e²ᵢ]=e²_{ρ}.

Steps ii and iii above (image segmentation and calculation of average intensity) are now straight-forward using standard computer vision operations on the synthetic image created in accordance with the algorithm described in equations 6 and 7.

Another approach is to express the measured spectrum s_{x,y}(λ) at each pixel as a linear combination of the k known fluorescence spectra sᵢ(λ); i=1, 2, . . . , k. In this case one would find the coefficient vector C=[c₁, c₂, . . . cₖ] that solves:

$$F = min {\sum }_{λ ∈ {R}_{λ}} {( s ( λ ) - \hat{s} ( λ ) )}^{2} \\ where \hat{s} ( λ ) = {\sum }_{i = 1}^{k} {c}_{i} \cdot {s}_{i} ( λ ) , \qquad ( 8 )$$

Solving for

$$\frac{ⅆ F}{ⅆ {c}_{i}} = 0 ;$$

for i=1, 2, . . . , k (i.e., find values of cᵢ which minimize F) yields the matrix equation C=A⁻¹B (9), where A is a square matrix of dimension k with elements

$${a}_{m , n} = [ {\sum }_{λ ∈ {R}_{λ}}^{} {s}_{m} ( λ ) \cdot {s}_{n} ( λ ) ] , \qquad ( 10 )$$

and B is a vector defined as

$${b}_{m} = [ {\sum }_{λ ∈ {R}_{λ}} {s}_{m} ( λ ) \cdot s ( λ ) ] , m , n = 1 , 2 , … , k . \qquad ( 11 )$$

Arithmetic operations may similarly be applied to two or more spectral cubes and/or spectra of given pixels or from a library. For example consider applying an arithmetic operations between corresponding wavelengths of corresponding pairs of pixels belonging to a first spectral cube of data and a second spectral cube of data to obtain a resulting third spectral cube of data for the purpose of, for example, averaging two spectral cubes of data, time changes follow-up, spectral normalization, etc.

In many cases objects (e.g., cells) present in a spectral image differ from one another in chemical constituents and/or structure to some degree, especially when stained. Using a decorrelation analysis, such as a principal component analysis, by producing covariance or a correlation matrix, enhances these differences. Decorrelation statistical analysis is directed at extracting decorrelated data out of a greater amount of data, and average over the correlated portions thereof There are a number of related statistical decorrelation methods. Examples include but not limited to principal component analysis (PCA), canonical variable analysis and singular value decomposition, etc., of these methods PCA is perhaps the more common one, and is used according to the present invention for decorrelation of spectral data, as this term is defined above. However, considering the fact that all decorrelation statistical methods including those listed above are related to one another, there is no intention to limit the scope of the invention to use of any specific decorrelation method. Specifically, there is no intention to limit the scope of the present invention to use of principal component analysis, as any other decorrelation statistical method may be alternatively employed. Information concerning the use and operation of the above listed decorrelation statistical methods is found in R. A. Johnson and D. W. Wichen, “Applied Multivariance Statistical Analysis, third edition, Prentice Hall (1992) and T. W. Anderson, An Introduction to Multivariance Statistical Analysis, second edition, Wiley and Sons (1984), both are incorporated by reference as if fully set forth herein.

Furthermore, as will become apparent from the descriptions to follow, the implementation of a decorrelation statistical method may be done using various modifications. As the concept of the present invention is not dependent upon any specific modification, it is the intention that the scope of the present invention will not be limited to any specific modification as described below.

A brief description of the principal component analysis using a covariance matrix is given below. For further details regarding the principal component analysis, the reader is referred to Martens and Naes (1989) Multivariate Calibration, John Wiley & Sons, Great Britain; and to Esbensen et al., Eds. (1994) Multi variance analysis—in practice. Computer-aided modeling as CAMO, and the Unscrambler's User's guide, Trondheim, Norway.

Thus, the intensities of the pixels of the image at wavelength λᵢ (i=1, . . . N) are now considered a vector whose length is equal to the number of pixels q. Since there are N of these vectors, one for every wavelength of the measurement, these vectors can be arranged in a matrix B′ with q rows, and N columns:

$${B}^{′} = No . of pixels \begin{matrix} & No . of wavelengths & \\ {B}_{11}^{′ } & … & {B}_{1 N}^{′} \\ ⋮ & & ⋮ \\ {B}_{q1}^{′} & … & {B}_{qN}^{′}\end{matrix} \qquad ( 12 )$$

For each of the columns of matrix B′ defined is an average:

$${M}_{i =} \frac{1}{q} {\sum }_{i = 1}^{q} {B}_{ji}^{′} ; i = 1 … N \qquad ( 13 )$$

and a second normalized matrix B defined as:

$$B = No . of pixels \begin{matrix} & No . of wavelengths & \\ {B}_{11}^{′} / {M}_{1} & … & {B}_{1 N}^{′} / {M}_{N} \\ ⋮ & & ⋮ \\ {B}_{q1}^{′} / {M}_{1} & … & {B}_{qN}^{′} / {M}_{N}\end{matrix} \qquad ( 14 )$$

A covariance matrix C is defined for the matrix B: C=B^{T}·B of dimensions N×N. C is diagonalized, and eigenvectors and eigenvalues related by: C·Vi=μᵢ·Vᵢ where Vᵢ are N orthogonal unit vectors and μᵢ are the eigenvalues representing the variance in the direction of the i-th unit vector Vᵢ. In general, the lowest components represent the highest variability as a function of pixels.

The products BVᵢ (i=1, . . . N) are the projections of the spectral image onto the elements of the orthogonal basis, They are vectors with q elements (q=number of pixels), and can be displayed separately as black and white images. These images may reveal features not obvious from a regular black and white image filtered at a certain wavelength or wavelength range.

### Spatial and Spectral Resolutions

According to a preferred embodiment of the present invention each spectral data collection step of the methods of the present invention as is effected by the systems of the present invention is independently characterized by spectral resolution ranging between 1 nm and 50 nm, e.g., 2-40 nm, 2-30 nm, 2-20 nm, or 2-10 nm, and spatial resolution ranging between 0.1 mm and 1.0 mm, preferably between 0.2 mm and 0.5 mm. Accordingly, the systems of the present invention are so designed and constructed so as to provide spectral resolution ranging between 1 nm and 50 nm and spatial resolution ranging between 0.1 mm and 1.0 mm. This combination of high spectral and spatial resolutions was, as of yet, never attempted for functional brain mapping and, the quality of results obtained using same, as is further exemplified in the Examples section that follows, is striking.

### Calculating OS and Blood Thickness (Volume) from Recorded Reflectance Spectra

A simple model that describes the reflection I(λ) of white light from the brain surface is obtained by assuming that the illumination signal is reflected from a partially absorbing and partially scattering tissue. The Consequently from the equations above one obtains that:

$$- \frac{ⅆ}{ⅆ λ} ln ( I / W ) ≅ 2 l ( \frac{ⅆ {ϵ}_{{HbO}_{2}}}{ⅆ λ} \overline{[ {HbO}_{2} ]} + \frac{ⅆ {ϵ}_{Hb}}{ⅆ λ} \overline{[ Hb ]} ) \qquad ( 17 )$$

Now, the oxygen saturation OS is measured in percent [%] and is defined as 100×[HbO₂]/([HbO₂]+[Hb]).

Defining c=[HbO₂]+[Hb]=const=8.98 μmole/mliter, which is the typical hemoglobin concentration in human blood, one obtains:

$$- \frac{ⅆ}{ⅆ λ} ln ( I / W ) = \frac{2 lc}{100} [ OS ( \frac{ⅆ {ϵ}_{{HbO}_{2}}}{ⅆ λ} - \frac{ⅆ {ϵ}_{Hb}}{ⅆ λ} ) + 100 \frac{ⅆ {ϵ}_{Hb}}{ⅆ λ} ] \qquad ( 18 )$$

This equation is the basic expression of the oxygen saturation model for a blood containing tissue according to the present invention. The left hand side contains only measured quantities (I,W), while the right hand side contains the known quantities from the literature ε_{HbO2}(λ) and ε_{Hb}(λ), and the unknowns l and OS.

One now has a set of n equations, where n is the number of wavelengths measured (typically about 100 data-points per spectrum), to solve with two unknowns: OS and l. Solving for OS we can now reconstruct the spectrum by applying the effective medium approximation:

μ(λ)=ε_{HbO2}(λ)·┌HbO₂┐+ε_{Hb}(λ)·[Hb] (19)

attenuation of the signal is described by the Beer-Lambert law, and the measured signal is thus given by:

I(λ)=W(λ)·exp[−a(λ)·2l] (15)

where W(λ) is the intensity of the incident light, I(λ) is the reflection of white light from the tissue, l is the penetration depth of the light beam, and a(λ) is the attenuation coefficient that consists of two contributions—the absorption coefficient μ(λ) and the scattering coefficient s(λ), i.e., a(λ)=s(λ)+μ(λ). In a blood containing tissue the attenuation characteristics are dominated by the absorption characteristics of hemoglobin, which varies non-monotonously with λ in the spectral range of 500 nm and 650 nm, while the scattering coefficient s(λ) is weakly dependent on the wavelength λ. Thus, for the wavelength range of 500 nm-650 nm it is assumed that da(λ)/dλ≅dη/dλ

approximately holds true. For μ(λ) in the case of hemoglobin, the effective medium approximation μ(λ)=ε_{HbO2}(λ)·┌HbO₂┐+ε_{Hb}(λ)·[Hb] (16) is applied, where [HbO₂] and [Hb] are the concentration of the oxygenated and deoxygenated hemoglobin, respectively, and ε_{HbO2}(λ) and ε_{Hb}(λ) are the oxygenated and deoxygenated hemoglobin absorption coefficients, respectively.

μ(λ) is now compared with the actual optical density of the spectrum to which, in pure theory, it should be identical. This comparison is named “fit” as how well the calculated spectrum actually fits the measured spectrum is determined.

Calculating l provides the blood thickness (indicative of volume).

### Generation of Oxygen Saturation and/or Blood Volume Difference Maps

Generation of oxygen saturation and/or blood volume color or intensity coded maps and color or intensity coded oxygen saturation and/or blood volume difference maps is addressed herein primarily in context of functional brain mapping, however, it will be appreciated that such maps can similarly be constructed for other tissues, including, but not limited to, the heart, liver, kidney, eye, etc., for example, monitoring the renewal of blood vessels in cases of skin flap implants, where this information is important for deciding on when to cut the flap, or for analyzing skin nevos for the purpose of performing an optical biopsy, reducing the need for recession of nevos, a process which at times is associated with complications, or for using in heart open surgery for the purpose of assessing the quality of blood supply, or for use with dye-involving processes, such as, but not limited to, use of ALA in PDT treatments or use of voltage sensitive dyes for monitoring brain activity, following, for example, a stimulus, oxygenation, deoxygenation, blood perfusion, etc.

In addition, while the present invention primarily addresses hemoglobin as a monitored substance, concentration and/or difference maps of any other substance, featuring spectral absorption properties in the visual or infrared range to which a spectral-imaging device is sensitive, can be similarly monitored.

Thus, according to a preferred embodiment of the present invention at least one threshold is used while generating the image highlighting differences among spectra of a tissue, such as the exposed cortex, as is further delineated below. Preferably the image highlighting differences among spectra of the tissue is highlighting oxygen saturation and/or blood volume differences. However, other substances may be monitored, including certain metabolites and other cellular components which are mentioned in the background section hereinabove.

Still preferably, at least one threshold is used while generating the image highlighting oxygen saturation and/or blood volume differences.

According to one embodiment, the threshold is effected by taking into account only picture elements in which an absolute oxygen saturation and/or blood volume is above a predetermined threshold, say above 30% of maximal value, above 40% of maximal value, above 50% of maximal value, above 60%, above 70%, above 80% or above 90% of maximal value, typically in the range of 70% -100% of maximal value. Different thresholds can be applied to data acquired prior to, during or following stimulation.

According to another embodiment, the threshold, alternatively or further includes taking into account only picture elements in which a difference in oxygen saturation and/or blood volume before and during or after the stimulation is above a predetermined second threshold, say about 1%, about 2%, about 3%, about 4%, about 5%, about 10% or about 20%.

According to another embodiment, the threshold, alternatively or further includes taking into account only picture elements for which the total intensity is above a predetermined threshold and painting black all other pixels. This serves as a means for eliminating picture elements that are included in the image but which are not part of the exposed cortex (pixels around the exposed cortex are typically of lower reflection energy and are thus eliminated).

According to still another preferred embodiment of the invention clusters of neighboring picture elements (above the first and the second thresholds) which include less than a predetermined number picture elements, say 1-5 picture elements, depending on the spatial resolution, are discarded.

Based on the data collected and the thresholds as described above the image highlighting differences among spectra of the exposed cortex collected before and during or after stimulation is constructed.

In addition, spectral images can be constructed based on the data collected before and/or during of after stimulation, highlighting absolute or relative values.

In any case the images can be color or intensity coded.

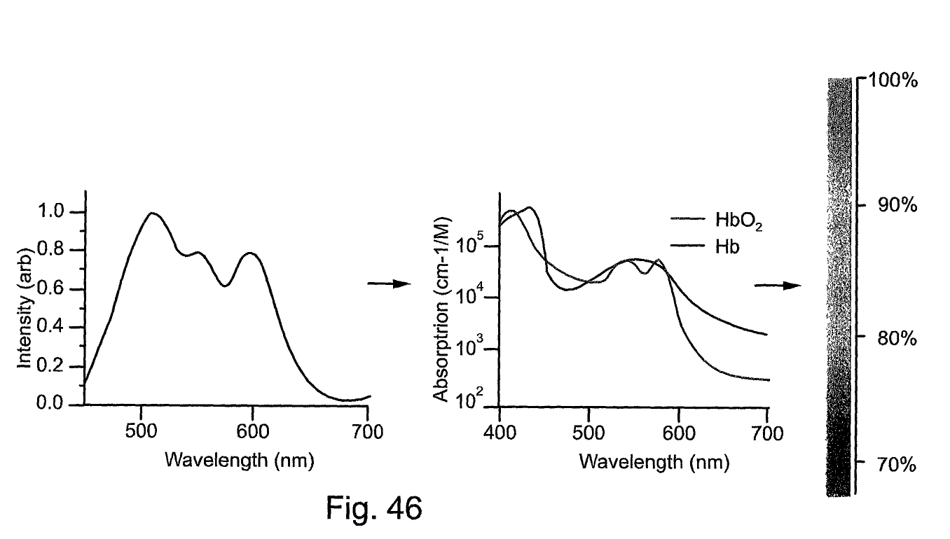

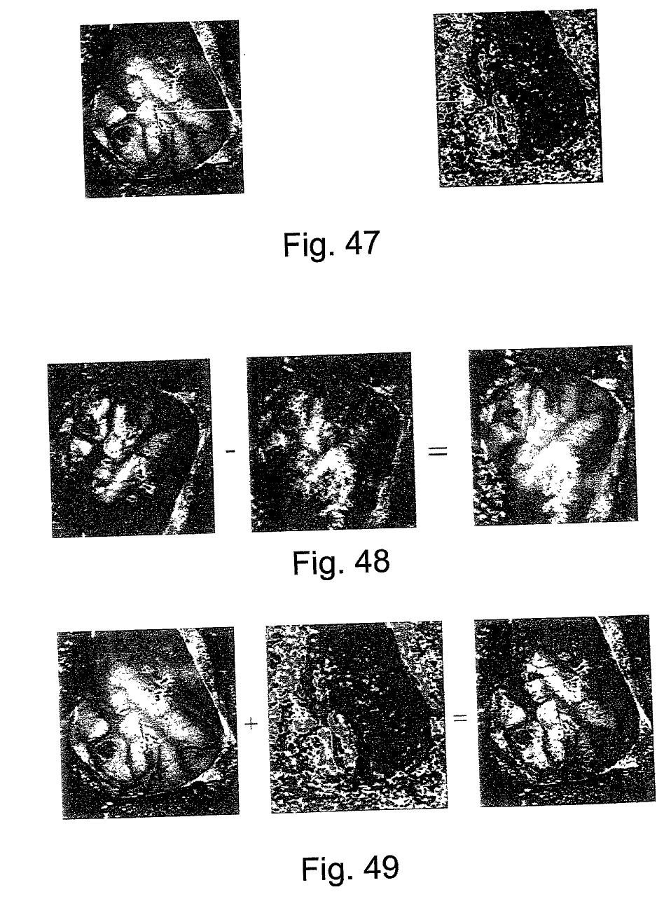

As shown in FIG. 46, according to the present invention, published hemoglobin absorption spectra are used to calculate the oxygen saturation value of each picture element, so as to create a color or intensity coded map. Thus, as shown in FIG. 47, each pixel is assigned a color according to its absolute or relative oxygen saturation value. As shown in FIG. 48, subtraction of an oxygen saturation map acquired pre stimulation from an oxygen saturation map acquired post stimulation, applying thresholds as described herein and overlaying the results on a grayscale image results in a comprehensive oxygen saturation difference map.

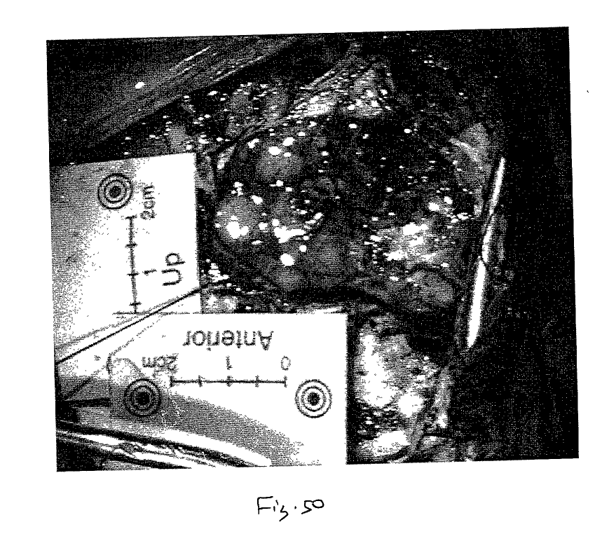

As shown in FIG. 49 a color or intensity coded map (oxygen saturation map in this case) can be overlaid on a grayscale image so as to obtain a composite image highlighting both anatomical features as well as functional features.

Thus, when highlighting differences, coding refers to the degree of difference, e.g., coded saturation and/or blood volume difference maps and is effected by one or more difference threshold. When highlighting absolute or relative values, coding refers to their absolute or relative levels, e.g., coded saturation and/or blood volume maps, and is effected by suitable one or more absolute or relative thresholds. In the latter case, the images are generated by attributing each of the pixels in the images a distinctive color or intensity according to, for example, oxygen saturation and/or blood volume characterizing its respective picture element in the cortex.

In a preferred embodiment of the invention coded images or maps are co-displayed either side by side with respect to, and/or overlaid (e.g., superimposed) over, an anatomical image of the examined tissue, e.g., the cortex.

The anatomical image, which is constructed from the spectral data collected before or during and/or after the stimulation can be an RGB image or a monochromatic (e.g., gray scale) image.

Thus, in a specific embodiment, the present invention provides a method of generating an oxygen saturation and/or blood volume difference map of a tissue of a subject. The method is effected by (a) illuminating the tissue of the subject with incident light; (b) at a first time point, acquiring a spectrum of each picture element of the tissue of the subject; (c) at a second time point, acquiring at least one additional spectrum of each picture element of the tissue of the subject; and (d) generating an image highlighting differences among spectra of the tissue acquired in steps (b) and (c), so as to generate the oxygen saturation and/or blood volume difference map of the tissue. Thresholds and other features as described above with respect to functional brain mapping are preferably applied in a similar manner.

According to this embodiment, the present invention also provides a system for monitoring oxygen saturation in a tissue. The system comprises a spectral imaging device and an image generating device, the spectral imaging device and the image generating device acting in synergy to produce an oxygen saturation difference map by highlighting tissue regions characterized by (a) having an absolute or relative level of oxygen saturation above a predetermined first threshold; (b) having an oxygen saturation difference above a predetermined second threshold; and/or (c) having a cluster size above a predetermined size.

Further according to this embodiment, the present invention also provides a system for monitoring blood volume in a tissue. The system comprises a spectral imaging device and an image generating device, the spectral imaging device and the image generating device acting in synergy to produce a blood volume difference map by highlighting tissue regions characterized by (a) having an absolute or relative level of blood volume above a predetermined first threshold; (b) having a blood volume difference above a predetermined second threshold; and (c) having a cluster size above a predetermined size.

FIG. 44 shows a color coded oxygen saturation map of a human cortex overlaid on a monochromatic anatomical image of the cortex.

FIG. 45 shows a color coded blood volume map of a human cortex overlaid on a monochromatic anatomical image of the cortex.

A pair of such maps is used according to the present invention to calculate and display color or intensity coded oxygen saturation and/or blood volume difference maps.

According to a presently preferred embodiment of the invention, a plurality of images highlighting differences among spectra are displayed either superimposed, overlaid or integrated into a cumulative difference image or map, as is further described and exemplified in the Examples section that follows.

### Spectral Data Acquisition Time Considerations

Different total acquisition times can be considered when monitoring different biological process. The scheme used by the spectral imaging related publications cited in the background section employed measurements effected about two seconds post stimulation of the brain. Measurements occurring within this time period detect the very early and highly dynamic changes of blood-flow which is coupled to neural activation. However, obtaining high-quality maps within about two seconds is an impossible task due to the beating of the brain and the low signal-to-noise ratio of the acquired images.

The following provides considerations for measurements executed at longer time periods, say 10-15 seconds, post stimulation, which were used while reducing the present invention to practice, yielding unexpected results.

Ten-15 seconds post stimulation the hemodynamic changes are not as dramatic as shortly after the stimulation and are probably more diffuse. However, the advantage of measuring in this delayed interval is that one is able to construct high-quality oxygen saturation maps that overcome the problem of brain beating by averaging over a large (e.g., >10) number of beat cycles and obtaining high signal-to-noise ratio images by collecting a large number of photons (and so reduce the effect of “shot noise”, the major noise contributor in this kind of setup). The measurements performed by the inventors of the present invention prove that the oxygen saturation changes, resulting from neuronal activation, are still evident during this time span.

The upper limit for measurement is probably 20-30 seconds post stimulation because (i) in the operation room, when operating awake patients, a task should not exceed 10-20 seconds; and (ii) post 20-30 seconds one risks measuring other, not-anticipated, stimuli of the brain.

Thus, according to a preferred embodiment of the present invention spectral data collection is performed during at least N brain beats of the subject, wherein N is an integer selected from the group consisting of two, three, four, five, six, seven, eight, nine, ten and an integer between and including eleven and forty. Preferably, the step of spectral data collection post stimulation is effected more than about 3-5 seconds and preferably between about 3-5 and about 30 seconds following initiation of stimulation. According to a preferred embodiment the stimulation prolongs about 3-5 to about 30 seconds, preferably about 10 to about 20 seconds. Preferably, stimulation prolongs throughout the entire second measurement period.

According to a preferred embodiment the reflectance spectrum for each picture element and for each spectral data collection step is an averaged reference spectrum of N measurements (for filter based system) or N brain-beats (for interferometer based system), wherein N is an integer and equals at least 2 and is preferably between 5 and 20, say about 10.

Thus, the goal of the measurement is to provide high-signal-to-noise ratio images within 10-15 seconds of stimulation overcoming the beating problem of the brain. The following translates these considerations to the use of a filters-based spectral imaging device. The following terminology shall apply.

An image is one exposure of the CCD through one filter at an exposure time that brings the recorded signal close to the CCD full well.

A set is sequential acquisition of images through all the filters used in the system.

A layer is a composition of all images, from the different sets, of a certain filter.

Using the above terminology a recommended acquisition scheme is described below:

For overcoming the beating problem of the exposed cortex at least 10 sets should be acquired at a rate that is not correlated with the beating. The time for acquiring a single set should be 10-20 seconds, say about 15 seconds.

When constructing the layers, spatial registration algorithms should be used to fix possible shifts between images. Such algorithms are well known in the art and are therefore not further described herein.

Once layers are constructed, a file is created where each picture element is given a discrete spectrum composed of its intensity value in each one of the layers.

The points spectrum of each pixel is then interpolated and the interpolation is used as the basis for calculating the oxygen saturation or blood volume value of the picture element represented by the pixel according to the method described herein, or using any other saturation or volume calculation method. Interpolating the discrete spectrum of each pixel is not a necessity, as the calculations can be performed directly on the discrete data (see FIGS. 12 and 13).

PCT Application US97/08153, which is incorporated herein by reference, teaches a method for spatial registration and spectral correction for interferometer-based spectral imaging devices which can be used to obtain spectral images of a moving object. The method is effected by (a) using an interferometer-based spectral imaging device for acquiring spatial and spectral information of the moving object; and (b) correcting the spatial and spectral information for movements of the moving object via a spatial registration and spectral correction procedures for obtaining corrected spatial and spectral information. The teachings of this PCT application can be integrated with the present invention so as to enable spectral imaging of a beating cortex during shorter acquisition times.

### Stimulation Protocols and Additional Considerations Related to Brain Stimulation

According to a preferred embodiment of the present invention and as is in many cases of neurosurgeries practiced anyway the subject is awake during the procedure. Alternatively, the subject is anesthetized. According to one embodiment of the present invention stimulation is effected by asking the (awake) subject to perform a task, such as, but not limited to, reading, speaking, listening, viewing, memorizing, thinking and executing a voluntary action (e.g., moving a limb, blinking one or both eyes, etc.). According to another embodiment, stimulation is effected by passively stimulating the brain of the (awake or anesthetized) subject (e.g., inducing brain activity) through the peripheral nervous system by, for example, directing light into the eyes, voice into the ears or by electrical stimulation of the skin at different body locations. Direct electrical stimulation of the brain using electrodes is also applicable.

Typically, during a neurosurgery, medical lines, e.g., infusion, ECG leads, etc., are connected to the subject. Preferably, the medical lines are connected to the subject on a single side thereof. Preferably, the medical lines are connected to the subject at locations which are less communicating with the exposed portion of the cortex of the subject. Thus, if the right hemisphere of the cortex is exposed, the medical lines should be connected to the left side of the subject, whereas, if the left hemisphere of the cortex is exposed, the medical lines should be connected to the right side of the subject.

Occasionally, it may be advantageous to acquire a reflectance spectrum of each picture element of at least the portion of the exposed cortex of the subject when the patient is briefly (for a short time) anesthetized. This data can locate active brain regions and may serve as reference when interpreting the results of images generated when the patient is awake. Briefly anesthetizing the patient can be effected by, for example, propofol, allowing for a 5-10 minute regain of consciousness once administration is stopped.

Other manipulations that can be used during open skull surgery include the use of (i) identical paradigms while mapping according to the present invention as when mapping with pre-operational fMRI for the purpose of comparing these two data sets; (ii) medicine for the purpose of reducing the OS of the cortex, so that a task can be repeated, for the purpose of testing for repetition as a means of confirming results; (iii) measures for reducing the sensory input to the patient such as providing the patient with earplugs, eye covers, local anesthesia, etc.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### Illumination Considerations

It is readily appreciated that the use of illumination presently existing in a brain surgery operating room (OR) for illuminating the cortex for the purpose of acquiring spectral-images is desired and is advantageous over using dedicated illumination (direct current, DC, illumination type) as described hereinabove. Operating room illumination is typically of an alternating current (AC) type and is therefore characterized by a frequency and a frequency time corresponding to the electrical network of a geographical region (typically about 50 (frequency time of 20 milliseconds) or about 60 Hz (frequency time of 16.66 milliseconds), depending on the region). Using a filter-based system for acquiring spectral-images reduces the need of a stable DC illumination source, provided that the following two limitations are met:

1. The exposure times used should be the same for all filters, i.e., no single filter uses an exposure time shorter or longer then any other filter in the filter-wheel.

2. The exposure times used are multiplications of the electrical frequency. For example in a network with a frequency of 50 Hz each time cycle should be about 20 milliseconds. The allowed exposure intervals are therefore multiplicities of about 20 milliseconds, e.g., say about 40 milliseconds, about 60 milliseconds, etc. In a 60 Hz network the cycle time is 16.66 milliseconds. This can be rounded to 17 milliseconds and allow for multiplicities of about 17 milliseconds, e.g., about 33 milliseconds, about 50 milliseconds, etc. Failing to take the frequency into consideration, individual filters will sample different phases of the illumination cycle and will therefore show different intensity values, which are actually, an artifact.

Thus, according to an embodiment the present invention the spectral data is collected via a filters-based spectral imaging device and the illumination is effected by an illumination device operated with an alternating current characterized by a frequency time wherein (i) an exposure time of all filters of the filters-based spectral imaging device is substantially equal; and (ii) an exposure time of each of the filters is a multiplicity of the frequency time by an integer.

### Image Orientation

According to the present invention a “label” which is placed on the skull, near the exposed cortex portion is used for providing three functions as follows:

1. The label contains textual or symbol information pertaining to the anatomical orientation of the craniotomy and appears in the resulted images, so at to simplify the process of orienting an image with respect to the actual craniotomy, as the imaging device might cause a rotation of the image.

2. The label supplies a “white target” reference spectrum for calculating oxygen saturation as described herein. A “white target” is a target that reflects incident light without altering the spectrum of the light. In other words, the “white target” has a flat (constant) absorption coefficient in a spectral range of interest (say, between the c400-700 nm spectral range). The refraction index of the “white target” can be high or low, resulting in black, gray or white color in the image.

3. The label, either by its size which is known to the user or by the addition thereto of scale marks, also serves as a scale.

FIG. 50 shows two labels (marked as “Up” and “Anterior”, respectively) placed around a craniotomy marking the “Up” direction and the “Anterior” direction of the craniotomy (thereby providing a “North”-“South” type orientation). These labels are placed by the neurosurgeon at the beginning of the imaging session and greatly simplify the task of understanding the orientation of the image presented on the monitor.

The system's software is designed to automatically find the “white target” area within an acquired image and to extract an average spectrum from it, which average spectrum serves as the W(λ) (see Equation 15) mentioned hereinabove with respect to the oxygen saturation calculation. Having W(λ) so sampled eliminates the necessity to acquire a separate white target spectral-image at the beginning of the imaging session. It further eliminates the need of using an illumination source with known spectral characteristics and allows for using the illumination already present in the operating room without introducing special dedicated illumination for illuminating the cortex.

When implementing this embodiment of the present invention, i.e., co-acquiring the white target within the image, it is important to have the refraction index of the white target close to (say ±10% or ±20%) the refraction index of the cortex. If the white target is brighter then the cortex it will become saturated at a light level such that the cortex will not be illuminated sufficiently.

### EXAMPLES

Reference is now made to the following examples, which together with the above descriptions, illustrate the invention in a non limiting fashion. Spectral data presented herein was acquired using SPECTRACUBE 300 spectral imaging device manufactured and distributed by Applied Spectral Imaging Ltd., Migdal Haeemek, Israel. The procedures described herein were approved by a Helsinki Committee. After they were explained of the procedures and their experimental nature, all patients reported herein signed an informed consent prior to operation.

### Example 1

### fMRI vs. Exposed Cortex Images Obtained via Spectral Imaging

This example demonstrates the difference between preoperational images (be it CT, PET or fMRI) and the way the exposed cortex appears to the operating neurosurgeon during operation.

FIG. 14 shows a T1-weighted image acquired to localize anatomy within which evoked function will be imaged. The brain is segmented to create a binary mask for application to the fMRI image. FIG. 15 shows an fMRI image acquired during photic stimulation. FIG. 16 shows the masking of FIG. 15 with the T1 brain mask segments activity localized to the brain. As shown in FIG. 16, selection of a given threshold reveals areas of evoked response function. These fMRI images were taken from the web site of the Mayo clinic (USA), (http://www.mayo.edu/) and present typical fMRI results.

FIG. 10 shows a color (RGB) image reconstituted from spectral data acquired on awake patient undergoing neurosurgery. Comparing the fMRI images of FIGS. 14-16 to the color image of FIG. 10, which is identical to the view seen by the operating surgeon, reveals that interpreting brain anatomy from the fMRI image, is not a trivial task.

Furthermore, the anatomy of the brain changes to a great extent post craniotomy due to the inner-cortical pressure, which changes are not at all addressed by preoperational images.

### Example 2

### Calculating Oxygen Saturation Difference Maps by Applying Various Thresholds

The images shown herein were derived from a 58 year-old female, diagnosed for a right parietal enhancing tumor (GBM), which underwent tumor resection under general anesthesia.

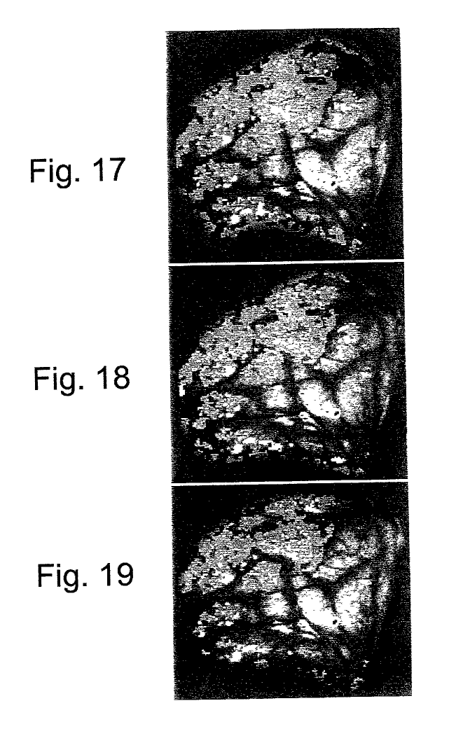

The images shown in FIGS. 17-27 are difference maps created by comparing a base image with an image acquired post left palm electrical stimulation and demonstrate the importance of using thresholds when highlighting oxygen saturation differences in accordance with the teachings of the present invention. Overall oxygen saturation values in this patient are low and represent a typical values of a patient under general anesthesia. The patient was respirated and monitored with the following physiological parameters:

Respiration Rate—10 per minute

Total Volume—0.7 liter

Blood Pressure—125/60

End Tidal CO₂—30 mmHg

Medication: Remphentanil 0.18 μl/kg/minute; Propofol 30 mg/hour.

FIG. 17 shows an oxygen saturation difference map overlaid on a monochromatic gray-scale image of the cortex highlighting pixels (in red) corresponding to brain regions (picture elements) that underwent an increase in oxygen saturation (OS) that reached an OS level greater then 40% and have risen by more then 1% post left palm electrical stimulation.

FIG. 18 shows an oxygen saturation difference map overlaid on a monochromatic gray-scale image of the cortex highlighting pixels (in red) corresponding to brain regions (picture elements) that underwent an increase in oxygen saturation (OS) that reached an OS level greater then 45% and have risen by more then 1% post left palm electrical stimulation.

FIG. 19 shows an oxygen saturation difference map overlaid on a monochromatic gray-scale image of the cortex highlighting pixels (in red) corresponding to brain regions (picture elements) that underwent an increase in oxygen saturation (OS) that reached an OS level greater then 50% and have risen by more then 1% post left palm electrical stimulation.

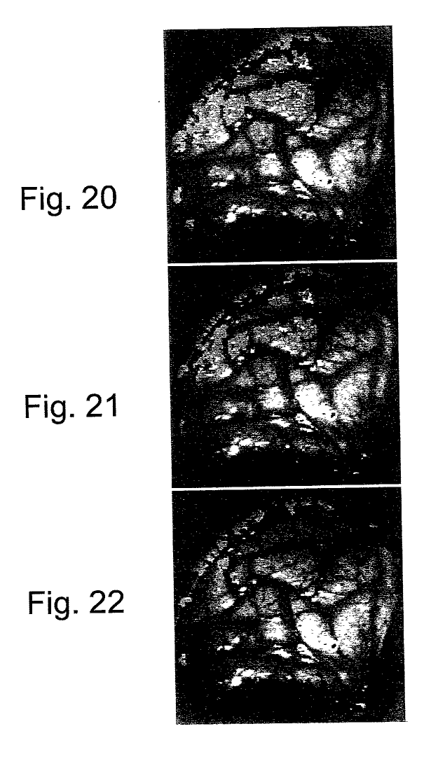

FIG. 20 shows an oxygen saturation difference map overlaid on a monochromatic gray-scale image of the cortex highlighting pixels (in red) corresponding to brain regions (picture elements) that underwent an increase in oxygen saturation (OS) that reached an OS level greater then 55% and have risen by more then 1% post left palm electrical stimulation.

FIG. 21 shows an oxygen saturation difference map overlaid on a monochromatic gray-scale image of the cortex highlighting pixels (in red) corresponding to brain regions (picture elements) that underwent an increase in oxygen saturation (OS) that reached an OS level greater then 60% and have risen by more then 1% post left palm electrical stimulation.

FIG. 22 shows an oxygen saturation difference map overlaid on a monochromatic gray-scale image of the cortex highlighting pixels (in red) corresponding to brain regions (picture elements) that underwent an increase in oxygen saturation (OS) that reached an OS level greater then 65% and have risen by more then 1% post left palm electrical stimulation.

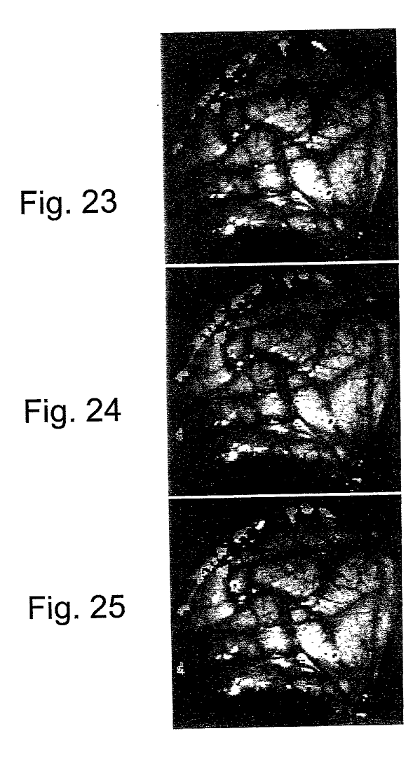

FIG. 23 shows an oxygen saturation difference map overlaid on a monochromatic gray-scale image of the cortex highlighting pixels (in red) corresponding to brain regions (picture elements) that underwent an increase in oxygen saturation (OS) that reached an OS level greater then 70% and have risen by more then 1% post left palm electrical stimulation.

FIG. 24 shows an oxygen saturation difference map overlaid on a monochromatic gray-scale image of the cortex highlighting pixels corresponding to brain regions (picture elements) that underwent an increase in oxygen saturation (OS) that reached an OS level greater then 65% and have risen by more then 1% (red) or less than 1% (yellow) post left palm electrical stimulation.

FIG. 25 shows an oxygen saturation difference map overlaid on a monochromatic gray-scale image of the cortex highlighting pixels corresponding to brain regions (picture elements) that underwent an increase in oxygen saturation (OS) that reached an OS level greater then 65% and have risen by more then 3% (red) or less than 3% (yellow) post left palm electrical stimulation.

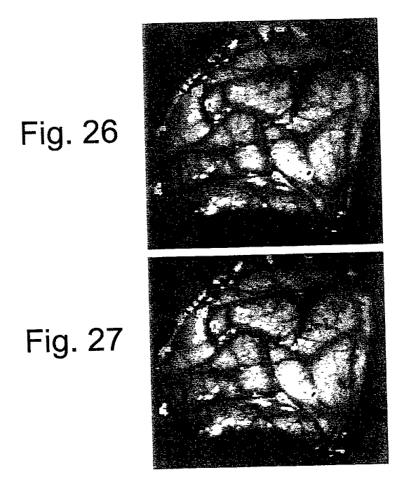

FIG. 26 shows an oxygen saturation difference map overlaid on a monochromatic gray-scale image of the cortex highlighting pixels corresponding to brain regions (picture elements) that underwent an increase in oxygen saturation (OS) that reached an OS level greater then 65% and have risen by more then 5% (red) or less than 5% (yellow) post left palm electrical stimulation.

FIG. 27 shows an oxygen saturation difference map overlaid on a monochromatic gray-scale image of the cortex highlighting pixels corresponding to brain regions (picture elements) that underwent an increase in oxygen saturation (OS) that reached an OS level greater then 65% and have risen by more then 10% (red) or less than 10% (yellow) post left palm electrical stimulation.

These Figures demonstrate the importance of using two thresholds, the first relates to the relative (or absolute) value of oxygen saturation, whereas the second relates to the change thereof post stimulation.

It will be appreciated that a similar analysis can be made with respect to blood volume, which takes into account the sum value of oxy and deoxy-hemoglobin.

### Example 3

### Wernike's Area Mapping During Awake Craniotomy

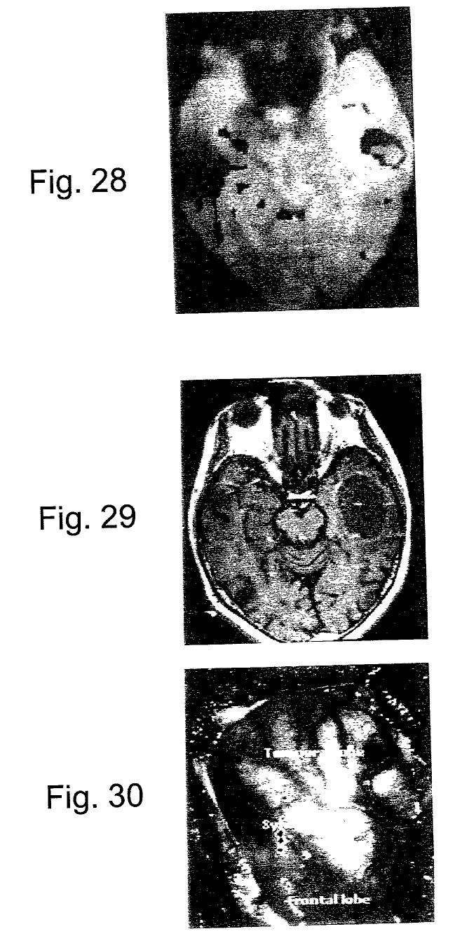

An 80 years old male diagnosed with lung cancer 12 years prior to admission for a left temporal cystic lesion (found to be a metastasis). The patient suffers from cognitive dysfunction (anterograde amnesia) and dysphasia. fMRI imaging showed Wernike's Area to be located adjacent to the tumor on the Superior Temporal Gyros (STG), see FIGS. 28-30.

FIG. 28 shows an fMRI image demonstrating the activation of Wernike's area (the orange spot on the right. FIG. 29 is a CT image showing a section of the brain, the tumor is clearly seen on the right-hand side (actually the left hemisphere of the brain). FIG. 30 is a gray-scale orientation image as observed by the spectral imaging device employed.

The patient underwent awake craniotomy for tumor resection. Physical parameters during craniotomy:

OS 100% —measured using pulse oxymeter on toe.

PCO₂—38

BP 80 systole

Medication at this stage:

Propofol 70 mg/minute

Remiphentanil 01 μl/kg/minute

Post craniotomy the Propofol is stopped and the BP rises to 100 systole. The patient is now awake and asked to start performing different tasks.

At 17:14 the patient performs a counting task in his native language (Polish), a task in which he succeeds. At 17:15 he is asked to translate words from Hebrew to Polish, a task in which he succeeds.

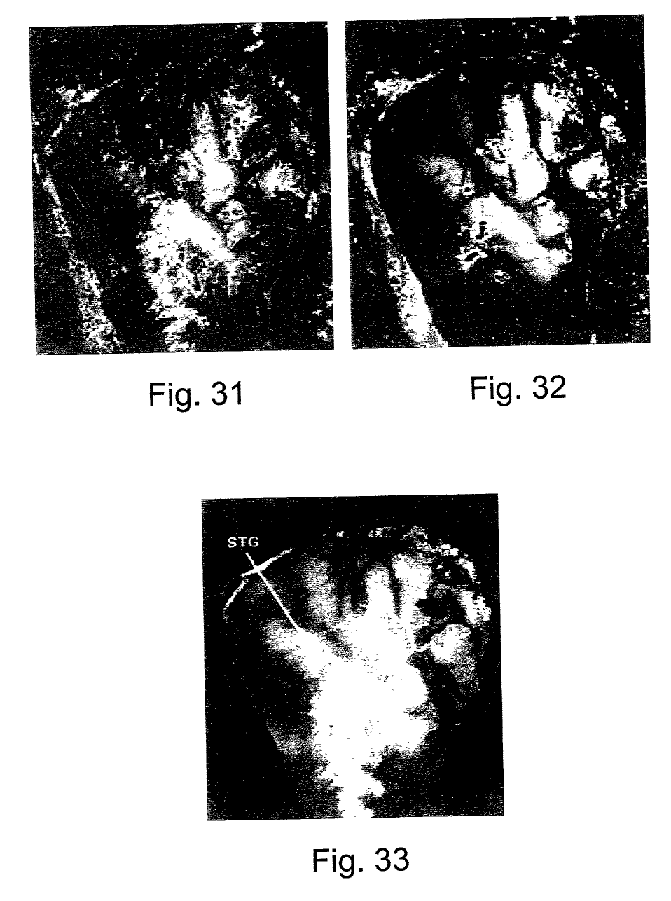

FIGS. 31 and 32 show color coded oxygen saturation maps of the patient's cortex pre and post translation task. The data represented by these images was used for locating Wernike's area (see FIG. 33), which is only activated by the more cognitively complex task of translation.

### Example 4

### Motor Cortex and Associated Speech Areas During Awake Craniotomy

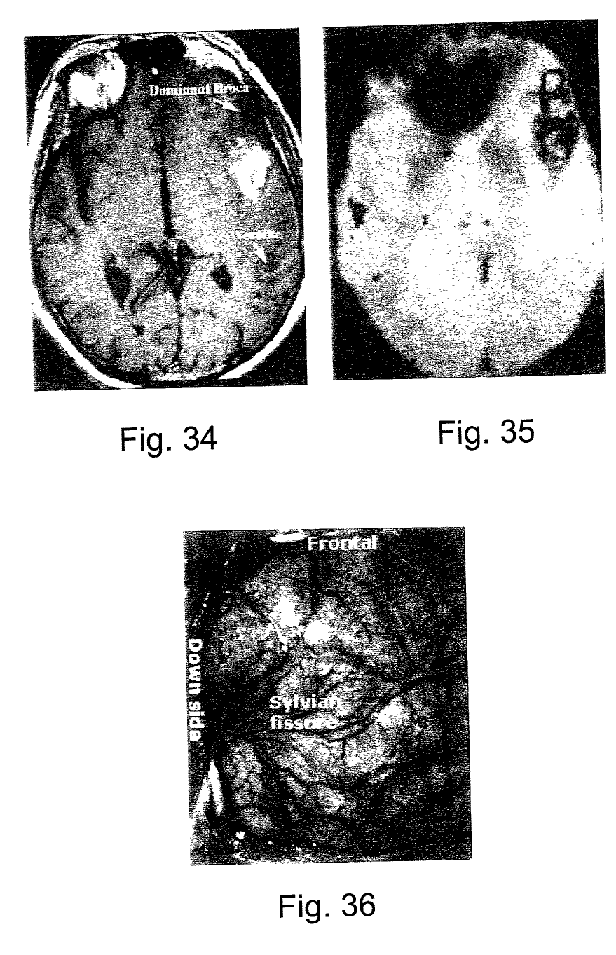

A 50-year-old male diagnosed one year ago with melanoma. Recent headaches led to diagnosis of metastasis. CT showed a single tumor strand in left temporal area (see FIG. 34). fMRI showed dominant Broca (see FIG. 35). The patient underwent awake craniotomy for tumor resection.

At 11:18 an acquisition is performed while the patient is naming different objects (pen, cigarettes, etc.).

At 11:21 an acquisition is performed while the patient is asked to repeat sentences, which he hears. This is called a repetition task. The patient had a hard time repeating the sentences.

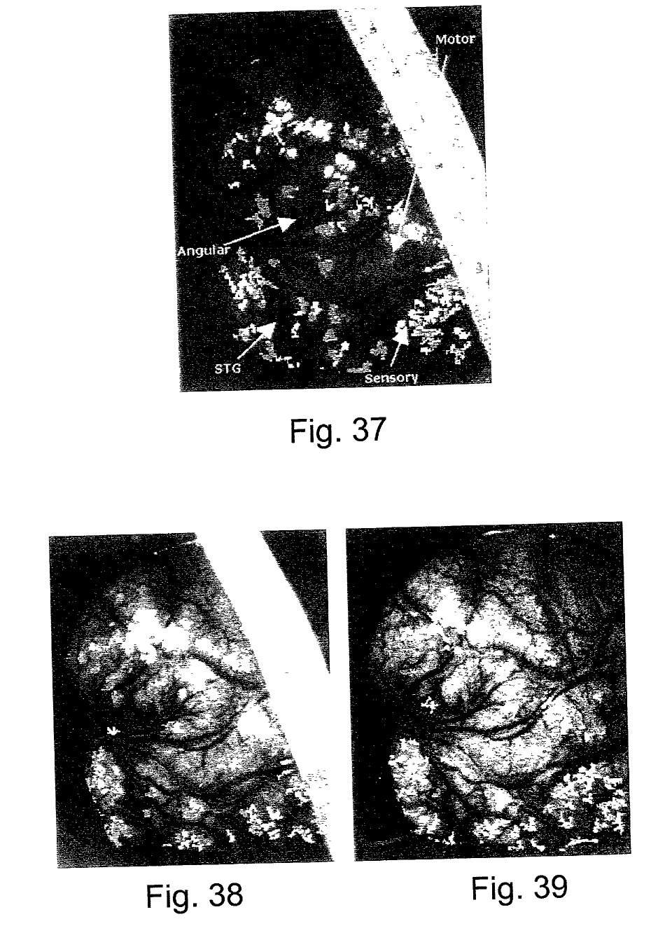

In any case, FIG. 37 shows an oxygen saturation difference map highlighting speech-associated areas.

Later attempts to locate dominant speech area (Broca) using direct cortical stimulation (via electrode placed in contact with the motor cortex), while the patient was performing a speech task, failed to cause any speech disturbances. This leads to the conclusion that the Broca itself was not included in the craniotomy.

At 11:23 an acquisition is performed while the patient is touching fingers of his right hand (he performed well).

At 11:25 an acquisition is performed during a mouth movement task (the patient was told to open and close his mouth and performed well).

These two later acquisitions were used for creating oxygen saturation difference maps highlighting changes in the motor cortex (FIGS. 38-39) as a result of touching fingers and open and close mouth tasks.

### Example 5

### Associated Visual Cortex Mapping During General Anesthesia

A 72 years-old female. Progressive dysphasia and headaches led to diagnosis of a large left parieto-occipital enhancing tumor (GBM). Underwent left occipital craniotomy under general anesthesia. Optokinetic stimulation to an open left eye was performed after dural opening.

Physical parameters during craniotomy:

Patient placed in “park bench” position.

100% OS (pulse oxymeter)

PO₂ 450

PCO₂ 35

Medications:

Remphentanil 0.1 μl/kg/minute

Propofol 100 mg/hour

Aontopintunine 12 μl/kg/minute

At 11:03 a base image was acquired post craniotomy. The lid of the left eye was opened using a separating tool. The pupil was visible and small.

At 11:10 data acquisition was performed while an illuminated Opto-kinetic strip was passed before the patient's left eye.

FIG. 42 is a gray-scale orientation image as observed by the spectral imaging device employed.

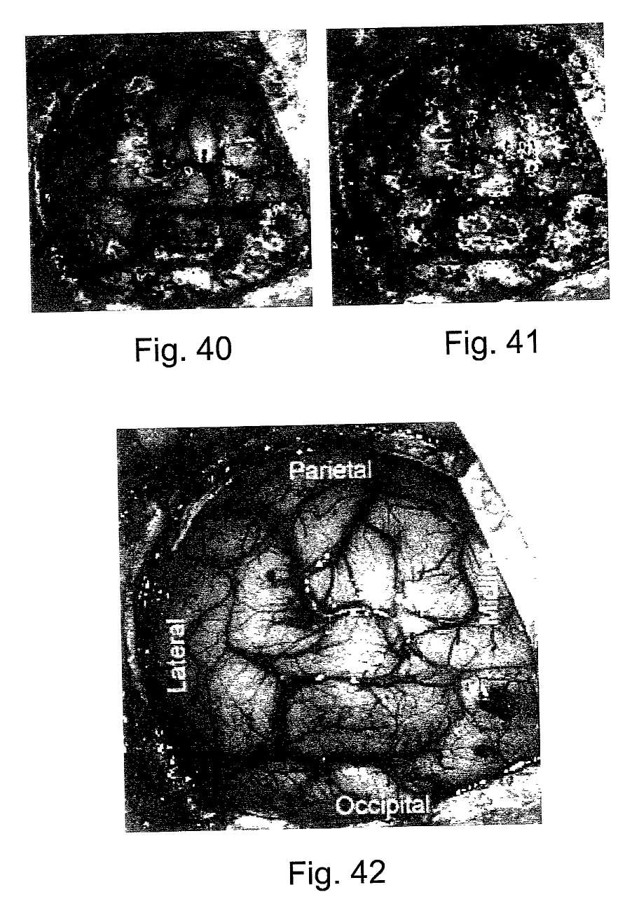

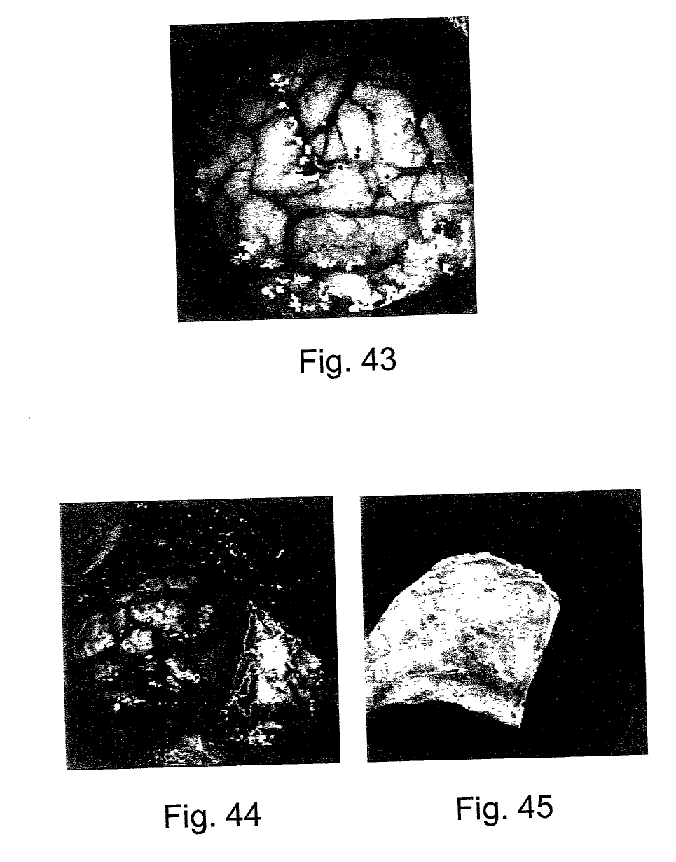

FIGS. 40 and 41 show color coded oxygen saturation maps of the patient's cortex pre and post passive optical left eye stimulation. The data represented by these images was used for locating visual associated cortex regions (see FIG. 43). It is assumed that the primary visual cortex is not visible in the craniotomy. The larger red area in the lower portion of the map highlights an area affected by the optical stimulation. The anatomy implies, however, that this area is not the primary visual cortex, rather an associated area.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

### Example 6

### Cumulative Difference Display

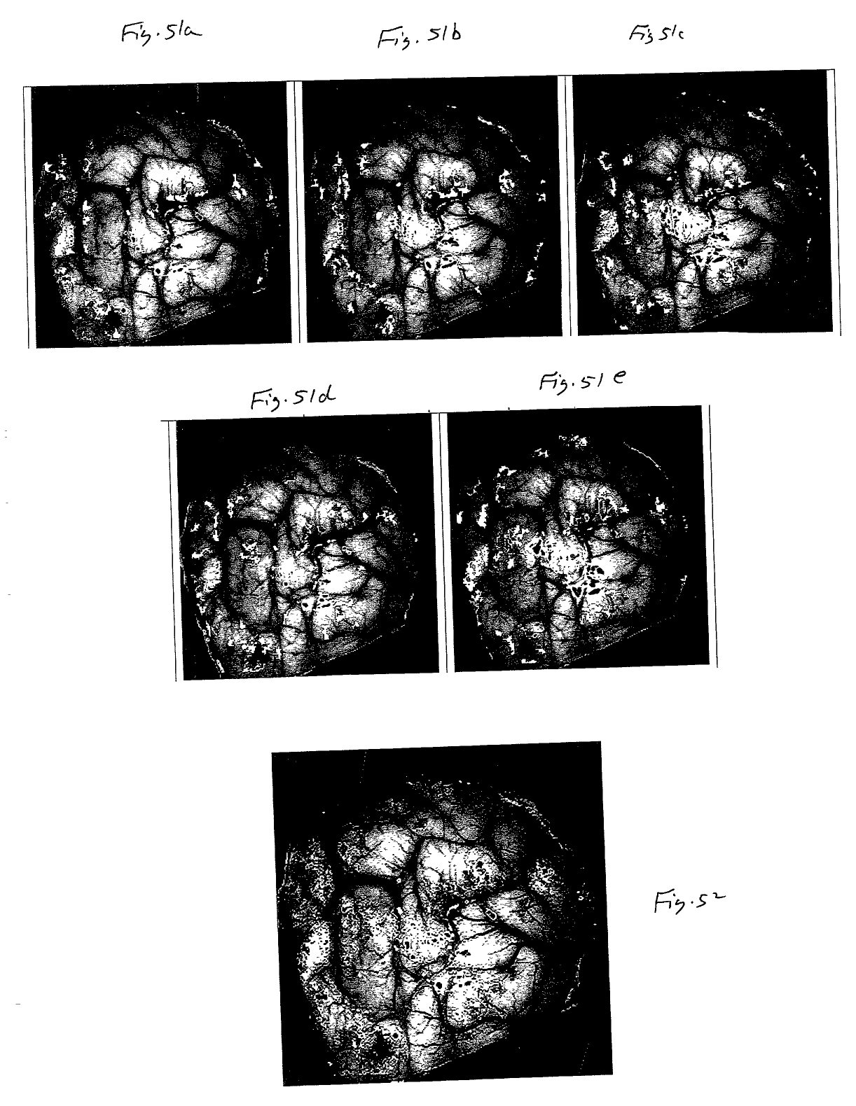

FIGS. 51a-e show a set of difference images calculated during several imaging sessions, where visual stimulation was performed on a patient under full anesthesia conditions by flashing light into an eye and alternatively by passing objects in front of the eye.

FIG. 52 shows a cumulative differences display according to the present invention, which integrates, overlays or superimposes the data of FIGS. 51a-e, to thereby better define brain regions which are active following visual stimulation.

FIGS. 51a-e and as a result also FIG. 52 show the spatial arrangement of increase in oxygen saturation. Decreased oxygen saturation is not presented, yet it can be displayed in a similar fashion. In addition, both regions characterized by increase and decrease of oxygen saturation can be co-displayed in an overlaid fashion as in FIG. 52.

The need for cumulative display arises because during higher-level stimulation protocols (such as those used for detecting speech associated functional cortical areas), the activated brain regions are typically not very well spatially defined, rather different regions, within the exposed imaged cortex are highlighted in different sessions or when employing different stimulation. It is therefore in many cases insufficient for the neurosurgeon to view any one particular difference image (as in FIGS. 51a-e) in order to distinctively identify the active cortical areas associated with a function. The cumulative differences display described herein overcomes this problem, by presenting all of the information in a single, superior image.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A method of functional brain mapping of a subject comprising the steps of:
(a) illuminating an exposed cortex of a brain or portion thereof of the subject with incident light;
(b) acquiring a reflectance spectrum of each picture element of at least a portion of the exposed cortex of the subject;
(c) stimulating the brain of the subject;
(d) during or after step (c) acquiring at least one additional reflectance spectrum of each picture element of at least the portion of the exposed cortex of the subject; and
(e) generating an image highlighting differences among spectra of the exposed cortex acquired in steps (b) and (d), so as to highlight functional brain regions.

2. The method of claim 1, further comprising the step of using at least one filter to adjust the spectrum of the incident light.

3. The method of claim 1, wherein each of steps (b) and (d) is independently characterized by spectral resolution ranging between 1 nm and 50 nm and spatial resolution ranging between 0.1 mm and 1.0 mm.

4. The method of claim 1, wherein each of steps (b) and (d) is effected via an interferometer-based spectral imaging device.

5. The method of claim 1, wherein each of steps (b) and (d) is effected via a filters-based spectral imaging device.

6. The method of claim 1, further comprising the steps of generating individual spectra-images from spectra acquired in steps (b) and (d).

7. The method of claim 6, wherein said spectral-images are generated by attributing each of the pixels in the images a distinctive color or intensity according to oxygen saturation and/or blood volume characterizing its respective picture element in the cortex.

8. The method of claim 1, wherein the subject is awake.

9. The method of claim 1, wherein the subject is anesthetized.

10. The method of claim 1, wherein step (c) is effected by asking the subject to perform a task.

11. The method of claim 10, wherein said task is selected from the group consisting of reading, speaking, listening, viewing, memorizing, thinking and executing a voluntary action.

12. The method of claim 1, wherein step (c) is effected by a method selected from the group consisting of passively stimulating the brain through the peripheral nervous system of the subject and directly stimulating the cortex.

13. The method of claim 1, further comprising the step of generating an anatomical image of the exposed cortex and co-displaying said image highlighting differences among spectra of the exposed cortex and the anatomical image of the exposed cortex.

14. The method of claim 13, wherein said image highlighting differences among spectra of the exposed cortex and the anatomical image of the exposed cortex are co-displayed side by side.

15. The method of claim 13, wherein said image highlighting differences among spectra of the exposed cortex and the anatomical image of the exposed cortex are superimposed.

16. The method of claim 1, wherein step (e) comprises a use of at least one threshold while generating the image highlighting differences among spectra of the exposed cortex acquired in steps (b) and (d).

17. The method of claim 1, wherein said image highlighting differences among spectra of the exposed cortex acquired in steps (b) and (d) is color or intensity coded.

18. The method of claim 1, wherein medical lines are connected to the subject on a single side thereof.

19. The method of claim 1, wherein medical lines are connected to the subject on a right or left side thereof.

20. The method of claim 1, wherein medical lines are connected to the subject at locations which are less communicating with the exposed portion of the cortex of the subject.

21. The method of claim 7, wherein said step (e) is characterized by highlighting oxygen saturation and/or blood volume differences of about at least 10%.

22. The method of claim 7, wherein said step (e) is characterized by highlighting oxygen saturation differences and/or blood volume of about at least 5%.

23. The method of claim 8, further comprising the step of also acquiring a reflectance spectrum of each picture element of at least the portion of the exposed cortex of the subject when the patient is briefly anesthetized.

24. The method of claim 1, wherein each of steps (b) and (d) is performed during at least N brain beats of the subject, wherein N is an integer selected from the group consisting of two, three, four, five, six, seven, eight, nine, ten and an integer between and including eleven and forty.

25. The method of claim 11, wherein step (d) is executed more than about 3-5 seconds after initiation of step (c).

26. The method of claim 1, wherein step (d) is executed between about 5 and about 30 seconds after initiation of step (c).

27. The method of claim 1, wherein said stimulation prolongs about 5 to about 30 seconds.

28. The method of claim 1, wherein said stimulation prolongs about 10 to about 20 seconds.

29. The method of claim 5, wherein said filters-based spectral imaging device includes filters selected so as to collect spectral data of intensity peaks or steeps characterizing one or more spectrally monitored substances.

30. The method of claim 5, wherein said filters-based spectral imaging device includes filters selected so as to collect spectral data of intensity peaks or steeps characterizing hemoglobin selected from the group consisting of deoxy-hemoglobin, oxy-hemoglobin and deoxy-hemoglobin and oxy-hemoglobin.

31. The method of claim 30, wherein each of said filters is individually about 5 to about 15 nm full-width-at-half-maximum filter.

32. The method of claim 30, wherein each of said filters is individually about 10 nm full-width-at-half-max filter.

33. The method of claim 30, wherein said filters include N filters selected from the group consisting of an about 540 nm maximal transmittance filter, an about 575 nm maximal transmittance filter, an about 555 nm maximal transmittance filter, an about 513 nm maximal transmittance filter and an about 600 nm maximal transmittance filter, whereas N is an integer selected from the group consisting two, three, four and five.

34. The method of claim 33, wherein N equals two.

35. The method of claim 33, wherein N equals three.

36. The method of claim 33, wherein N equals four.

37. The method of claim 33, wherein N equals five.

38. The method of claim 30, wherein said filters include at least one multiple chroic filter.

39. The method of claim 30, wherein said filters include at least one filter of maximal transmittance at a wavelength which corresponds to at least one isosbasthic point of deoxy-hemoglobin and oxy-hemoglobin and at least one additional filter of maximal transmittance at a wavelength which corresponds to at least one non-isosbasthic point of deoxy-hemoglobin and oxy-hemoglobin.

40. The method of claim 1, wherein said reflectance spectrum of step (b) is an averaged reference spectrum of N measurements, wherein N is an integer and equals at least 2.

41. The method of claim 1, wherein said reflectance spectrum of step (d) is an averaged reference spectrum, wherein N is an integer and equals at least 2.

42. The method of claim 1, further comprising the steps of spatially registrating spectral data acquired in steps (b) and (d).

43. The method of claim 1, wherein said image highlighting differences among spectra of the exposed cortex acquired in steps (b) and (d) is highlighting oxygen saturation and/or blood volume differences.

44. The method of claim 43, wherein step (e) comprises a use of at least one threshold while generating the image highlighting differences among spectra of the exposed cortex acquired in steps (b) and (d) of oxygen saturation and/or blood volume differences.

45. The method of claim 44, wherein said at least one threshold includes taking into account only picture elements in which, in step (b), in step (d) or both, an absolute oxygen saturation and/or blood volume is above a predetermined first threshold.

46. The method of claim 45, wherein said at least one threshold further includes taking into account only picture elements in which a difference in oxygen saturation and/or blood volume is above a predetermined second threshold.

47. The method of claim 46, wherein clusters of neighboring picture elements above said first and said second threshold, said clusters include less than a predetermined number picture elements, are discarded.

48. The method of claim 44, wherein said at least one threshold includes taking into account only picture elements in which a difference in oxygen saturation and/or blood volume is above a predetermined threshold.

49. The method of claim 44, wherein said at least one threshold is effected by discarding clusters of neighboring picture elements which include less than a predetermined number picture elements highlighting differences among spectra of the exposed cortex acquired in steps (b) and (d) of oxygen saturation and/or blood volume differences.

50. The method of claim 6, wherein said step of generating individual spectra-images from spectra acquired in steps (b) and (d) includes generating color or intensity coded saturation and/or blood volume maps.

51. The method of claim 50, wherein said coded saturation maps are coded oxygen saturation maps.

52. The method of claim 50, further comprising the step of generating an anatomical image of the exposed cortex and co-displaying at least one of said color or intensity coded saturation and/or blood volume maps and the anatomical image of the exposed cortex.

53. The method of claim 52, wherein said anatomical image is a monochromatic image.

54. The method of claim 52, wherein said anatomical image is a grayscale image.

55. The method of claim 52, wherein said anatomical image is a red-green-blue image.

56. The method of claim 52, wherein at least one of said color or intensity coded saturation and/or blood volume maps and the anatomical image of the exposed cortex are co-displayed side by side.

57. The method of claim 52, wherein at least one of said color or intensity coded saturation and/or blood volume maps and the anatomical image of the exposed cortex are superimposed.

58. The method of claim 1, wherein said image highlighting differences among spectra of the exposed cortex acquired in steps (b) and (d), so as to highlight functional brain regions, is coded via color or intensity so as to distinguish degree of said differences in accordance with at least one difference threshold.

59. The method of claim 13, wherein said anatomical image is a monochromatic image.

60. The method of claim 13, wherein said anatomical image is a grayscale image.

61. The method of claim 13, wherein said anatomical image is a red-green-blue image.

62. A method of generating an oxygen saturation and/or blood volume difference map of a tissue of a subject, the method comprising the steps of:
(a) illuminating the tissue of the subject with incident light;
(b) at a first time point, acquiring a spectrum of each picture element of the tissue of the subject;
(c) at a second time point, acquiring at least one additional spectrum of each picture element of the tissue of the subject; and
(d) generating an image highlighting differences among spectra of the tissue acquired in steps (b) and (c), so as to generate the oxygen saturation and/or blood volume difference map of the tissue.

63. The method of claim 62, wherein step (d) comprises a use of at least one threshold while generating the image highlighting differences among spectra of the tissue acquired in steps (b) and (c).

64. The method of claim 63, wherein said at least one threshold includes taking into account only picture elements in which, in step (b), in step (d) or both, an absolute oxygen saturation and/or blood is above a predetermined first threshold.

65. The method of claim 64, wherein said at least one threshold further includes taking into account only picture elements in which a difference in oxygen saturation and/or blood is above a predetermined second threshold.

66. The method of claim 65, wherein clusters of neighboring picture elements above said first and said second threshold, said clusters include less than a predetermined number picture elements, are discarded.

67. The method of claim 63, wherein said at least one threshold includes taking into account only picture elements in which a difference in oxygen saturation and/or blood is above a predetermined threshold.

68. The method of claim 62, wherein said at least one threshold is effected by discarding clusters of neighboring picture elements which include less than a predetermined number picture elements highlighting differences among spectra of the tissue acquired in steps (b) and (c).

69. The method of claim 62, further comprising the step of using at least one filter to adjust the spectrum of the incident light.

70. The method of claim 62, wherein each of steps (b) and (c) is independently characterized by spectral resolution ranging between 1 nm and 50 nm and spatial resolution ranging between 0.1 mm and 1.0 mm.

71. The method of claim 62, wherein each of steps (b) and (c) is effected via an interferometer-based spectral imaging device.

72. The method of claim 62, wherein each of steps (b) and (c) is effected via a filters-based spectral imaging device.

73. The method of claim 62, further comprising the steps of generating individual spectra-images from spectra acquired in steps (b) and (c).

74. The method of claim 73, wherein said spectral-images are generated by attributing each of the pixels in the images a distinctive color or intensity according to oxygen saturation and/or blood volume characterizing its respective picture element in the tissue.

75. The method of claim 62, wherein the tissue is selected from the group consisting of a brain, a heart, a liver, a kidney, an eye, a muscle and skin.

76. The method of claim 62, further comprising the step of oxygenating or deoxygenating the tissue between steps (b) and (c).

77. The method of claim 62, further comprising the step of generating an anatomical image of the tissue and co-displaying said oxygen saturation and/or blood volume difference map of the tissue with said anatomical image of the tissue.

78. The method of claim 77, wherein said oxygen saturation and/or blood volume difference map and the anatomical image of the tissue are co-displayed side by side.

79. The method of claim 77, wherein said oxygen saturation and/or blood volume difference map and said anatomical image of the tissue are superimposed.

80. The method of claim 62, wherein step (d) comprises a use of at least one threshold while generating said oxygen saturation and/or blood volume difference map.

81. The method of claim 62, wherein said oxygen saturation and/or blood volume difference map is color or intensity coded.

82. The method of claim 74, wherein said step (d) is characterized by highlighting oxygen saturation and/or blood volume differences of about at least 10%.

83. The method of claim 74, wherein said step (d) is characterized by highlighting oxygen saturation and/or blood volume differences of about at least 5%.

84. The method of claim 72, wherein said filters-based spectral imaging device includes filters selected so as to collect spectral data of intensity peaks or steeps characterizing hemoglobin selected from the group consisting of deoxy-hemoglobin, oxy-hemoglobin and deoxy-hemoglobin and oxy-hemoglobin.

85. The method of claim 84, wherein each of said filters is individually about 5 to about 15 nm full-width-at-half-maximum filter.

86. The method of claim 84, wherein each of said filters is individually about 10 mn full-width-at-half-max filter.

87. The method of claim 84, wherein said filters include N filters selected from the group consisting of an about 540 nm maximal transmittance filter, an about 575 nm maximal transmittance filter, an about 555 nm maximal transmittance filter, an about 513 nm maximal transmittance filter and an about 600 nm maximal transmittance filter, whereas N is an integer selected from the group consisting two, three, four and five.

88. The method of claim 87, wherein N equals two.

89. The method of claim 87, wherein N equals three.

90. The method of claim 87, wherein N equals four.

91. The method of claim 87, wherein N equals five.

92. The method of claim 84, wherein said filters include at least one multiple chroic filter.

93. The method of claim 84, wherein said filters include at least one filter of maximal transmittance at a wavelength which corresponds to at least one isosbasthic point of deoxy-hemoglobin and oxy-hemoglobin and at least one additional filter of maximal transmittance at a wavelength which corresponds to at least one non-isosbasthic point of deoxy-hemoglobin and oxy-hemoglobin.

94. The method of claim 62, wherein said reflectance spectrum of step (b) is an averaged reference spectrum of N measurements, wherein N is an integer and equals at least 2.

95. The method of claim 62, wherein said reflectance spectrum of step (c) is an averaged reference spectrum, wherein N is an integer and equals at least 2.

96. The method of claim 62, further comprising the steps of spatially registrating spectral data acquired in steps (b) and (c).

97. The method of claim 73, wherein said step of generating individual spectra-images from spectra acquired in steps (b) and (c) includes generating color or intensity coded oxygen saturation and/or blood volume maps.

98. The method of claim 97, further comprising the step of generating an anatomical image of the tissue and co-displaying at least one of said color or intensity coded oxygen saturation and/or blood volume maps and the anatomical image of the tissue.

99. The method of claim 98, wherein said anatomical image is a monochromatic image.

100. The method of claim 98, wherein said anatomical image is a grayscale image.

101. The method of claim 98, wherein said anatomical image is a red-green-blue image.

102. The method of claim 98, wherein at least one of said color or intensity coded oxygen saturation and/or blood volume maps and the anatomical image of the tissue are co-displayed side by side.

103. The method of claim 98, wherein at least one of said color or intensity coded oxygen saturation and/or blood volume maps and the anatomical image of the tissue are superimposed.

104. The method of claim 62, wherein said oxygen saturation and/or blood volume difference map is coded via color or intensity so as to distinguish degree of said differences in accordance with at least one difference threshold.

105. The method of claim 77, wherein said anatomical image is a monochromatic image.

106. The method of claim 77, wherein said anatomical image is a grayscale image.

107. The method of claim 77, wherein said anatomical image is a red-green-blue image.

108. A method of performing a neurosurgery for the removal of a mass from a brain of a subject while minimizing damage to a neighboring brain tissue, the method comprising the steps of:
(a) performing a craniotomy so as to expose at least a portion of a cortex of the subject;
(b) performing functional brain mapping of the subject by:
(i) illuminating the exposed portion of the cortex with incident light;
(ii) acquiring a reflectance spectrum of each picture element of at least a portion of the exposed cortex of the subject;
(iii) stimulating the neighboring brain tissue of the subject;
(iv) during or after step (iii) acquiring at least one additional reflectance spectrum of each picture element of at least the portion of the exposed cortex of the subject; and
(v) generating an image highlighting differences among spectra of the exposed cortex acquired in steps (ii) and (iv), so as to highlight the functional brain regions of the neighboring brain tissue; and
(c) assisted by said image, removing the mass while minimizing damage to the neighboring brain tissue.

109. The method of claim 108, further comprising the step of using at least one filter to adjust the spectrum of the incident light.

110. The method of claim 108, wherein each of steps (ii) and (iv) is independently characterized by spectral resolution ranging between 1 nm and 50 nm and spatial resolution ranging between 0.1 mm and 1.0 mm.

111. The method of claim 108, wherein each of steps (ii) and (iv) is effected via an interferometer-based spectral imaging device.

112. The method of claim 108, wherein each of steps (ii) and (iv) is effected via a filters-based spectral imaging device.

113. The method of claim 108, further comprising the steps of generating individual spectra-images from spectra acquired in steps (ii) and (iv).

114. The method of claim 113, wherein said spectral-images are generated by attributing each of the pixels in the images a distinctive color or intensity according to oxygen saturation and/or blood volume characterizing its respective picture element in the cortex.

115. The method of claim 108, wherein the subject is awake.

116. The method of claim 108, wherein the subject is anesthetized.

117. The method of claim 108, wherein step (c) is effected by asking the subject to perform a task.

118. The method of claim 117, wherein said task is selected from the group consisting of reading, speaking, listening, viewing, memorizing, thinking and executing a voluntary action.

119. The method of claim 108, wherein step (c) is effected by a method selected from the group consisting of passively stimulating the brain through the peripheral nervous system of the subject and directly stimulating the cortex.

120. The method of claim 108, further comprising the step of generating an anatomical image of the exposed cortex and co-displaying said image highlighting differences among spectra of the exposed cortex and the anatomical image of the exposed cortex.

121. The method of claim 120, wherein said image highlighting differences among spectra of the exposed cortex and the anatomical image of the exposed cortex are co-displayed side by side.

122. The method of claim 120, wherein said image highlighting differences among spectra of the exposed cortex and the anatomical image of the exposed cortex are superimposed.

123. The method of claim 108, wherein step (e) comprises a use of at least one threshold while generating the image highlighting differences among spectra of the exposed cortex acquired in steps (ii) and (iv).

124. The method of claim 108, wherein said image highlighting differences among spectra of the exposed cortex acquired in steps (ii) and (iv) is color or intensity coded.

125. The method of claim 108, wherein medical lines are connected to the subject on a single side thereof.

126. The method of claim 108, wherein medical lines are connected to the subject on a single side thereof.

127. The method of claim 108, wherein medical lines are connected to the subject at locations which are less communicating with the exposed portion of the cortex of the subject.

128. The method of claim 114, wherein said step (v) is characterized by highlighting oxygen saturation and/or blood volume differences of about at least 10%.

129. The method of claim 114, wherein said step (v) is characterized by highlighting oxygen saturation and/or blood volume differences of about at least 5%.

130. The method of claim 115, further comprising the step of also acquiring a reflectance spectrum of each picture element of at least the portion of the exposed cortex of the subject when the patient is briefly anesthetized.

131. The method of claim 108, wherein each of steps (ii) and (iv) is performed during at least N brain beats of the subject, wherein N is an integer selected from the group consisting of two, three, four, five, six, seven, eight, nine, ten and an integer between and including eleven and forty.

132. The method of claim 108, wherein step (d) is executed more than about 3-5 seconds after initiation of step (c).

133. The method of claim 108, wherein step (d) is executed between about 5 and about 30 seconds after initiation of step (c).

134. The method of claim 108, wherein said stimulation prolongs about 5 to about 30 seconds.

135. The method of claim 108, wherein said stimulation prolongs about 10 to about 20 seconds.

136. The method of claim 112, wherein said filters-based spectral imaging device includes filters selected so as to collect spectral data of intensity peaks or steeps characterizing one or more spectrally monitored substances.

137. The method of claim 112, wherein said filters-based spectral imaging device includes filters selected so as to collect spectral data of intensity peaks or steeps characterizing hemoglobin selected from the group consisting of deoxy-hemoglobin, oxy-hemoglobin and deoxy-hemoglobin and oxy-hemoglobin.

138. The method of claim 137, wherein each of said filters is individually about 5 to about 15 nm full-width-at-half-maximum filter.

139. The method of claim 137, wherein each of said filters is individually about 10 nm full-width-at-half-max filter.

140. The method of claim 137, wherein said filters include N filters selected from the group consisting of an about 540 nm maximal transmittance filter, an about 575 nm maximal transmittance filter, an about 555 nm maximal transmittance filter, an about 513 nm maximal transmittance filter and an about 600 nm maximal transmittance filter, whereas N is an integer selected from the group consisting two, three, four and five.

141. The method of claim 140, wherein N equals two.

142. The method of claim 140, wherein N equals three.

143. The method of claim 140, wherein N equals four.

144. The method of claim 140, wherein N equals five.

145. The method of claim 137, wherein said filters include at least one multiple chroic filter.

146. The method of claim 137, wherein said filters include at least one filter of maximal transmittance at a wavelength which corresponds to at least one isosbasthic point of deoxy-hemoglobin and oxy-hemoglobin and at least one additional filter of maximal transmittance at a wavelength which corresponds to at least one non-isosbasthic point of deoxy-hemoglobin and oxy-hemoglobin.

147. The method of claim 108, wherein said reflectance spectrum of step (b) is an averaged reference spectrum of N measurements, wherein N is an integer and equals at least 2.

148. The method of claim 108, wherein said reflectance spectrum of step (d) is an averaged reference spectrum, wherein N is an integer and equals at least 2.

149. The method of claim 108, further comprising the steps of spatially registrating spectral data acquired in steps (ii) and (iv).

150. The method of claim 108, wherein said image highlighting differences among spectra of the exposed cortex acquired in steps (ii) and (iv) is highlighting oxygen saturation and/or blood volume differences.

151. The method of claim 150, wherein step (e) comprises a use of at least one threshold while generating the image highlighting differences among spectra of the exposed cortex acquired in steps (ii) and (iv) of oxygen saturation and/or blood volume differences.

152. The method of claim 151, wherein said at least one threshold includes taking into account only picture elements in which, in step (b), in step (d) or both an absolute oxygen saturation and/or blood volume is above a predetermined first threshold.

153. The method of claim 152, wherein said at least one threshold further includes taking into account only picture elements in which a difference in oxygen saturation and/or blood volume is above a predetermined second threshold.

154. The method of claim 153, wherein clusters of neighboring picture elements above said first and said second threshold, said clusters include less than a predetermined number picture elements, are discarded.

155. The method of claim 151, wherein said at least one threshold includes taking into account only picture elements in which a difference in oxygen saturation and/or blood volume is above a predetermined threshold.

156. The method of claim 151, wherein said at least one threshold is effected by discarding clusters of neighboring picture elements which include less than a predetermined number picture elements highlighting differences among spectra of the exposed cortex acquired in steps (ii) and (iv) of oxygen saturation and/or blood volume differences.

157. The method of claim 113, wherein said step of generating individual spectra-images from spectra acquired in steps (ii) and (iv) includes generating color or intensity coded saturation and/or blood volume maps.

158. The method of claim 157, wherein said coded saturation maps are coded oxygen saturation maps.

159. The method of claim 157, further comprising the step of generating an anatomical image of the exposed cortex and co-displaying at least one of said color or intensity coded saturation and/or blood volume maps and the anatomical image of the exposed cortex.

160. The method of claim 159, wherein said anatomical image is a monochromatic image.

161. The method of claim 159, wherein said anatomical image is a grayscale image.

162. The method of claim 159, wherein said anatomical image is a red-green-blue image.

163. The method of claim 159, wherein at least one of said color or intensity coded saturation and/or blood volume maps and the anatomical image of the exposed cortex are co-displayed side by side.

164. The method of claim 159, wherein at least one of said color or intensity coded saturation and/or blood volume maps and the anatomical image of the exposed cortex are superimposed.

165. The method of claim 108, wherein said image highlighting differences among spectra of the exposed cortex acquired in steps (ii) and (iv), so as to highlight functional brain regions, is coded via color or intensity so as to distinguish degree of said differences in accordance with at least one difference threshold.

166. The method of claim 120, wherein said anatomical image is a monochromatic image.

167. The method of claim 120, wherein said anatomical image is a grayscale image.

168. The method of claim 120, wherein said anatomical image is a red-green-blue image.

169. A system for functional brain mapping of a subject, the system comprising:
(a) an illumination device for illuminating an exposed cortex of a brain or portion thereof of the subject with incident light;
(b) a spectral imaging device for acquiring reflectance spectra of each picture element of at least a portion of the exposed cortex of the subject before and during and/or after stimulating the brain of the subject; and
(c) an image generating device for generating an image highlighting differences among spectra of the exposed cortex acquired before and during and/or after stimulating the brain of the subject, so as to highlight functional brain regions.

170. The system of claim 169, further comprising at least one filter being engaged with said illumination device to adjust the spectrum of the incident light.

171. The system of claim 169, so designed and constructed so as to provide spectral resolution ranging between 1 nm and 50 nm and spatial resolution ranging between 0.1 mm and 1.0 mm.

172. The system of claim 169, wherein said spectral imaging device is an interferometer-based spectral imaging device.

173. The system of claim 169, wherein said spectral imaging device is a filters-based spectral imaging device.

174. The system of claim 169, wherein said image generating device is designed and constructed for generating individual spectra-images from spectra of the exposed cortex acquired before and during and/or after stimulating the brain of the subject.

175. The system of claim 174, wherein said spectral-images are generated by attributing each of the pixels in the images a distinctive color or intensity according to oxygen saturation and/or blood volume and/or blood volume characterizing its respective picture element in the cortex.

176. The system of claim 169, wherein said image generating device is designed and constructed for generating an anatomical image of the exposed cortex and co-displaying said image highlighting differences among spectra of the exposed cortex and the anatomical image of the exposed cortex.

177. The system of claim 176, wherein said image highlighting differences among spectra of the exposed cortex and the anatomical image of the exposed cortex are co-displayed by said image generating device side by side.

178. The system of claim 176, wherein said image highlighting differences among spectra of the exposed cortex and the anatomical image of the exposed cortex are superimposed by said image generating device.

179. The system of claim 169, wherein said image generating device uses at least one threshold while generating the image highlighting differences among spectra of the exposed cortex.

180. The system of claim 169, wherein said image highlighting differences among spectra of the exposed cortex is color or intensity coded by said image generating device.

181. The system of claim 175, wherein said image generating device is set to highlight oxygen saturation and/or blood volume differences of about at least 10%.

182. The system of claim 175, wherein said image generating device is set to highlight oxygen saturation and/or blood volume differences of about at least 5%.

183. The system of claim 169, wherein said spectral imaging device is set for acquiring said reflectance spectra of each of said picture element of at least said portion of the exposed cortex of the subject before and during and/or after stimulating the brain of the subject during at least N brain beats of the subject, wherein N is an integer selected from the group consisting of two, three, four, five, six, seven, eight, nine, ten and an integer between and including eleven and forty.

184. The system of claim 173, wherein said filters-based spectral imaging device includes filters selected so as to collect spectral data of intensity peaks or steeps characterizing one or more spectrally monitored substances.

185. The system of claim 173, wherein said filters-based spectral imaging device includes filters selected so as to collect spectral data of intensity peaks or steeps characterizing hemoglobin selected from the group consisting of deoxy-hemoglobin, oxy-hemoglobin and deoxy-hemoglobin and oxy-hemoglobin.

186. The system of claim 185, wherein each of said filters is individually about 5 to about 15 nm full-width-at-half-maximum filter.

187. The system of claim 185, wherein each of said filters is individually about 10 nm full-width-at-half-max filter.

188. The system of claim 185, wherein said filters include N filters selected from the group consisting of an about 540 nm maximal transmittance filter, an about 575 nm maximal transmittance filter, an about 555 nm maximal transmittance filter, an about 513 mn maximal transmittance filter and an about 600 nm maximal transmittance filter, whereas N is an integer selected from the group consisting two, three, four and five.

189. The system of claim 188, wherein N equals two.

190. The system of claim 188, wherein N equals three.

191. The system of claim 188, wherein N equals four.

192. The system of claim 188, wherein N equals five.

193. The system of claim 185, wherein said filters include at least one multiple chroic filter.

194. The system of claim 185, wherein said filters include at least one filter of maximal transmittance at a wavelength which corresponds to at least one isosbasthic point of deoxy-hemoglobin and oxy-hemoglobin and at least one additional filter of maximal transmittance at a wavelength which corresponds to at least one non-isosbasthic point of deoxy-hemoglobin and oxy-hemoglobin.

195. The system of claim 169, wherein said spectral imaging device is designed and constructed for spatially registrating spectral data acquired thereby.

196. The system of claim 169, wherein said image generating device is designed and constructed for highlighting differences among oxygen saturation and/or blood volume of the cortex.

197. The system of claim 196, wherein said image generating device is designed for use of at least one threshold while generating the image highlighting differences among said oxygen saturation and/or blood volume of the cortex.

198. The system of claim 197, wherein said at least one threshold includes taking into account only picture elements in which, before, during and/or after said stimulation, an, absolute oxygen saturation and/or blood volume is above a predetermined first threshold.

199. The system of claim 198, wherein said at least one threshold further includes taking into account only picture elements in which a difference in oxygen saturation and/or blood volume is above a predetermined second threshold.

200. The system of claim 199, wherein clusters of neighboring picture elements above said first and said second threshold, said clusters include less than a predetermined number picture elements, are discarded.

201. The system of claim 197, wherein said at least one threshold includes taking into account only picture elements in which a difference in oxygen saturation and/or blood volume is above a predetermined threshold.

202. The system of claim 197, wherein said at least one threshold is effected by discarding clusters of neighboring picture elements which include less than a predetermined number picture elements highlighting differences among oxygen saturation and/or blood volume of the cortex.

203. The system of claim 174, wherein said individual spectra-images are color or intensity coded saturation and/or blood volume maps.

204. The system of claim 203, wherein said coded saturation and/or blood volume maps are coded oxygen saturation and/or blood volume maps.

205. The system of claim 203, wherein said image generating device is designed and constructed for generating an anatomical image of the exposed cortex and co-displaying at least one of said color or intensity coded saturation and/or blood volume maps and the anatomical image of the exposed cortex.

206. The system of claim 205, wherein said anatomical image is a monochromatic image.

207. The system of claim 205, wherein said anatomical image is a grayscale image.

208. The system of claim 205, wherein said anatomical image is a red-green-blue image.

209. The system of claim 205, wherein at least one of said color or intensity coded saturation and/or blood volume maps and the anatomical image of the exposed cortex are co-displayed side by side.

210. The system of claim 205, wherein at least one of said color or intensity coded saturation and/or blood volume maps and the anatomical image of the exposed cortex are superimposed.

211. The system of claim 169, wherein said image generating device is designed and constructed to distinguish degree of said differences in accordance with at. least one difference threshold.

212. The system of claim 176, wherein said anatomical image is a monochromatic image.

213. The system of claim 176, wherein said anatomical image is a grayscale image.

214. The system of claim 176, wherein said anatomical image is a red-green-blue image.

215. A system of generating an oxygen saturation and/or blood volume difference map of a tissue of a subject, the system comprising:
(a) an illumination device for illuminating the tissue of the subject with incident light;
(b) a spectral imaging device for acquiring spectra of each picture element of the tissue of the subject at a first time point and at a second time point; and
(c) an image generating device for generating an image highlighting differences among spectra of the tissue acquired in said first and said second time points, so as to generate the oxygen saturation and/or blood volume difference map of the tissue.

216. The system of claim 215, wherein said image generating device uses of at least one threshold while generating the image highlighting differences among spectra of the tissue acquired in said first and said second time points.

217. The system of claim 216, wherein said at least one threshold includes taking into account only picture elements in which, in said first time point, in said second time point, or both, an absolute oxygen saturation and/or blood volume is above a predetermined first threshold.

218. The system of claim 217, wherein said at least one threshold further includes taking into account only picture elements in which a difference in oxygen saturation and/or blood volume is above a predetermined second threshold.

219. The system of claim 218, wherein clusters of neighboring picture elements above said first and said second threshold, said clusters include less than a predetermined number picture elements, are discarded.

220. The system of claim 216, wherein said at least one threshold includes taking into account only picture elements in which a difference in oxygen saturation and/or blood volume is above a predetermined threshold.

221. The system of claim 215, wherein said at least one threshold is effected by discarding clusters of neighboring picture elements which include less than a predetermined number picture elements highlighting differences among spectra of the tissue acquired in said first and said second time points.

222. The system of claim 215, further comprising at least one filter engaging said illumination device to adjust the spectrum of the incident light.

223. The system of claim 215, wherein spectral data acquired at each of said first and said second time points is independently characterized by spectral resolution ranging between 1 nm and 50 nm and spatial resolution ranging between 0.1 mm and 1.0 mm.

224. The system of claim 215, wherein spectral data acquired at each of said first and said second time points is collected via an interferometer-based spectral imaging device.

225. The system of claim 215, wherein spectral data acquired at each of said first and said second time points is collected via a filters-based spectral imaging device.

226. The system of claim 215, wherein said image generating device is designed and constructed for generating individual spectral-images from spectra acquired during said first and said second time points.

227. The system of claim 226, wherein said spectral-images are generated by attributing each of the pixels in the images a distinctive color or intensity according to oxygen saturation and/or blood volume characterizing its respective picture element in the cortex.

228. The system of claim 215, wherein said image generating device is designed and constructed for generating an anatomical image of the tissue and co-displaying said oxygen saturation and/or blood volume difference map of the tissue with said anatomical image of the tissue.

229. The system of claim 228, wherein said oxygen saturation and/or blood volume difference map and the anatomical image of the tissue are co-displayed side by side by said image generating device.

230. The system of claim 228, wherein said oxygen saturation and/or blood volume difference map and said anatomical image of the tissue are superimposed image generating device.

231. The system of claim 215, wherein said oxygen saturation and/or blood volume difference map is color or intensity coded.

232. The system of claim 225, wherein said filters-based spectral imaging device includes filters selected so as to collect spectral data of intensity peaks or steeps characterizing hemoglobin selected from the group consisting of deoxy-hemoglobin, oxy-hemoglobin and deoxy-hemoglobin and oxy-hemoglobin.

233. The system of claim 232, wherein each of said filters is individually about 5 to about 15 nm fill-width-at-half-maximum filter.

234. The system of claim 232, wherein each of said filters is individually about 10 nm fill-width-at-half-max filter.

235. The system of claim 232, wherein said filters include N filters selected from the group consisting of an about 540 nm maximal transmittance filter, an about 575 nm maximal transmittance filter, an about 555 nm maximal transmittance filter, an about 513 nm maximal transmittance filter and an about 600 nm maximal transmittance filter, whereas N is an integer selected from the group consisting two, three, four and five.

236. The system of claim 235, wherein N equals two.

237. The system of claim 235, wherein N equals three.

238. The system of claim 235, wherein N equals four.

239. The system of claim 235, wherein N equals five.

240. The system of claim 232, wherein said filters include at least on e multiple chroic filter.

241. The system of claim 232, wherein said filters include at least one filter of maximal transmittance at a wavelength which corresponds to at least one isosbasthic point of deoxy-hemoglobin and oxy-hemoglobin and at least one additional filter of maximal transmittance at a wavelength which corresponds to at least one non-isosbasthic point of deoxy-hemoglobin and oxy-hemoglobin.

242. The system of claim 226, wherein said individual spectra-images are color or intensity coded oxygen saturation and/or blood volume maps.

243. A system for monitoring oxygen saturation in a tissue comprising a spectral imaging device and an image generating device, said spectral imaging device and said image generating device acting in synergy to produce an oxygen saturation difference map by highlighting tissue regions characterized by a characteristic selected from the group consisting of:
(a) having an absolute or relative level of oxygen saturation above a predetermined first threshold;
(b) having an oxygen saturation difference above a predetermined second threshold; and
(c) having a cluster size above a predetermined size.

244. A system for monitoring oxygen saturation in a tissue comprising a spectral imaging device and an image generating device, said spectral imaging device and said image generating device acting in synergy to produce an oxygen saturation difference map by highlighting tissue regions characterized by:
(a) having an absolute or relative level of oxygen saturation above a predetermined first threshold;
(b) having an oxygen saturation difference above a predetermined second threshold; and
(c) having a cluster size above a predetermined size.

245. A system for monitoring blood volume in a tissue comprising a spectral imaging device and an image generating device, said spectral imaging device and said image generating device acting in synergy to produce a blood volume difference map by highlighting tissue regions characterized by a characteristic selected from the group consisting of:
(a) having an absolute or relative level of blood volume above a predetermined first threshold;
(b) having a blood volume difference above a predetermined second threshold; and
(c) having a cluster size above a predetermined size.

246. A system for monitoring blood volume in a tissue comprising a spectral imaging device and an image generating device, said spectral imaging device and said image generating device acting in synergy to produce a blood volume difference map by highlighting tissue regions characterized by:
(a) having an absolute or relative level of blood volume above a predetermined first threshold;
(b) having a blood volume difference above a predetermined second threshold; and
(c) having a cluster size above a predetermined size.

247. A system for functional brain mapping comprising a spectral imaging device and an image generating device, said spectral imaging device and said image generating device acting in synergy to produce an anatomical image of the brain or a portion thereof and a coded functional map of the brain or said portion thereof, said coded functional map reflecting a change in the brain in response to a stimulus, said functional map and said anatomical image being co-displayed.

248. The method of claim 62, wherein said reflectance spectrum of step (b) is an averaged reference spectrum of N brain beats, wherein N is an integer and equals at least 2.

249. The method of claim 1, wherein said reflectance spectrum of step (b) is an averaged reference spectrum of N brain beats, wherein N is an integer and equals at least 2.

250. The method of claim 108, wherein said reflectance spectrum of step (b) is an averaged reference spectrum of N brain beats, wherein N is an integer and equals at least 2.

251. A method of brain mapping of a subject comprising the steps of:
(a) illuminating an exposed cortex of a brain or portion thereof of the subject with incident light;
(b) acquiring a reflectance spectrum of each picture element of at least a portion of the exposed cortex of the subject; and
(e) generating an image highlighting concentrations of at least one substance in the brain.

252. The system of claim 251, wherein a plurality of images highlighting differences among spectra are displayed either superimposed, overlaid or integrated.

253. The method of claim 1, wherein a plurality of images highlighting differences among spectra are displayed either superimposed, overlaid or integrated.

254. The method of claim 5, wherein step (a) is effected by an illumination device operated with an alternating current characterized by a frequency time.

255. The method of claim 254, wherein (i) an exposure time of all filters of said filters-based spectral imaging device is substantially equal; and (ii) an exposure time of each of said filters is a multiplicity of said frequency time by an integer.

256. The method of claim 62, wherein a plurality of images highlighting differences among spectra are displayed either superimposed, overlaid or integrated.

257. The method of claim 72, wherein step (a) is effected by an illumination device operated with an alternating current characterized by a frequency time.

258. The method of claim 257, wherein (i) an exposure time of all filters of said filters-based spectral imaging device is substantially equal; and (ii) an exposure time of each of said filters is a multiplicity of said frequency time by an integer.

259. The method of claim 108, wherein a plurality of images highlighting differences among spectra are displayed either superimposed, overlaid or integrated.

260. The method of claim 112, wherein step (b)(i) is effected by an illumination device operated with an alternating current characterized by a frequency time.

261. The method of claim 260, wherein (i) an exposure time of all filters of said filters-based spectral imaging device is substantially equal; and (ii) an exposure time of each of said filters is a multiplicity of said frequency time by an integer.

262. The system of claim 169, wherein a plurality of images highlighting differences among spectra are displayed either superimposed, overlaid or integrated.

263. The system of claim 173, wherein said illumination device is operated with an alternating current characterized by a frequency time.

264. The system of claim 263, wherein (i) an exposure time of all filters of said filters-based spectral imaging device is substantially equal; and (ii) an exposure time of each of said filters is a multiplicity of said frequency time by an integer.

265. The system of claim 215, wherein a plurality of images highlighting differences among spectra are displayed either superimposed, overlaid or integrated.

266. The system of claim 225, wherein said illumination device operated with an alternating current characterized by a frequency time.

267. The system of claim 266, wherein (i) an exposure time of all filters of said filters-based spectral imaging device is substantially equal; and (ii) an exposure time of each of said filters is a multiplicity of said frequency time by an integer.

268. The method of claim 13, wherein the anatomical image includes text identifying brain portions.

269. The method of claim 1, wherein at least one orientation element is placed on the exposed portion of the cortex prior to step (b), so as to provide orientation.

270. The method of claim 269, wherein said at least one orientation element also serves as a white target.

271. The method of claim 270, wherein at least a portion of said at least one orientation element has an index of refraction close to an index of refraction of the cortex.
